SEQUENCE LISTING (924 sequences)

SEQ ID NO: 1 - DNA, 33 bp, Homo sapiens
cattttgtttcattaacccaagacttaatttgg

SEQ ID NO: 2 - DNA, 33 bp, Homo sapiens
cattttgtttcattaatccaagacttaatttgg

SEQ ID NO: 3 - DNA, 33 bp, Homo sapiens
atagggagaatgctcactaatggtgaacatctc

SEQ ID NO: 4 - DNA, 33 bp, Homo sapiens
atagggagaatgctcattaatggtgaacatctc

SEQ ID NO: 5 - DNA, 33 bp, Homo sapiens
tatcatagcacttatacagtgttgtgccattgt

SEQ ID NO: 6 - DNA, 33 bp, Homo sapiens
tatcatagcacttatagagtgttgtgccattgt

SEQ ID NO: 7 - DNA, 33 bp, Homo sapiens
ttaagtcacttgcccaagattattcaggacgta

SEQ ID NO: 8 - DNA, 33 bp, Homo sapiens
ttaagtcacttgcccaggattattcaggacgta

SEQ ID NO: 9 - DNA, 33 bp, Homo sapiens
tgtcagatctgcggaaagatgagtagaatgggt

SEQ ID NO: 10 - DNA, 33 bp, Homo sapiens
tgtcagatctgcggaacgatgagtagaatgggt

SEQ ID NO: 11 - DNA, 33 bp, Homo sapiens
gctaagggccttcaaaatttctattgctttaca

SEQ ID NO: 12 - DNA, 33 bp, Homo sapiens
gctaagggccttcaaagtttctattgctttaca

SEQ ID NO: 13 - DNA, 33 bp, Homo sapiens
aaatgaaaacacacaaattgaaaatgcacagaa

SEQ ID NO: 14 - DNA, 33 bp, Homo sapiens
aaatgaaaacacacaagttgaaaatgcacagaa

SEQ ID NO: 15 - DNA, 33 bp, Homo sapiens
cttgaaatttcctctaggatagcttctctttgg

SEQ ID NO: 16 - DNA, 33 bp, Homo sapiens
cttgaaatttcctctatgatagcttctctttgg

SEQ ID NO: 17 - DNA, 33 bp, Homo sapiens
cccctggcatagtagtcaaaggctgtaggagcc

SEQ ID NO: 18 - DNA, 33 bp, Homo sapiens
cccctggcatagtagttaaaggctgtaggagcc

SEQ ID NO: 19 - DNA, 33 bp, Homo sapiens
gttgtcttagaagccacgggaagcaaacgtatg

SEQ ID NO: 20 - DNA, 33 bp, Homo sapiens
gttgtcttagaagccatgggaagcaaacgtatg

SEQ ID NO: 21 - DNA, 33 bp, Homo sapiens
gattttctgccaagacaataagaagagtacttt

SEQ ID NO: 22 - DNA, 33 bp, Homo sapiens
gattttctgccaagacgataagaagagtacttt

SEQ ID NO: 23 - DNA, 33 bp, Homo sapiens
ctagtgcatcattgaacgaaaggaatttcacac

SEQ ID NO: 24 - DNA, 33 bp, Homo sapiens
ctagtgcatcattgaaggaaaggaatttcacac

SEQ ID NO: 25 - DNA, 33 bp, Homo sapiens
taagagaatgaatgtccagccaaccactggatt

SEQ ID NO: 26 - DNA, 33 bp, Homo sapiens
taagagaatgaatgtctagccaaccactggatt

SEQ ID NO: 27 - DNA, 33 bp, Homo sapiens
gacacatttgctaaaactatacaatcacagcaa

SEQ ID NO: 28 - DNA, 33 bp, Homo sapiens
gacacatttgctaaaattatacaatcacagcaa

SEQ ID NO: 29 - DNA, 33 bp, Homo sapiens
aatatttttctctacccgaatccatcagctacc

SEQ ID NO: 30 - DNA, 33 bp, Homo sapiens
aatatttttctctaccggaatccatcagctacc

SEQ ID NO: 31 - DNA, 33 bp, Homo sapiens
ggtgccaagctagtcaatcagtggcaaagctca

SEQ ID NO: 32 - DNA, 33 bp, Homo sapiens
ggtgccaagctagtcagtcagtggcaaagctca

SEQ ID NO: 33 - DNA, 33 bp, Homo sapiens
ggtttgtgtgtacttagctaatttttaaagaag

SEQ ID NO: 34 - DNA, 33 bp, Homo sapiens
ggtttgtgtgtacttatctaatttttaaagaag

SEQ ID NO: 35 - DNA, 33 bp, Homo sapiens
aggtggaccttcagacaatcttaaagtgtcagt

SEQ ID NO: 36 - DNA, 33 bp, Homo sapiens
aggtggaccttcagacgatcttaaagtgtcagt

SEQ ID NO: 37 - DNA, 33 bp, Homo sapiens
agtgagacatggatgacacccacacttcactct

SEQ ID NO: 38 - DNA, 33 bp, Homo sapiens
agtgagacatggatgatacccacacttcactct

SEQ ID NO: 39 - DNA, 33 bp, Homo sapiens
caggcaaatgtccaaacgagcattaggtgtacg

SEQ ID NO: 40 - DNA, 33 bp, Homo sapiens
caggcaaatgtccaaatgagcattaggtgtacg

SEQ ID NO: 41 - DNA, 33 bp, Homo sapiens
taaatcaagtgtttaaagagggacattcacgtc

SEQ ID NO: 42 - DNA, 33 bp, Homo sapiens
taaatcaagtgtttaaggagggacattcacgtc

SEQ ID NO: 43 - DNA, 33 bp, Homo sapiens
gggctagggtttcctgcagtatgcaataacatt

SEQ ID NO: 44 - DNA, 33 bp, Homo sapiens
gggctagggtttcctgtagtatgcaataacatt

SEQ ID NO: 45 - DNA, 33 bp, Homo sapiens
cctgaaagccataaaacaactgctaagtttttc

SEQ ID NO: 46 - DNA, 33 bp, Homo sapiens
cctgaaagccataaaataactgctaagtttttc

SEQ ID NO: 47 - DNA, 33 bp, Homo sapiens
ttaaatggccctcaaaagatactcctcttacaa

SEQ ID NO: 48 - DNA, 33 bp, Homo sapiens
ttaaatggccctcaaaggatactcctcttacaa

SEQ ID NO: 49 - DNA, 33 bp, Homo sapiens
ctttagaaaacagagacggtgtgcttccagcac

SEQ ID NO: 50 - DNA, 33 bp, Homo sapiens
ctttagaaaacagagatggtgtgcttccagcac

SEQ ID NO: 51 - DNA, 33 bp, Homo sapiens
agggccgaaagagtagacgagggctggtggtct

SEQ ID NO: 52 - DNA, 33 bp, Homo sapiens
agggccgaaagagtagccgagggctggtggtct

SEQ ID NO: 53 - DNA, 33 bp, Homo sapiens
tgaaatttattctgcacgaaatgggcaaaccaa

SEQ ID NO: 54 - DNA, 33 bp, Homo sapiens
tgaaatttattctgcaggaaatgggcaaaccaa

SEQ ID NO: 55 - DNA, 33 bp, Homo sapiens
agaaatgagttgcccacctaactagggtgatgt

SEQ ID NO: 56 - DNA, 33 bp, Homo sapiens
agaaatgagttgcccatctaactagggtgatgt

SEQ ID NO: 57 - DNA, 33 bp, Homo sapiens
ataggtttgtgttttacccttgatcaattttga

SEQ ID NO: 58 - DNA, 33 bp, Homo sapiens
ataggtttgtgttttatccttgatcaattttga

SEQ ID NO: 59 - DNA, 33 bp, Homo sapiens
ttgccgtccacagcaggctactgacgaaaaacg

SEQ ID NO: 60 - DNA, 33 bp, Homo sapiens
ttgccgtccacagcagtctactgacgaaaaacg

SEQ ID NO: 61 - DNA, 33 bp, Homo sapiens
gaaacattaccatattcaagacagtgaacaagt

SEQ ID NO: 62 - DNA, 33 bp, Homo sapiens
gaaacattaccatattgaagacagtgaacaagt

SEQ ID NO: 63 - DNA, 33 bp, Homo sapiens
aaggaagagtttgacacgaagtaaaagtctctg

SEQ ID NO: 64 - DNA, 33 bp, Homo sapiens
aaggaagagtttgacatgaagtaaaagtctctg

SEQ ID NO: 65 - DNA, 33 bp, Homo sapiens
atatgttattctctccaggaattgaatttactc

SEQ ID NO: 66 - DNA, 33 bp, Homo sapiens
atatgttattctctcccggaattgaatttactc

SEQ ID NO: 67 - DNA, 33 bp, Homo sapiens
ctttgtgcgagcgcagacatccccattccatgc

SEQ ID NO: 68 - DNA, 33 bp, Homo sapiens
ctttgtgcgagcgcaggcatccccattccatgc

SEQ ID NO: 69 - DNA, 33 bp, Homo sapiens
tacagatcaagatgtcaacaaggttggttttct

SEQ ID NO: 70 - DNA, 33 bp, Homo sapiens
tacagatcaagatgtcgacaaggttggttttct

SEQ ID NO: 71 - DNA, 33 bp, Homo sapiens
aagagatgtatttcaaaatgcgaaattaacagt

SEQ ID NO: 72 - DNA, 33 bp, Homo sapiens
aagagatgtatttcaagatgcgaaattaacagt

SEQ ID NO: 73 - DNA, 33 bp, Homo sapiens
cagagcagacagctccagagaagaggaactcaa

SEQ ID NO: 74 - DNA, 33 bp, Homo sapiens
cagagcagacagctccggagaagaggaactcaa

SEQ ID NO: 75 - DNA, 33 bp, Homo sapiens
tgccagagtcatgtgaacttgtggagtagatgc

SEQ ID NO: 76 - DNA, 33 bp, Homo sapiens
tgccagagtcatgtgaccttgtggagtagatgc

SEQ ID NO: 77 - DNA, 33 bp, Homo sapiens
ctgtgctcagcttcagaatcagcccagggagat

SEQ ID NO: 78 - DNA, 33 bp, Homo sapiens
ctgtgctcagcttcagtatcagcccagggagat

SEQ ID NO: 79 - DNA, 33 bp, Homo sapiens
aattcataactataataagcatcaagttaacta

SEQ ID NO: 80 - DNA, 33 bp, Homo sapiens
aattcataactataatgagcatcaagttaacta

SEQ ID NO: 81 - DNA, 33 bp, Homo sapiens
caacttacagatatacaatagacatgaataacc

SEQ ID NO: 82 - DNA, 33 bp, Homo sapiens
caacttacagatatacgatagacatgaataacc

SEQ ID NO: 83 - DNA, 33 bp, Homo sapiens
cttagtttctggatagcccatgatatagcagtc

SEQ ID NO: 84 - DNA, 33 bp, Homo sapiens
cttagtttctggatagtccatgatatagcagtc

SEQ ID NO: 85 - DNA, 33 bp, Homo sapiens
ttcaaaacttactataacactgcggtaatcaag

SEQ ID NO: 86 - DNA, 33 bp, Homo sapiens
ttcaaaacttactatagcactgcggtaatcaag

SEQ ID NO: 87 - DNA, 33 bp, Homo sapiens
ggcattgacatctttacgagtgactttccatcc

SEQ ID NO: 88 - DNA, 33 bp, Homo sapiens
ggcattgacatctttatgagtgactttccatcc

SEQ ID NO: 89 - DNA, 33 bp, Homo sapiens
cccgtccttgagatcgaacctgtctttggggat

SEQ ID NO: 90 - DNA, 33 bp, Homo sapiens
cccgtccttgagatcggacctgtctttggggat

SEQ ID NO: 91 - DNA, 33 bp, Homo sapiens
tgtaagtcatgtatccgattagagacttctatc

SEQ ID NO: 92 - DNA, 33 bp, Homo sapiens
tgtaagtcatgtatcctattagagacttctatc

SEQ ID NO: 93 - DNA, 33 bp, Homo sapiens
gactaaaatattcctacgtgtattcaatcagtt

SEQ ID NO: 94 - DNA, 33 bp, Homo sapiens
gactaaaatattcctatgtgtattcaatcagtt

SEQ ID NO: 95 - DNA, 33 bp, Homo sapiens
gttttgaagatagagaacaaaagctgtgaagaa

SEQ ID NO: 96 - DNA, 33 bp, Homo sapiens
gttttgaagatagagagcaaaagctgtgaagaa

SEQ ID NO: 97 - DNA, 33 bp, Homo sapiens
tcataaagtgagtttgaatcctgaagaagccat

SEQ ID NO: 98 - DNA, 33 bp, Homo sapiens
tcataaagtgagtttggatcctgaagaagccat

SEQ ID NO: 99 - DNA, 33 bp, Homo sapiens
ctgccatgagttatgactaaccgagaaaaatgt

SEQ ID NO: 100 - DNA, 33 bp, Homo sapiens
ctgccatgagttatgattaaccgagaaaaatgt

SEQ ID NO: 101 - DNA, 33 bp, Homo sapiens
aatattgatgaatcctcaaaaaatgctagcaaa

SEQ ID NO: 102 - DNA, 33 bp, Homo sapiens
aatattgatgaatcctgaaaaaatgctagcaaa

SEQ ID NO: 103 - DNA, 33 bp, Homo sapiens
tgattctggggataaaactgtgaaccagataaa

SEQ ID NO: 104 - DNA, 33 bp, Homo sapiens
tgattctggggataaagctgtgaaccagataaa

SEQ ID NO: 105 - DNA, 33 bp, Homo sapiens
catcaactcaccaggagagtgagcactaagaac

SEQ ID NO: 106 - DNA, 33 bp, Homo sapiens
catcaactcaccaggatagtgagcactaagaac

SEQ ID NO: 107 - DNA, 33 bp, Homo sapiens
ggaaaagaagttactacagttttgcatagaaag

SEQ ID NO: 108 - DNA, 33 bp, Homo sapiens
ggaaaagaagttactatagttttgcatagaaag

SEQ ID NO: 109 - DNA, 33 bp, Homo sapiens
gacccctttggtgagacaatggtcttgaaagta

SEQ ID NO: 110 - DNA, 33 bp, Homo sapiens
gacccctttggtgagagaatggtcttgaaagta

SEQ ID NO: 111 - DNA, 33 bp, Homo sapiens
tttaccttttaacatacctggaattggaatgta

SEQ ID NO: 112 - DNA, 33 bp, Homo sapiens
tttaccttttaacatatctggaattggaatgta

SEQ ID NO: 113 - DNA, 33 bp, Homo sapiens
atacagtgtatatttcaattctatggctaaagt

SEQ ID NO: 114 - DNA, 33 bp, Homo sapiens
atacagtgtatatttcgattctatggctaaagt

SEQ ID NO: 115 - DNA, 33 bp, Homo sapiens
ccgcccaggagcgcgcaaaaccttctccacacc

SEQ ID NO: 116 - DNA, 33 bp, Homo sapiens
ccgcccaggagcgcgcgaaaccttctccacacc

SEQ ID NO: 117 - DNA, 33 bp, Homo sapiens
cttctgatttccccgagtcctaatgaagttgaa

SEQ ID NO: 118 - DNA, 33 bp, Homo sapiens
cttctgatttccccgattcctaatgaagttgaa

SEQ ID NO: 119 - DNA, 33 bp, Homo sapiens
cacatcattactgaaacggttgtagaatgttcc

SEQ ID NO: 120 - DNA, 33 bp, Homo sapiens
cacatcattactgaaatggttgtagaatgttcc

SEQ ID NO: 121 - DNA, 33 bp, Homo sapiens
ttgggagggtaagtaactggttgtgattgaaag

SEQ ID NO: 122 - DNA, 33 bp, Homo sapiens
ttgggagggtaagtaattggttgtgattgaaag

SEQ ID NO: 123 - DNA, 33 bp, Homo sapiens
gattcacaatctacaacaccagcccagcctatc

SEQ ID NO: 124 - DNA, 33 bp, Homo sapiens
gattcacaatctacaagaccagcccagcctatc

SEQ ID NO: 125 - DNA, 33 bp, Homo sapiens
aaactacacatagcaaacctcaaatttgaacag

SEQ ID NO: 126 - DNA, 33 bp, Homo sapiens
aaactacacatagcaatcctcaaatttgaacag

SEQ ID NO: 127 - DNA, 33 bp, Homo sapiens
aagtgcctcggatctccactttatctgttaata

SEQ ID NO: 128 - DNA, 33 bp, Homo sapiens
aagtgcctcggatctctactttatctgttaata

SEQ ID NO: 129 - DNA, 33 bp, Homo sapiens
tatcacgtgaatagacagtaacctttaataacc

SEQ ID NO: 130 - DNA, 33 bp, Homo sapiens
tatcacgtgaatagacggtaacctttaataacc

SEQ ID NO: 131 - DNA, 33 bp, Homo sapiens
ggtgtattgcccatggccagagaggaaattaag

SEQ ID NO: 132 - DNA, 33 bp, Homo sapiens
ggtgtattgcccatggtcagagaggaaattaag

SEQ ID NO: 133 - DNA, 33 bp, Homo sapiens
gccctttggtatcacaaaaaacctctaagccct

SEQ ID NO: 134 - DNA, 33 bp, Homo sapiens
gccctttggtatcacagaaaacctctaagccct

SEQ ID NO: 135 - DNA, 33 bp, Homo sapiens
ttaatctcaccatgaaaagtcaaagagaagagg

SEQ ID NO: 136 - DNA, 33 bp, Homo sapiens
ttaatctcaccatgaatagtcaaagagaagagg

SEQ ID NO: 137 - DNA, 33 bp, Homo sapiens
gtctcagtctctagaccaggtattctcagattc

SEQ ID NO: 138 - DNA, 33 bp, Homo sapiens
gtctcagtctctagacgaggtattctcagattc

SEQ ID NO: 139 - DNA, 33 bp, Homo sapiens
taaaacattttaaccccatctatgttttaaatt

SEQ ID NO: 140 - DNA, 33 bp, Homo sapiens
taaaacattttaacccgatctatgttttaaatt

SEQ ID NO: 141 - DNA, 33 bp, Homo sapiens
aaagggatataatagacagaaatggttgaaaaa

SEQ ID NO: 142 - DNA, 33 bp, Homo sapiens
aaagggatataatagagagaaatggttgaaaaa

SEQ ID NO: 143 - DNA, 33 bp, Homo sapiens
tgctgagtgaacacaaacagtaacctatgttct

SEQ ID NO: 144 - DNA, 33 bp, Homo sapiens
tgctgagtgaacacaagcagtaacctatgttct

SEQ ID NO: 145 - DNA, 33 bp, Homo sapiens
tatcatcagctacaccaaacactcaagtgtagc

SEQ ID NO: 146 - DNA, 33 bp, Homo sapiens
tatcatcagctacaccgaacactcaagtgtagc

SEQ ID NO: 147 - DNA, 33 bp, Homo sapiens
caaatcagggcaaaaagtcaacagcttctgccc

SEQ ID NO: 148 - DNA, 33 bp, Homo sapiens
caaatcagggcaaaaattcaacagcttctgccc

SEQ ID NO: 149 - DNA, 33 bp, Homo sapiens
cagttctggaatagagaatattgatttatgaca

SEQ ID NO: 150 - DNA, 33 bp, Homo sapiens
cagttctggaatagagcatattgatttatgaca

SEQ ID NO: 151 - DNA, 33 bp, Homo sapiens
cttttcactatcatcacgaaattcctttgcttc

SEQ ID NO: 152 - DNA, 33 bp, Homo sapiens
cttttcactatcatcatgaaattcctttgcttc

SEQ ID NO: 153 - DNA, 33 bp, Homo sapiens
gggtaaagataggtcacatgaagtaatctccgc

SEQ ID NO: 154 - DNA, 33 bp, Homo sapiens
gggtaaagataggtcagatgaagtaatctccgc

SEQ ID NO: 155 - DNA, 33 bp, Homo sapiens
ggctcctcgttgaagtcacctcaagcagcctga

SEQ ID NO: 156 - DNA, 33 bp, Homo sapiens
ggctcctcgttgaagttacctcaagcagcctga

SEQ ID NO: 157 - DNA, 33 bp, Homo sapiens
caaaatcatggctccacctctgtgatattttga

SEQ ID NO: 158 - DNA, 33 bp, Homo sapiens
caaaatcatggctccatctctgtgatattttga

SEQ ID NO: 159 - DNA, 33 bp, Homo sapiens
agctctgcagacctgacagatgtggcaaataca

SEQ ID NO: 160 - DNA, 33 bp, Homo sapiens
agctctgcagacctgatagatgtggcaaataca

SEQ ID NO: 161 - DNA, 33 bp, Homo sapiens
tacagttaggtaaagacagagtggtgagggtaa

SEQ ID NO: 162 - DNA, 33 bp, Homo sapiens
tacagttaggtaaagatagagtggtgagggtaa

SEQ ID NO: 163 - DNA, 33 bp, Homo sapiens
tctgtgctctcaataaccctatattcttgtgcg

SEQ ID NO: 164 - DNA, 33 bp, Homo sapiens
tctgtgctctcaataatcctatattcttgtgcg

SEQ ID NO: 165 - DNA, 33 bp, Homo sapiens
ttattaaacatgatacacaaagtacagtgaaaa

SEQ ID NO: 166 - DNA, 33 bp, Homo sapiens
ttattaaacatgatacccaaagtacagtgaaaa

SEQ ID NO: 167 - DNA, 33 bp, Homo sapiens
tacaaataggacatcacgaaaactgcatcaaat

SEQ ID NO: 168 - DNA, 33 bp, Homo sapiens
tacaaataggacatcatgaaaactgcatcaaat

SEQ ID NO: 169 - DNA, 33 bp, Homo sapiens
ggatgcccagtgcattgaaaatcgaaactctta

SEQ ID NO: 170 - DNA, 33 bp, Homo sapiens
ggatgcccagtgcatttaaaatcgaaactctta

SEQ ID NO: 171 - DNA, 33 bp, Homo sapiens
agtttgataatgtaaactggggaaagtgagtat

SEQ ID NO: 172 - DNA, 33 bp, Homo sapiens
agtttgataatgtaaattggggaaagtgagtat

SEQ ID NO: 173 - DNA, 33 bp, Homo sapiens
gaaatagccttacaacactgtgaattcaatcct

SEQ ID NO: 174 - DNA, 33 bp, Homo sapiens
gaaatagccttacaacgctgtgaattcaatcct

SEQ ID NO: 175 - DNA, 33 bp, Homo sapiens
aagatcacatgtgttacaccttaacgaaaaact

SEQ ID NO: 176 - DNA, 33 bp, Homo sapiens
aagatcacatgtgttataccttaacgaaaaact

SEQ ID NO: 177 - DNA, 33 bp, Homo sapiens
tagcagctcatacaagcataggtgatatcacaa

SEQ ID NO: 178 - DNA, 33 bp, Homo sapiens
tagcagctcatacaaggataggtgatatcacaa

SEQ ID NO: 179 - DNA, 33 bp, Homo sapiens
atcaatagcagattcaacttgcctttaccatgt

SEQ ID NO: 180 - DNA, 33 bp, Homo sapiens
atcaatagcagattcagcttgcctttaccatgt

SEQ ID NO: 181 - DNA, 33 bp, Homo sapiens
ttttacatccttgctcgcctgactttcatgaaa

SEQ ID NO: 182 - DNA, 33 bp, Homo sapiens
ttttacatccttgctctcctgactttcatgaaa

SEQ ID NO: 183 - DNA, 33 bp, Homo sapiens
gggctctttgcttgcaaaccagttcatcatacc

SEQ ID NO: 184 - DNA, 33 bp, Homo sapiens
gggctctttgcttgcagaccagttcatcatacc

SEQ ID NO: 185 - DNA, 33 bp, Homo sapiens
ggattatcagcatttaagccagagttgcaaatt

SEQ ID NO: 186 - DNA, 33 bp, Homo sapiens
ggattatcagcatttaggccagagttgcaaatt

SEQ ID NO: 187 - DNA, 33 bp, Homo sapiens
acccaatttgtttttaccagttggaacactttt

SEQ ID NO: 188 - DNA, 33 bp, Homo sapiens
acccaatttgtttttatcagttggaacactttt

SEQ ID NO: 189 - DNA, 33 bp, Homo sapiens
cagattgctgaacttcacaaaggctagagagct

SEQ ID NO: 190 - DNA, 33 bp, Homo sapiens
cagattgctgaacttcgcaaaggctagagagct

SEQ ID NO: 191 - DNA, 33 bp, Homo sapiens
taagcacaatattgaactataaaagacatcttg

SEQ ID NO: 192 - DNA, 33 bp, Homo sapiens
taagcacaatattgaagtataaaagacatcttg

SEQ ID NO: 193 - DNA, 33 bp, Homo sapiens
atggttccaagttgccaacaacaaatgtcgcac

SEQ ID NO: 194 - DNA, 33 bp, Homo sapiens
atggttccaagttgcccacaacaaatgtcgcac

SEQ ID NO: 195 - DNA, 33 bp, Homo sapiens
cactctaatgcacatgcacaataggcaaggcag

SEQ ID NO: 196 - DNA, 33 bp, Homo sapiens
cactctaatgcacatggacaataggcaaggcag

SEQ ID NO: 197 - DNA, 33 bp, Homo sapiens
caactatttaactgtacctttgtcgtacaaaaa

SEQ ID NO: 198 - DNA, 33 bp, Homo sapiens
caactatttaactgtatctttgtcgtacaaaaa

SEQ ID NO: 199 - DNA, 33 bp, Homo sapiens
ctattagttactttccagactctcttcctcctc

SEQ ID NO: 200 - DNA, 33 bp, Homo sapiens
ctattagttactttcccgactctcttcctcctc

SEQ ID NO: 201 - DNA, 33 bp, Homo sapiens
taaacactctcatggaacatcagtgtcagacca

SEQ ID NO: 202 - DNA, 33 bp, Homo sapiens
taaacactctcatggagcatcagtgtcagacca

SEQ ID NO: 203 - DNA, 33 bp, Homo sapiens
cctggctctaaattcccccgtattttgtaatct

SEQ ID NO: 204 - DNA, 33 bp, Homo sapiens
cctggctctaaattccgccgtattttgtaatct

SEQ ID NO: 205 - DNA, 33 bp, Homo sapiens
ggtacttgactccagaagactacagtgataatt

SEQ ID NO: 206 - DNA, 33 bp, Homo sapiens
ggtacttgactccagatgactacagtgataatt

SEQ ID NO: 207 - DNA, 33 bp, Homo sapiens
ccctttgacagcaaaacctggcctttcctgaat

SEQ ID NO: 208 - DNA, 33 bp, Homo sapiens
ccctttgacagcaaaatctggcctttcctgaat

SEQ ID NO: 209 - DNA, 33 bp, Homo sapiens
atcatctgagaaaaaaatatagacccagaacaa

SEQ ID NO: 210 - DNA, 33 bp, Homo sapiens
atcatctgagaaaaaagtatagacccagaacaa

SEQ ID NO: 211 - DNA, 33 bp, Homo sapiens
caaatgagtgttgtgcacttaaggaaattggct

SEQ ID NO: 212 - DNA, 33 bp, Homo sapiens
caaatgagtgttgtgcccttaaggaaattggct

SEQ ID NO: 213 - DNA, 33 bp, Homo sapiens
gaagagatgtgggggcagaaagacaggaaatag

SEQ ID NO: 214 - DNA, 33 bp, Homo sapiens
gaagagatgtgggggcggaaagacaggaaatag

SEQ ID NO: 215 - DNA, 33 bp, Homo sapiens
tagtgaggctcacagcaagttaggtagatggga

SEQ ID NO: 216 - DNA, 33 bp, Homo sapiens
tagtgaggctcacagcgagttaggtagatggga

SEQ ID NO: 217 - DNA, 33 bp, Homo sapiens
actatcttaagtgcaccgaataatcaacaccag

SEQ ID NO: 218 - DNA, 33 bp, Homo sapiens
actatcttaagtgcactgaataatcaacaccag

SEQ ID NO: 219 - DNA, 33 bp, Homo sapiens
aaaaataataagcctaccttatctctctacatg

SEQ ID NO: 220 - DNA, 33 bp, Homo sapiens
aaaaataataagcctatcttatctctctacatg

SEQ ID NO: 221 - DNA, 33 bp, Homo sapiens
aattgtagctctcacaataaagctgggtgaaaa

SEQ ID NO: 222 - DNA, 33 bp, Homo sapiens
aattgtagctctcacattaaagctgggtgaaaa

SEQ ID NO: 223 - DNA, 33 bp, Homo sapiens
acaaagcctccttttgaatggattaggaaaaca

SEQ ID NO: 224 - DNA, 33 bp, Homo sapiens
acaaagcctccttttgcatggattaggaaaaca

SEQ ID NO: 225 - DNA, 33 bp, Homo sapiens
gctctaaagaaacagcacttgcctgggcttggc

SEQ ID NO: 226 - DNA, 33 bp, Homo sapiens
gctctaaagaaacagcgcttgcctgggcttggc

SEQ ID NO: 227 - DNA, 33 bp, Homo sapiens
cctcagtgacgctctaacatagcttgttatagt

SEQ ID NO: 228 - DNA, 33 bp, Homo sapiens
cctcagtgacgctctaccatagcttgttatagt

SEQ ID NO: 229 - DNA, 33 bp, Homo sapiens
agcactgacttcaatacgtccatcaattttcca

SEQ ID NO: 230 - DNA, 33 bp, Homo sapiens
agcactgacttcaatatgtccatcaattttcca

SEQ ID NO: 231 - DNA, 33 bp, Homo sapiens
ccagggctagctctggacatttcaagagatgta

SEQ ID NO: 232 - DNA, 33 bp, Homo sapiens
ccagggctagctctgggcatttcaagagatgta

SEQ ID NO: 233 - DNA, 33 bp, Homo sapiens
aaggtcaccaagttgaacatgtatatggcacag

SEQ ID NO: 234 - DNA, 33 bp, Homo sapiens
aaggtcaccaagttgagcatgtatatggcacag

SEQ ID NO: 235 - DNA, 33 bp, Homo sapiens
tgttttcattatgctaagtacaagattgtgcta

SEQ ID NO: 236 - DNA, 33 bp, Homo sapiens
tgttttcattatgctatgtacaagattgtgcta

SEQ ID NO: 237 - DNA, 33 bp, Homo sapiens
tgcattaaaatctgtaccaggaatcacttctgc

SEQ ID NO: 238 - DNA, 33 bp, Homo sapiens
tgcattaaaatctgtagcaggaatcacttctgc

SEQ ID NO: 239 - DNA, 33 bp, Homo sapiens
acaggatctagtgctcaattgcaagcaggtgac

SEQ ID NO: 240 - DNA, 33 bp, Homo sapiens
acaggatctagtgctcgattgcaagcaggtgac

SEQ ID NO: 241 - DNA, 33 bp, Homo sapiens
ctgaagtccttgctgccagttcaaacttgttca

SEQ ID NO: 242 - DNA, 33 bp, Homo sapiens
ctgaagtccttgctgctagttcaaacttgttca

SEQ ID NO: 243 - DNA, 33 bp, Homo sapiens
agaggaaatgatgtgcaaatgagaaaattttaa

SEQ ID NO: 244 - DNA, 33 bp, Homo sapiens
agaggaaatgatgtgcgaatgagaaaattttaa

SEQ ID NO: 245 - DNA, 33 bp, Homo sapiens
tttcaattatgctccactgacataacagctgtt

SEQ ID NO: 246 - DNA, 33 bp, Homo sapiens
tttcaattatgctccattgacataacagctgtt

SEQ ID NO: 247 - DNA, 33 bp, Homo sapiens
tgttaaataaataaaccaggcagagaaagacaa

SEQ ID NO: 248 - DNA, 33 bp, Homo sapiens
tgttaaataaataaactaggcagagaaagacaa

SEQ ID NO: 249 - DNA, 33 bp, Homo sapiens
ttaaactggataccgacacccagattgaggtga

SEQ ID NO: 250 - DNA, 33 bp, Homo sapiens
ttaaactggataccgagacccagattgaggtga

SEQ ID NO: 251 - DNA, 33 bp, Homo sapiens
aaaatcaaacaggagaacaacgttacacagaat

SEQ ID NO: 252 - DNA, 33 bp, Homo sapiens
aaaatcaaacaggagagcaacgttacacagaat

SEQ ID NO: 253 - DNA, 33 bp, Homo sapiens
agggggttgtgaacccaagggaactgctctgcc

SEQ ID NO: 254 - DNA, 33 bp, Homo sapiens
agggggttgtgaacccgagggaactgctctgcc

SEQ ID NO: 255 - DNA, 33 bp, Homo sapiens
aatctaacacagctccaagaattttcttctggt

SEQ ID NO: 256 - DNA, 33 bp, Homo sapiens
aatctaacacagctccgagaattttcttctggt

SEQ ID NO: 257 - DNA, 33 bp, Homo sapiens
ttcaaaaaaagtgtaccatttccatctgatcag

SEQ ID NO: 258 - DNA, 33 bp, Homo sapiens
ttcaaaaaaagtgtactatttccatctgatcag

SEQ ID NO: 259 - DNA, 33 bp, Homo sapiens
gttatgtatcacttaaggacagagacacattat

SEQ ID NO: 260 - DNA, 33 bp, Homo sapiens
gttatgtatcacttaatgacagagacacattat

SEQ ID NO: 261 - DNA, 33 bp, Homo sapiens
tataacaacgtagtgaaatagaagttatgtgaa

SEQ ID NO: 262 - DNA, 33 bp, Homo sapiens
tataacaacgtagtgagatagaagttatgtgaa

SEQ ID NO: 263 - DNA, 33 bp, Homo sapiens
tgtttaaacttatgcagtctttgggccagtctt

SEQ ID NO: 264 - DNA, 33 bp, Homo sapiens
tgtttaaacttatgcattctttgggccagtctt

SEQ ID NO: 265 - DNA, 33 bp, Homo sapiens
agtcactgcactaatgcaaaggcaagaagaaca

SEQ ID NO: 266 - DNA, 33 bp, Homo sapiens
agtcactgcactaatgtaaaggcaagaagaaca

SEQ ID NO: 267 - DNA, 33 bp, Homo sapiens
ccaaatgatctgccaaccctctgaataaccagc

SEQ ID NO: 268 - DNA, 33 bp, Homo sapiens
ccaaatgatctgccaatcctctgaataaccagc

SEQ ID NO: 269 - DNA, 33 bp, Homo sapiens
ctctaatgatgtaaaacgcctcagacatcgtta

SEQ ID NO: 270 - DNA, 33 bp, Homo sapiens
ctctaatgatgtaaaaggcctcagacatcgtta

SEQ ID NO: 271 - DNA, 33 bp, Homo sapiens
aaggctgctcatttgacggatgaattactccat

SEQ ID NO: 272 - DNA, 33 bp, Homo sapiens
aaggctgctcatttgatggatgaattactccat

SEQ ID NO: 273 - DNA, 33 bp, Homo sapiens
ggtaggacattgcttaacttcagtgaatccaca

SEQ ID NO: 274 - DNA, 33 bp, Homo sapiens
ggtaggacattgcttagcttcagtgaatccaca

SEQ ID NO: 275 - DNA, 33 bp, Homo sapiens
tcatctgtgaatgaagaatgtcaatattcagga

SEQ ID NO: 276 - DNA, 33 bp, Homo sapiens
tcatctgtgaatgaaggatgtcaatattcagga

SEQ ID NO: 277 - DNA, 33 bp, Homo sapiens
cccaccactccacaacaagccactaggagtttc

SEQ ID NO: 278 - DNA, 33 bp, Homo sapiens
cccaccactccacaacgagccactaggagtttc

SEQ ID NO: 279 - DNA, 33 bp, Homo sapiens
ttacatatcagttgctaagaatatcttcttcca

SEQ ID NO: 280 - DNA, 33 bp, Homo sapiens
ttacatatcagttgctgagaatatcttcttcca

SEQ ID NO: 281 - DNA, 33 bp, Homo sapiens
agagtgtgcgctcaagcaggaaagaagtagcat

SEQ ID NO: 282 - DNA, 33 bp, Homo sapiens
agagtgtgcgctcaagtaggaaagaagtagcat

SEQ ID NO: 283 - DNA, 33 bp, Homo sapiens
cagtgccataggaagaagtgctcaagaagcgtg

SEQ ID NO: 284 - DNA, 33 bp, Homo sapiens
cagtgccataggaagatgtgctcaagaagcgtg

SEQ ID NO: 285 - DNA, 33 bp, Homo sapiens
aagatttttaagcagcagagtaacatggttcag

SEQ ID NO: 286 - DNA, 33 bp, Homo sapiens
aagatttttaagcagcggagtaacatggttcag

SEQ ID NO: 287 - DNA, 33 bp, Homo sapiens
aggctggggactagtgcatcatcgcttaggatt

SEQ ID NO: 288 - DNA, 33 bp, Homo sapiens
aggctggggactagtggatcatcgcttaggatt

SEQ ID NO: 289 - DNA, 33 bp, Homo sapiens
caaataagcaacttacggaaggtcatgtgactc

SEQ ID NO: 290 - DNA, 33 bp, Homo sapiens
caaataagcaacttactgaaggtcatgtgactc

SEQ ID NO: 291 - DNA, 33 bp, Homo sapiens
tcctgggaacagtaacaccccagtaacactgag

SEQ ID NO: 292 - DNA, 33 bp, Homo sapiens
tcctgggaacagtaacgccccagtaacactgag

SEQ ID NO: 293 - DNA, 33 bp, Homo sapiens
aactgagactgccaaacagataatctagaatcc

SEQ ID NO: 294 - DNA, 33 bp, Homo sapiens
aactgagactgccaaagagataatctagaatcc

SEQ ID NO: 295 - DNA, 33 bp, Homo sapiens
taaggacacagattcacgggtgagctttagggg

SEQ ID NO: 296 - DNA, 33 bp, Homo sapiens
taaggacacagattcatgggtgagctttagggg

SEQ ID NO: 297 - DNA, 33 bp, Homo sapiens
tttctaataaatacacaattctatgtcaaattc

SEQ ID NO: 298 - DNA, 33 bp, Homo sapiens
tttctaataaatacacgattctatgtcaaattc

SEQ ID NO: 299 - DNA, 33 bp, Homo sapiens
acctgcctggtgtctacgatctgagactcttct

SEQ ID NO: 300 - DNA, 33 bp, Homo sapiens
acctgcctggtgtctatgatctgagactcttct

SEQ ID NO: 301 - DNA, 33 bp, Homo sapiens
tagggaagaaagcctacggtggttatggaggga

SEQ ID NO: 302 - DNA, 33 bp, Homo sapiens
tagggaagaaagcctatggtggttatggaggga

SEQ ID NO: 303 - DNA, 33 bp, Homo sapiens
cagatccaagtgtgttgaaaaatagtgtcccag

SEQ ID NO: 304 - DNA, 33 bp, Homo sapiens
cagatccaagtgtgtttaaaaatagtgtcccag

SEQ ID NO: 305 - DNA, 33 bp, Homo sapiens
gagttcccaaccatgacgggatgtgggtagaac

SEQ ID NO: 306 - DNA, 33 bp, Homo sapiens
gagttcccaaccatgatgggatgtgggtagaac

SEQ ID NO: 307 - DNA, 33 bp, Homo sapiens
ggatggagttccaggcactcttcaggtagtagc

SEQ ID NO: 308 - DNA, 33 bp, Homo sapiens
ggatggagttccaggcgctcttcaggtagtagc

SEQ ID NO: 309 - DNA, 33 bp, Homo sapiens
gttatttacatagttacaatgctctttcaaaca

SEQ ID NO: 310 - DNA, 33 bp, Homo sapiens
gttatttacatagttataatgctctttcaaaca

SEQ ID NO: 311 - DNA, 33 bp, Homo sapiens
atttcaggaaattttgcaattgaggcacaagga

SEQ ID NO: 312 - DNA, 33 bp, Homo sapiens
atttcaggaaattttgtaattgaggcacaagga

SEQ ID NO: 313 - DNA, 33 bp, Homo sapiens
gtgatggcccagaaaagagtagtgaagccaatt

SEQ ID NO: 314 - DNA, 33 bp, Homo sapiens
gtgatggcccagaaaatagtagtgaagccaatt

SEQ ID NO: 315 - DNA, 33 bp, Homo sapiens
taatttgagagacaataaaacacacaaaaaatg

SEQ ID NO: 316 - DNA, 33 bp, Homo sapiens
taatttgagagacaatcaaacacacaaaaaatg

SEQ ID NO: 317 - DNA, 33 bp, Homo sapiens
tctacctctcgtcttccctaatcagctcctcct

SEQ ID NO: 318 - DNA, 33 bp, Homo sapiens
tctacctctcgtcttctctaatcagctcctcct

SEQ ID NO: 319 - DNA, 33 bp, Homo sapiens
agtccatttgtgtacaatcagaacctttcaacc

SEQ ID NO: 320 - DNA, 33 bp, Homo sapiens
agtccatttgtgtacagtcagaacctttcaacc

SEQ ID NO: 321 - DNA, 33 bp, Homo sapiens
atgctgtgttttccacaatactggtactaccgg

SEQ ID NO: 322 - DNA, 33 bp, Homo sapiens
atgctgtgttttccacgatactggtactaccgg

SEQ ID NO: 323 - DNA, 33 bp, Homo sapiens
aaggatatttggacacacttaatatcttttgga

SEQ ID NO: 324 - DNA, 33 bp, Homo sapiens
aaggatatttggacacgcttaatatcttttgga

SEQ ID NO: 325 - DNA, 33 bp, Homo sapiens
atggcgcatattgaaacgaaaaggatatagaaa

SEQ ID NO: 326 - DNA, 33 bp, Homo sapiens
atggcgcatattgaaatgaaaaggatatagaaa

SEQ ID NO: 327 - DNA, 33 bp, Homo sapiens
agccctgaaaatcagcacaacctcggacatcca

SEQ ID NO: 328 - DNA, 33 bp, Homo sapiens
agccctgaaaatcagcgcaacctcggacatcca

SEQ ID NO: 329 - DNA, 33 bp, Homo sapiens
gggccacatgctgatacgtgagaggcggtgggg

SEQ ID NO: 330 - DNA, 33 bp, Homo sapiens
gggccacatgctgataggtgagaggcggtgggg

SEQ ID NO: 331 - DNA, 33 bp, Homo sapiens
cttgaactgagtaggccaaaaatttgctttcct

SEQ ID NO: 332 - DNA, 33 bp, Homo sapiens
cttgaactgagtaggctaaaaatttgctttcct

SEQ ID NO: 333 - DNA, 33 bp, Homo sapiens
gtactcttatcaggcaagacatttccatggcct

SEQ ID NO: 334 - DNA, 33 bp, Homo sapiens
gtactcttatcaggcaggacatttccatggcct

SEQ ID NO: 335 - DNA, 33 bp, Homo sapiens
aaatcatattccaaataagcatcaaaaataata

SEQ ID NO: 336 - DNA, 33 bp, Homo sapiens
aaatcatattccaaatgagcatcaaaaataata

SEQ ID NO: 337 - DNA, 33 bp, Homo sapiens
tttgcaatggtgttctcaaaaattagaatgtta

SEQ ID NO: 338 - DNA, 33 bp, Homo sapiens
tttgcaatggtgttcttaaaaattagaatgtta

SEQ ID NO: 339 - DNA, 33 bp, Homo sapiens
gcacccaaagataaatcaatgatgttacgaagc

SEQ ID NO: 340 - DNA, 33 bp, Homo sapiens
gcacccaaagataaatgaatgatgttacgaagc

SEQ ID NO: 341 - DNA, 33 bp, Homo sapiens
agggccttatgtgttacgttaagcaccaggaac

SEQ ID NO: 342 - DNA, 33 bp, Homo sapiens
agggccttatgtgttatgttaagcaccaggaac

SEQ ID NO: 343 - DNA, 33 bp, Homo sapiens
atccctgcaagaccatcaatgtaatgaacagaa

SEQ ID NO: 344 - DNA, 33 bp, Homo sapiens
atccctgcaagaccattaatgtaatgaacagaa

SEQ ID NO: 345 - DNA, 33 bp, Homo sapiens
acgtaggacgccggcacagaaggagccgggtgg

SEQ ID NO: 346 - DNA, 33 bp, Homo sapiens
acgtaggacgccggcagagaaggagccgggtgg

SEQ ID NO: 347 - DNA, 33 bp, Homo sapiens
catgtccaaatgactacctacagttcaaactgc

SEQ ID NO: 348 - DNA, 33 bp, Homo sapiens
catgtccaaatgactatctacagttcaaactgc

SEQ ID NO: 349 - DNA, 33 bp, Homo sapiens
agagaaaattaaaacagtgttggccaagaatgc

SEQ ID NO: 350 - DNA, 33 bp, Homo sapiens
agagaaaattaaaacattgttggccaagaatgc

SEQ ID NO: 351 - DNA, 33 bp, Homo sapiens
ctgtaaacttctatacgactttgtaccacccat

SEQ ID NO: 352 - DNA, 33 bp, Homo sapiens
ctgtaaacttctatactactttgtaccacccat

SEQ ID NO: 353 - DNA, 33 bp, Homo sapiens
aattttgaggtgatacaagcatttagtccaaag

SEQ ID NO: 354 - DNA, 33 bp, Homo sapiens
aattttgaggtgatactagcatttagtccaaag

SEQ ID NO: 355 - DNA, 33 bp, Homo sapiens
gtgatggttggagaaacgggcacctggctttaa

SEQ ID NO: 356 - DNA, 33 bp, Homo sapiens
gtgatggttggagaaatgggcacctggctttaa

SEQ ID NO: 357 - DNA, 33 bp, Homo sapiens
gagttgtcaaagaggactcacagcagaggaaaa

SEQ ID NO: 358 - DNA, 33 bp, Homo sapiens
gagttgtcaaagaggagtcacagcagaggaaaa

SEQ ID NO: 359 - DNA, 33 bp, Homo sapiens
tgctgaacaaaataataaccgtctcaaaaataa

SEQ ID NO: 360 - DNA, 33 bp, Homo sapiens
tgctgaacaaaataatcaccgtctcaaaaataa

SEQ ID NO: 361 - DNA, 33 bp, Homo sapiens
catgatgtcctaacaaacaaattatcactttaa

SEQ ID NO: 362 - DNA, 33 bp, Homo sapiens
catgatgtcctaacaagcaaattatcactttaa

SEQ ID NO: 363 - DNA, 33 bp, Homo sapiens
agtctgagtttaatcaccctgcaatactgtcct

SEQ ID NO: 364 - DNA, 33 bp, Homo sapiens
agtctgagtttaatcatcctgcaatactgtcct

SEQ ID NO: 365 - DNA, 33 bp, Homo sapiens
ggaagagtccaaatgtcaaatatggttccagga

SEQ ID NO: 366 - DNA, 33 bp, Homo sapiens
ggaagagtccaaatgttaaatatggttccagga

SEQ ID NO: 367 - DNA, 33 bp, Homo sapiens
gccaaaatttgggcgcagattccaccacttggt

SEQ ID NO: 368 - DNA, 33 bp, Homo sapiens
gccaaaatttgggcgccgattccaccacttggt

SEQ ID NO: 369 - DNA, 33 bp, Homo sapiens
actctattccattggaaacaaacctaggttgta

SEQ ID NO: 370 - DNA, 33 bp, Homo sapiens
actctattccattggagacaaacctaggttgta

SEQ ID NO: 371 - DNA, 33 bp, Homo sapiens
ctgtcaaatcaactcaccttcaacaaaggagtc

SEQ ID NO: 372 - DNA, 33 bp, Homo sapiens
ctgtcaaatcaactcatcttcaacaaaggagtc

SEQ ID NO: 373 - DNA, 33 bp, Homo sapiens
gatttcccttgttgaaacttatacacacttacc

SEQ ID NO: 374 - DNA, 33 bp, Homo sapiens
gatttcccttgttgaaccttatacacacttacc

SEQ ID NO: 375 - DNA, 33 bp, Homo sapiens
caggatattggacacaactgaagagtaaattca

SEQ ID NO: 376 - DNA, 33 bp, Homo sapiens
caggatattggacacatctgaagagtaaattca

SEQ ID NO: 377 - DNA, 33 bp, Homo sapiens
tatttttcgttttcagaacattcagttggtatg

SEQ ID NO: 378 - DNA, 33 bp, Homo sapiens
tatttttcgttttcaggacattcagttggtatg

SEQ ID NO: 379 - DNA, 33 bp, Homo sapiens
ctaggcttgaatgagaaaataacgatacctttc

SEQ ID NO: 380 - DNA, 33 bp, Homo sapiens
ctaggcttgaatgagagaataacgatacctttc

SEQ ID NO: 381 - DNA, 33 bp, Homo sapiens
gcacctcctctgtccaagatatgatgccaagta

SEQ ID NO: 382 - DNA, 33 bp, Homo sapiens
gcacctcctctgtccaggatatgatgccaagta

SEQ ID NO: 383 - DNA, 33 bp, Homo sapiens; identical to SEQ ID NO: 349

SEQ ID NO: 384 - DNA, 33 bp, Homo sapiens; identical to SEQ ID NO: 350

SEQ ID NO: 385 - DNA, 33 bp, Homo sapiens
gcatgtttctaaaaacacttcaggaatccagag

SEQ ID NO: 386 - DNA, 33 bp, Homo sapiens
gcatgtttctaaaaacgcttcaggaatccagag

SEQ ID NO: 387 - DNA, 33 bp, Homo sapiens
cacatgtgggtcgtcaactgccatcaggagtag

SEQ ID NO: 388 - DNA, 33 bp, Homo sapiens
cacatgtgggtcgtcagctgccatcaggagtag

SEQ ID NO: 389 - DNA, 33 bp, Homo sapiens; identical to SEQ ID NO: 355

SEQ ID NO: 390 - DNA, 33 bp, Homo sapiens; identical to SEQ ID NO: 356

SEQ ID NO: 391 - DNA, 33 bp, Homo sapiens
aactctggtgttgacccagcgggtagcatgaga

SEQ ID NO: 392 - DNA, 33 bp, Homo sapiens
aactctggtgttgacctagcgggtagcatgaga

SEQ ID NO: 393 - DNA, 33 bp, Homo sapiens
gtaaatgtttcgagtacgttattgggctccatt

SEQ ID NO: 394 - DNA, 33 bp, Homo sapiens
gtaaatgtttcgagtatgttattgggctccatt

SEQ ID NO: 395 - DNA, 33 bp, Homo sapiens
gaagtgttagtgatgacggacagaaatagacaa

SEQ ID NO: 396 - DNA, 33 bp, Homo sapiens
gaagtgttagtgatgatggacagaaatagacaa

SEQ ID NO: 397 - DNA, 33 bp, Homo sapiens
agatacaaaccagattcaaaggatctttaatat

SEQ ID NO: 398 - DNA, 33 bp, Homo sapiens
agatacaaaccagattgaaaggatctttaatat

SEQ ID NO: 399 - DNA, 33 bp, Homo sapiens
gcgttagaattagagacgagcctgtgacgcttg

SEQ ID NO: 400 - DNA, 33 bp, Homo sapiens
gcgttagaattagagaggagcctgtgacgcttg

SEQ ID NO: 401 - DNA, 33 bp, Homo sapiens
atgcaaatggatccaacacttggttagccaata

SEQ ID NO: 402 - DNA, 33 bp, Homo sapiens
atgcaaatggatccaatacttggttagccaata

SEQ ID NO: 403 - DNA, 33 bp, Homo sapiens; identical to SEQ ID NO: 363

SEQ ID NO: 404 - DNA, 33 bp, Homo sapiens; identical to SEQ ID NO: 364

SEQ ID NO: 405 - DNA, 33 bp, Homo sapiens
tcacatttcaatggtacgaaaagtggcttcgta

SEQ ID NO: 406 - DNA, 33 bp, Homo sapiens
tcacatttcaatggtaggaaaagtggcttcgta

SEQ ID NO: 407 - DNA, 33 bp, Homo sapiens
taaatggtgcctctaaaataaagggaaataaaa

SEQ ID NO: 408 - DNA, 33 bp, Homo sapiens
taaatggtgcctctaacataaagggaaataaaa

SEQ ID NO: 409 - DNA, 33 bp, Homo sapiens
agcactttgctcttccgcaccaatgcagtacag

SEQ ID NO: 410 - DNA, 33 bp, Homo sapiens
agcactttgctcttcctcaccaatgcagtacag

SEQ ID NO: 411 - DNA, 33 bp, Homo sapiens
cttagctctttgcttacggtgttgccaaattcc

SEQ ID NO: 412 - DNA, 33 bp, Homo sapiens
cttagctctttgcttatggtgttgccaaattcc

SEQ ID NO: 413 - DNA, 33 bp, Homo sapiens
cagatggtcacttcatcagcataaacactaagc

SEQ ID NO: 414 - DNA, 33 bp, Homo sapiens
cagatggtcacttcattagcataaacactaagc

SEQ ID NO: 415 - DNA, 33 bp, Homo sapiens
taatgatgggactcagacataaggaaaaagcaa

SEQ ID NO: 416 - DNA, 33 bp, Homo sapiens
taatgatgggactcaggcataaggaaaaagcaa

SEQ ID NO: 417 - DNA, 33 bp, Homo sapiens
ctaagctaactgaaaaaattggcatatcacagg

SEQ ID NO: 418 - DNA, 33 bp, Homo sapiens
ctaagctaactgaaaagattggcatatcacagg

SEQ ID NO: 419 - DNA, 33 bp, Homo sapiens
agggctgattatgttccaccaagtcactatgga

SEQ ID NO: 420 - DNA, 33 bp, Homo sapiens
agggctgattatgttctaccaagtcactatgga

SEQ ID NO: 421 - DNA, 33 bp, Homo sapiens
tgagaactacccaactaagccaaagtaacccac

SEQ ID NO: 422 - DNA, 33 bp, Homo sapiens
tgagaactacccaactgagccaaagtaacccac

SEQ ID NO: 423 - DNA, 33 bp, Homo sapiens
atatttcagctccacaatgaaatcattttatcc

SEQ ID NO: 424 - DNA, 33 bp, Homo sapiens
atatttcagctccacagtgaaatcattttatcc

SEQ ID NO: 425 - DNA, 33 bp, Homo sapiens
caggaactgccttttacgtctgcaaataaactc

SEQ ID NO: 426 - DNA, 33 bp, Homo sapiens
caggaactgccttttatgtctgcaaataaactc

SEQ ID NO: 427 - DNA, 33 bp, Homo sapiens
tggtctgaaattgagacgcttattgaatgctta

SEQ ID NO: 428 - DNA, 33 bp, Homo sapiens
tggtctgaaattgagatgcttattgaatgctta

SEQ ID NO: 429 - DNA, 33 bp, Homo sapiens
tggaggaaaacctcaccgacttggcaacatttg

SEQ ID NO: 430 - DNA, 33 bp, Homo sapiens
tggaggaaaacctcactgacttggcaacatttg

SEQ ID NO: 431 - DNA, 33 bp, Homo sapiens
accacaagcaacggacaagtaggggaagagtcg

SEQ ID NO: 432 - DNA, 33 bp, Homo sapiens
accacaagcaacggactagtaggggaagagtcg

SEQ ID NO: 433 - DNA, 33 bp, Homo sapiens
atacttaatttttccaatatggtctaggttaga

SEQ ID NO: 434 - DNA, 33 bp, Homo sapiens
atacttaatttttccagtatggtctaggttaga

SEQ ID NO: 435 - DNA, 33 bp, Homo sapiens; identical to SEQ ID NO: 345

SEQ ID NO: 436 - DNA, 33 bp, Homo sapiens; identical to SEQ ID NO: 346

SEQ ID NO: 437 - DNA, 33 bp, Homo sapiens; identical to SEQ ID NO: 359

SEQ ID NO: 438 - DNA, 33 bp, Homo sapiens; identical to SEQ ID NO: 360

SEQ ID NO: 439 - DNA, 33 bp, Homo sapiens; identical to SEQ ID NO: 365

SEQ ID NO: 440 - DNA, 33 bp, Homo sapiens; identical to SEQ ID NO: 366

SEQ ID NO: 441 - DNA, 33 bp, Homo sapiens; identical to SEQ ID NO: 371

SEQ ID NO: 442 - DNA, 33 bp, Homo sapiens; identical to SEQ ID NO: 372

SEQ ID NO: 443 - DNA, 33 bp, Homo sapiens
agactcaaagatagagaccaaaaaagtgacact

SEQ ID NO: 444 - DNA, 33 bp, Homo sapiens
agactcaaagatagagcccaaaaaagtgacact

SEQ ID NO: 445 - DNA, 33 bp, Homo sapiens
ttctagtgcttcagaaggaataaaaggtcatag

SEQ ID NO: 446 - DNA, 33 bp, Homo sapiens
ttctagtgcttcagaatgaataaaaggtcatag

SEQ ID NO: 447 - DNA, 33 bp, Homo sapiens
gtagcacagctgtaggaatattgactcctcttc

SEQ ID NO: 448 - DNA, 33 bp, Homo sapiens
gtagcacagctgtagggatattgactcctcttc

SEQ ID NO: 449 - DNA, 33 bp, Homo sapiens
caatgttaagcacagacagttgagtagcttctt

SEQ ID NO: 450 - DNA, 33 bp, Homo sapiens
caatgttaagcacagagagttgagtagcttctt

SEQ ID NO: 451 - DNA, 33 bp, Homo sapiens
tatttgtgaattgccacgccaaaagaaaataac

SEQ ID NO: 452 - DNA, 33 bp, Homo sapiens
tatttgtgaattgccatgccaaaagaaaataac

SEQ ID NO: 453 - DNA, 33 bp, Homo sapiens
ttcctcctaggagtgacagtgaaataatctgaa

SEQ ID NO: 454 - DNA, 33 bp, Homo sapiens
ttcctcctaggagtgagagtgaaataatctgaa

SEQ ID NO: 455 - DNA, 33 bp, Homo sapiens
attcattattgccaaaaatttctagaggacatg

SEQ ID NO: 456 - DNA, 33 bp, Homo sapiens
attcattattgccaaagatttctagaggacatg

SEQ ID NO: 457 - DNA, 33 bp, Homo sapiens
ctgtcgtgtttaggaacgtccacctattactgt

SEQ ID NO: 458 - DNA, 33 bp, Homo sapiens
ctgtcgtgtttaggaatgtccacctattactgt

SEQ ID NO: 459 - DNA, 33 bp, Homo sapiens
tcattacagcagctaacgtaggctaagcccaca

SEQ ID NO: 460 - DNA, 33 bp, Homo sapiens
tcattacagcagctaatgtaggctaagcccaca

SEQ ID NO: 461 - DNA, 33 bp, Homo sapiens
gaaactgaggcacagtaaactaattttcccaag

SEQ ID NO: 462 - DNA, 33 bp, Homo sapiens
gaaactgaggcacagtgaactaattttcccaag

SEQ ID NO: 463 - DNA, 33 bp, Homo sapiens
tgagtgagttaccatacaacctgagctgcccat

SEQ ID NO: 464 - DNA, 33 bp, Homo sapiens
tgagtgagttaccatagaacctgagctgcccat

SEQ ID NO: 465 - DNA, 33 bp, Homo sapiens
cccttatctttgctccagactaacctgtggctg

SEQ ID NO: 466 - DNA, 33 bp, Homo sapiens
cccttatctttgctccggactaacctgtggctg

SEQ ID NO: 467 - DNA, 33 bp, Homo sapiens
ttgcttacagggaaacactatctgagacaagct

SEQ ID NO: 468 - DNA, 33 bp, Homo sapiens
ttgcttacagggaaacgctatctgagacaagct

SEQ ID NO: 469 - DNA, 33 bp, Homo sapiens
ctaccaacactgtgagaaaatttggatatggaa

SEQ ID NO: 470 - DNA, 33 bp, Homo sapiens
ctaccaacactgtgaggaaatttggatatggaa

SEQ ID NO: 471 - DNA, 33 bp, Homo sapiens
cctataggaaacaccaatagtgacctacacagc

SEQ ID NO: 472 - DNA, 33 bp, Homo sapiens
cctataggaaacaccagtagtgacctacacagc

SEQ ID NO: 473 - DNA, 33 bp, Homo sapiens
ctcttctgtgggaagcaaaaacactgtgcacct

SEQ ID NO: 474 - DNA, 33 bp, Homo sapiens
ctcttctgtgggaagcgaaaacactgtgcacct

SEQ ID NO: 475 - DNA, 33 bp, Homo sapiens
ggaggatattaaagacggaactaccacaatttc

SEQ ID NO: 476 - DNA, 33 bp, Homo sapiens
ggaggatattaaagactgaactaccacaatttc

SEQ ID NO: 477 - DNA, 33 bp, Homo sapiens
acgtggatgtgaggtacgttaccaaataacttc

SEQ ID NO: 478 - DNA, 33 bp, Homo sapiens
acgtggatgtgaggtatgttaccaaataacttc

SEQ ID NO: 479 - DNA, 33 bp, Homo sapiens
agcacatcctattctccagtcccgcgggcggct

SEQ ID NO: 480 - DNA, 33 bp, Homo sapiens
agcacatcctattctctagtcccgcgggcggct

SEQ ID NO: 481 - DNA, 33 bp, Homo sapiens
cacttatgtcagagctgaagtcagtgaaccaaa

SEQ ID NO: 482 - DNA, 33 bp, Homo sapiens
cacttatgtcagagcttaagtcagtgaaccaaa

SEQ ID NO: 483 - DNA, 33 bp, Homo sapiens
aagcaccatatttgtccagaaaaaccacctctc

SEQ ID NO: 484 - DNA, 33 bp, Homo sapiens
aagcaccatatttgtctagaaaaaccacctctc

SEQ ID NO: 485 - DNA, 33 bp, Homo sapiens
ggcctgtcaaatgcaaatgcgtatgtgaatgtg

SEQ ID NO: 486 - DNA, 33 bp, Homo sapiens
ggcctgtcaaatgcaagtgcgtatgtgaatgtg

SEQ ID NO: 487 - DNA, 33 bp, Homo sapiens
caggatgtgccttaggcataatgtcaaatgggc

SEQ ID NO: 488 - DNA, 33 bp, Homo sapiens
caggatgtgccttaggtataatgtcaaatgggc

SEQ ID NO: 489 - DNA, 33 bp, Homo sapiens; identical to SEQ ID NO: 367

SEQ ID NO: 490 - DNA, 33 bp, Homo sapiens; identical to SEQ ID NO: 368

SEQ ID NO: 491 - DNA, 33 bp, Homo sapiens
acaagtgtaacagaaaaggaatgacagaaaaaa

SEQ ID NO: 492 - DNA, 33 bp, Homo sapiens
acaagtgtaacagaaatggaatgacagaaaaaa

SEQ ID NO: 493 - DNA, 33 bp, Homo sapiens
gatgtgggagaaggaaagttgtcctgggaatta

SEQ ID NO: 494 - DNA, 33 bp, Homo sapiens
gatgtgggagaaggaaggttgtcctgggaatta

SEQ ID NO: 495 - DNA, 33 bp, Homo sapiens
gttttaccaataatcaacataaacctaaaacta

SEQ ID NO: 496 - DNA, 33 bp, Homo sapiens
gttttaccaataatcaccataaacctaaaacta

SEQ ID NO: 497 - DNA, 33 bp, Homo sapiens
taaagaaaaacatggaagacttagcattcagag

SEQ ID NO: 498 - DNA, 33 bp, Homo sapiens
taaagaaaaacatggaggacttagcattcagag

SEQ ID NO: 499 - DNA, 33 bp, Homo sapiens
gatttagtgcccatgacgatgatgaattattag

SEQ ID NO: 500 - DNA, 33 bp, Homo sapiens
gatttagtgcccatgatgatgatgaattattag

SEQ ID NO: 501 - DNA, 33 bp, Homo sapiens
atctggcctgaaggaaatcactgtagtgaggaa

SEQ ID NO: 502 - DNA, 33 bp, Homo sapiens
atctggcctgaaggaattcactgtagtgaggaa

SEQ ID NO: 503 - DNA, 33 bp, Homo sapiens
ccagagtcagcgcccaatatattaaatagagag

SEQ ID NO: 504 - DNA, 33 bp, Homo sapiens
ccagagtcagcgcccattatattaaatagagag

SEQ ID NO: 505 - DNA, 33 bp, Homo sapiens
aagaaaaattcagtcacacaaaacaaagaaaat

SEQ ID NO: 506 - DNA, 33 bp, Homo sapiens
aagaaaaattcagtcatacaaaacaaagaaaat

SEQ ID NO: 507 - DNA, 33 bp, Homo sapiens
caggacttaggttcagcatgaggaaaatatctc

SEQ ID NO: 508 - DNA, 33 bp, Homo sapiens
caggacttaggttcagtatgaggaaaatatctc

SEQ ID NO: 509 - DNA, 33 bp, Homo sapiens
tcccgataaatcgtgtaaattatttgctcagcc

SEQ ID NO: 510 - DNA, 33 bp, Homo sapiens
tcccgataaatcgtgtcaattatttgctcagcc

SEQ ID NO: 511 - DNA, 33 bp, Homo sapiens
acaccataattaaaaaattggtcatttgttcca

SEQ ID NO: 512 - DNA, 33 bp, Homo sapiens
acaccataattaaaaagttggtcatttgttcca

SEQ ID NO: 513 - DNA, 33 bp, Homo sapiens
ctataaagtggctgcacggatgagggacattat

SEQ ID NO: 514 - DNA, 33 bp, Homo sapiens
ctataaagtggctgcatggatgagggacattat

SEQ ID NO: 515 - DNA, 33 bp, Homo sapiens
gaaaatgttatcaggaaagcaggattaaataag

SEQ ID NO: 516 - DNA, 33 bp, Homo sapiens
gaaaatgttatcaggagagcaggattaaataag

SEQ ID NO: 517 - DNA, 33 bp, Homo sapiens
gtgctggagtaataggccaaatgtatcatggcc

SEQ ID NO: 518 - DNA, 33 bp, Homo sapiens
gtgctggagtaataggtcaaatgtatcatggcc

SEQ ID NO: 519 - DNA, 33 bp, Homo sapiens
agctctaatatttataatacccaccacggttgt

SEQ ID NO: 520 - DNA, 33 bp, Homo sapiens
agctctaatatttatagtacccaccacggttgt

SEQ ID NO: 521 - DNA, 33 bp, Homo sapiens
aggtcttcggggtttccatctgagggtgtatga

SEQ ID NO: 522 - DNA, 33 bp, Homo sapiens
aggtcttcggggtttctatctgagggtgtatga

SEQ ID NO: 523 - DNA, 33 bp, Homo sapiens
gaccttttctgatcaaaccctgtgtatggctga

SEQ ID NO: 524 - DNA, 33 bp, Homo sapiens
gaccttttctgatcaagccctgtgtatggctga

SEQ ID NO: 525 - DNA, 33 bp, Homo sapiens
tcctccttctgtttaacacatttttccatctca

SEQ ID NO: 526 - DNA, 33 bp, Homo sapiens
tcctccttctgtttaatacatttttccatctca

SEQ ID NO: 527 - DNA, 33 bp, Homo sapiens
aaaaagaaaaagaaaactgagcttatagtacag

SEQ ID NO: 528 - DNA, 33 bp, Homo sapiens
aaaaagaaaaagaaaattgagcttatagtacag

SEQ ID NO: 529 - DNA, 33 bp, Homo sapiens
ctttcacttcacactgaaccctacaaatgacgt

SEQ ID NO: 530 - DNA, 33 bp, Homo sapiens
ctttcacttcacactggaccctacaaatgacgt

SEQ ID NO: 531 - DNA, 33 bp, Homo sapiens
aaaaagaattcacccaagcttacatagctagta

SEQ ID NO: 532 - DNA, 33 bp, Homo sapiens
aaaaagaattcacccacgcttacatagctagta

SEQ ID NO: 533 - DNA, 33 bp, Homo sapiens
attgcagattacataactgggattaagtgaggt

SEQ ID NO: 534 - DNA, 33 bp, Homo sapiens
attgcagattacataattgggattaagtgaggt

SEQ ID NO: 535 - DNA, 33 bp, Homo sapiens
ggagtccagtcgttagcaggagttctggaggag

SEQ ID NO: 536 - DNA, 33 bp, Homo sapiens
ggagtccagtcgttagtaggagttctggaggag

SEQ ID NO: 537 - DNA, 33 bp, Homo sapiens
aaccaccatcatctcactatctcttgtcttgta

SEQ ID NO: 538 - DNA, 33 bp, Homo sapiens
aaccaccatcatctcagtatctcttgtcttgta

SEQ ID NO: 539 - DNA, 33 bp, Homo sapiens
attagtatacagtggcaattaacagaagcagaa

SEQ ID NO: 540 - DNA, 33 bp, Homo sapiens
attagtatacagtggcgattaacagaagcagaa

SEQ ID NO: 541 - DNA, 33 bp, Homo sapiens
gaaagtgccaggggaactgcaacttggcaatgg

SEQ ID NO: 542 - DNA, 33 bp, Homo sapiens
gaaagtgccaggggaagtgcaacttggcaatgg

SEQ ID NO: 543 - DNA, 33 bp, Homo sapiens
gcagagatagcagaaagcaagaaaaaggttgga

SEQ ID NO: 544 - DNA, 33 bp, Homo sapiens
gcagagatagcagaaatcaagaaaaaggttgga

SEQ ID NO: 545 - DNA, 33 bp, Homo sapiens
gaagctattagttttacgccacttcatagagag

SEQ ID NO: 546 - DNA, 33 bp, Homo sapiens
gaagctattagttttatgccacttcatagagag

SEQ ID NO: 547 - DNA, 33 bp, Homo sapiens
ttgtagctaggatcccagatgtctgcagtgcaa

SEQ ID NO: 548 - DNA, 33 bp, Homo sapiens
ttgtagctaggatcccggatgtctgcagtgcaa

SEQ ID NO: 549 - DNA, 33 bp, Homo sapiens
gaaacataagctcttacaggtctaaagcctgcc

SEQ ID NO: 550 - DNA, 33 bp, Homo sapiens
gaaacataagctcttataggtctaaagcctgcc

SEQ ID NO: 551 - DNA, 33 bp, Homo sapiens
caactgtagtggataacgtcaagttgtttttct

SEQ ID NO: 552 - DNA, 33 bp, Homo sapiens
caactgtagtggataatgtcaagttgtttttct

SEQ ID NO: 553 - DNA, 33 bp, Homo sapiens
cactggcctggggtaggagctaaagccatatgc

SEQ ID NO: 554 - DNA, 33 bp, Homo sapiens
cactggcctggggtagtagctaaagccatatgc

SEQ ID NO: 555 - DNA, 33 bp, Homo sapiens
attctgtttttagttcaagggccattgtttatt

SEQ ID NO: 556 - DNA, 33 bp, Homo sapiens
attctgtttttagttcgagggccattgtttatt

SEQ ID NO: 557 - DNA, 33 bp, Homo sapiens
aatgtagagcctgagaacctaaggaagtatctg

SEQ ID NO: 558 - DNA, 33 bp, Homo sapiens
aatgtagagcctgagagcctaaggaagtatctg

SEQ ID NO: 559 - DNA, 33 bp, Homo sapiens
tgctgcagttatgtagaaagcaggttgtcatgg

SEQ ID NO: 560 - DNA, 33 bp, Homo sapiens
tgctgcagttatgtagcaagcaggttgtcatgg

SEQ ID NO: 561 - DNA, 33 bp, Homo sapiens
tgagagaactccaagaaggatgtcgaaactggg

SEQ ID NO: 562 - DNA, 33 bp, Homo sapiens
tgagagaactccaagagggatgtcgaaactggg

SEQ ID NO: 563 - DNA, 33 bp, Homo sapiens
gcacagagtagaggcacggtaacgatgtgttgt

SEQ ID NO: 564 - DNA, 33 bp, Homo sapiens
gcacagagtagaggcatggtaacgatgtgttgt

SEQ ID NO: 565 - DNA, 33 bp, Homo sapiens
agtgttctggtctggacgaaatgagagaactcc

SEQ ID NO: 566 - DNA, 33 bp, Homo sapiens
agtgttctggtctggatgaaatgagagaactcc

SEQ ID NO: 567 - DNA, 33 bp, Homo sapiens
ttgtcataataactacaatttatagagtgccta

SEQ ID NO: 568 - DNA, 33 bp, Homo sapiens
ttgtcataataactaccatttatagagtgccta

SEQ ID NO: 569 - DNA, 33 bp, Homo sapiens
ccctgccctaatcaccaagcagacaggcaacat

SEQ ID NO: 570 - DNA, 33 bp, Homo sapiens
ccctgccctaatcaccgagcagacaggcaacat

SEQ ID NO: 571 - DNA, 33 bp, Homo sapiens
ggggccctgttggttcactgatcatgagaacct

SEQ ID NO: 572 - DNA, 33 bp, Homo sapiens
ggggccctgttggttcgctgatcatgagaacct

SEQ ID NO: 573 - DNA, 33 bp, Homo sapiens
cccacagggggtggaacgacaagcaagtaacca

SEQ ID NO: 574 - DNA, 33 bp, Homo sapiens
cccacagggggtggaaggacaagcaagtaacca

SEQ ID NO: 575 - DNA, 33 bp, Homo sapiens; identical to SEQ ID NO: 375

SEQ ID NO: 576 - DNA, 33 bp, Homo sapiens; identical to SEQ ID NO: 376

SEQ ID NO: 577 - DNA, 33 bp, Homo sapiens
cagaggggaaagcacaatagacaaggtcagcct

SEQ ID NO: 578 - DNA, 33 bp, Homo sapiens
cagaggggaaagcacagtagacaaggtcagcct

SEQ ID NO: 579 - DNA, 33 bp, Homo sapiens
aatcctcatccccagcaaataattattgagtcc

SEQ ID NO: 580 - DNA, 33 bp, Homo sapiens
aatcctcatccccagcgaataattattgagtcc

SEQ ID NO: 581 - DNA, 33 bp, Homo sapiens
tcatttattaaaacacagcatgattagaccact

SEQ ID NO: 582 - DNA, 33 bp, Homo sapiens
tcatttattaaaacacggcatgattagaccact

SEQ ID NO: 583 - DNA, 33 bp, Homo sapiens
cagtagttagaatagaaataagttgtggcttca

SEQ ID NO: 584 - DNA, 33 bp, Homo sapiens
cagtagttagaatagagataagttgtggcttca

SEQ ID NO: 585 - DNA, 33 bp, Homo sapiens
atcagatggtatttgtcaacttactgctctgaa

SEQ ID NO: 586 - DNA, 33 bp, Homo sapiens
atcagatggtatttgttaacttactgctctgaa

SEQ ID NO: 587 - DNA, 33 bp, Homo sapiens
aagttcttattacgcaacagtcactgtgctaag

SEQ ID NO: 588 - DNA, 33 bp, Homo sapiens
aagttcttattacgcatcagtcactgtgctaag

SEQ ID NO: 589 - DNA, 33 bp, Homo sapiens
aactacctctactaaccaccatctaagcatgga

SEQ ID NO: 590 - DNA, 33 bp, Homo sapiens
aactacctctactaactaccatctaagcatgga

SEQ ID NO: 591 - DNA, 33 bp, Homo sapiens
agcatgggcctgaatcaaaaatgactggcattc

SEQ ID NO: 592 - DNA, 33 bp, Homo sapiens
agcatgggcctgaatccaaaatgactggcattc

SEQ ID NO: 593 - DNA, 33 bp, Homo sapiens
ggatgttctgtttagaagaagttaagagctgag

SEQ ID NO: 594 - DNA, 33 bp, Homo sapiens
ggatgttctgtttagaggaagttaagagctgag

SEQ ID NO: 595 - DNA, 33 bp, Homo sapiens
atgcacagcacggttgcagatttttaacattag

SEQ ID NO: 596 - DNA, 33 bp, Homo sapiens
atgcacagcacggttgtagatttttaacattag

SEQ ID NO: 597 - DNA, 33 bp, Homo sapiens
tgattaggaaatgtcaaggatacgtctgtaaaa

SEQ ID NO: 598 - DNA, 33 bp, Homo sapiens
tgattaggaaatgtcatggatacgtctgtaaaa

SEQ ID NO: 599 - DNA, 33 bp, Homo sapiens
ttgtgaaataactgtcccagactcttcaaaaat

SEQ ID NO: 600 - DNA, 33 bp, Homo sapiens
ttgtgaaataactgtctcagactcttcaaaaat

SEQ ID NO: 601 - DNA, 33 bp, Homo sapiens
cttacattctctaatacgagttgaggtgcctag

SEQ ID NO: 602 - DNA, 33 bp, Homo sapiens
cttacattctctaatatgagttgaggtgcctag

SEQ ID NO: 603 - DNA, 33 bp, Homo sapiens
ctgtttacttgctccaatctggccacactggcc

SEQ ID NO: 604 - DNA, 33 bp, Homo sapiens
ctgtttacttgctccagtctggccacactggcc

SEQ ID NO: 605 - DNA, 33 bp, Homo sapiens
taagcaaagccaggataatcattcttattccat

SEQ ID NO: 606 - DNA, 33 bp, Homo sapiens
taagcaaagccaggatcatcattcttattccat

SEQ ID NO: 607 - DNA, 33 bp, Homo sapiens
ggtcatcgaccagtacagcatcccacaggtaag

SEQ ID NO: 608 - DNA, 33 bp, Homo sapiens
ggtcatcgaccagtacggcatcccacaggtaag

SEQ ID NO: 609 - DNA, 33 bp, Homo sapiens
gggatgaatgagttcaaagtcttgcaaaaggga

SEQ ID NO: 610 - DNA, 33 bp, Homo sapiens
gggatgaatgagttcagagtcttgcaaaaggga

SEQ ID NO: 611 - DNA, 33 bp, Homo sapiens
acaaaaaactaaaatacagtccatttggtccag

SEQ ID NO: 612 - DNA, 33 bp, Homo sapiens
acaaaaaactaaaatagagtccatttggtccag

SEQ ID NO: 613 - DNA, 33 bp, Homo sapiens
cccatcctttaaacagaaacagtttgtgaagat

SEQ ID NO: 614 - DNA, 33 bp, Homo sapiens
cccatcctttaaacaggaacagtttgtgaagat

SEQ ID NO: 615 - DNA, 33 bp, Homo sapiens
aacctttgtatgagcccattagctgcctcagct

SEQ ID NO: 616 - DNA, 33 bp, Homo sapiens
aacctttgtatgagcctattagctgcctcagct

SEQ ID NO: 617 - DNA, 33 bp, Homo sapiens
tatatgtgaaatacccaaacttggaatgcattt

SEQ ID NO: 618 - DNA, 33 bp, Homo sapiens
tatatgtgaaatacccgaacttggaatgcattt

SEQ ID NO: 619 - DNA, 33 bp, Homo sapiens
tatttctctactgtgccgatactgtgtccacac

SEQ ID NO: 620 - DNA, 33 bp, Homo sapiens
tatttctctactgtgctgatactgtgtccacac

SEQ ID NO: 621 - DNA, 33 bp, Homo sapiens
aacatccaacaacaaagatccaattaaataaag

SEQ ID NO: 622 - DNA, 33 bp, Homo sapiens
aacatccaacaacaaatatccaattaaataaag

SEQ ID NO: 623 - DNA, 33 bp, Homo sapiens
acctctcacctcagcaaattcttcagtaataat

SEQ ID NO: 624 - DNA, 33 bp, Homo sapiens
acctctcacctcagcagattcttcagtaataat

SEQ ID NO: 625 - DNA, 33 bp, Homo sapiens
aaacttcacatcattaaaaacattcggctgagt

SEQ ID NO: 626 - DNA, 33 bp, Homo sapiens
aaacttcacatcattagaaacattcggctgagt

SEQ ID NO: 627 - DNA, 33 bp, Homo sapiens
agctgttctgtacttaagtcacacccagaattg

SEQ ID NO: 628 - DNA, 33 bp, Homo sapiens
agctgttctgtacttacgtcacacccagaattg

SEQ ID NO: 629 - DNA, 33 bp, Homo sapiens
atggacagtcttacaactctgggtgtgacttaa

SEQ ID NO: 630 - DNA, 33 bp, Homo sapiens
atggacagtcttacaattctgggtgtgacttaa

SEQ ID NO: 631 - DNA, 33 bp, Homo sapiens
caaggacgaaagatacacaaccaagggaatcac

SEQ ID NO: 632 - DNA, 33 bp, Homo sapiens
caaggacgaaagatacgcaaccaagggaatcac

SEQ ID NO: 633 - DNA, 33 bp, Homo sapiens
ttcagagtgattgtaaaaggcctgcatgatgca

SEQ ID NO: 634 - DNA, 33 bp, Homo sapiens
ttcagagtgattgtaagaggcctgcatgatgca

SEQ ID NO: 635 - DNA, 33 bp, Homo sapiens
aaacaaaaataagcggcccccatcttaaatctt

SEQ ID NO: 636 - DNA, 33 bp, Homo sapiens
aaacaaaaataagcgggccccatcttaaatctt

SEQ ID NO: 637 - DNA, 33 bp, Homo sapiens
taaggatgtcgtgtaaacatatcattaatgtag

SEQ ID NO: 638 - DNA, 33 bp, Homo sapiens
taaggatgtcgtgtaagcatatcattaatgtag

SEQ ID NO: 639 - DNA, 33 bp, Homo sapiens
ggaatgaaataatggacggtatttgcagcaacc

SEQ ID NO: 640 - DNA, 33 bp, Homo sapiens
ggaatgaaataatggatggtatttgcagcaacc

SEQ ID NO: 641 - DNA, 33 bp, Homo sapiens
gtgacttaacgtagtacgagaactgttgagctt

SEQ ID NO: 642 - DNA, 33 bp, Homo sapiens
gtgacttaacgtagtatgagaactgttgagctt

SEQ ID NO: 643 - DNA, 33 bp, Homo sapiens
tcctgtctcaaagaggcaatatagtgtgaattg

SEQ ID NO: 644 - DNA, 33 bp, Homo sapiens
tcctgtctcaaagaggtaatatagtgtgaattg

SEQ ID NO: 645 - DNA, 33 bp, Homo sapiens
cactgccttagaacataagtgcttccatacaga

SEQ ID NO: 646 - DNA, 33 bp, Homo sapiens
cactgccttagaacatgagtgcttccatacaga

SEQ ID NO: 647 - DNA, 33 bp, Homo sapiens
gcctaacaacagagtacacatgagccaaaatct

SEQ ID NO: 648 - DNA, 33 bp, Homo sapiens
gcctaacaacagagtatacatgagccaaaatct

SEQ ID NO: 649 - DNA, 33 bp, Homo sapiens
tgtgcgcagaagcagcagattaacggaagaaca

SEQ ID NO: 650 - DNA, 33 bp, Homo sapiens
tgtgcgcagaagcagccgattaacggaagaaca

SEQ ID NO: 651 - DNA, 33 bp, Homo sapiens
gcagtggctacattccagaaaacagccgcagaa

SEQ ID NO: 652 - DNA, 33 bp, Homo sapiens
gcagtggctacattccggaaaacagccgcagaa

SEQ ID NO: 653 - DNA, 33 bp, Homo sapiens
aaataaattgaattggccagtctagcttgcaaa

SEQ ID NO: 654 - DNA, 33 bp, Homo sapiens
aaataaattgaattggtcagtctagcttgcaaa

SEQ ID NO: 655 - DNA, 33 bp, Homo sapiens
aaaaagtgagagtggcaaaaagtatacttgata

SEQ ID NO: 656 - DNA, 33 bp, Homo sapiens
aaaaagtgagagtggcgaaaagtatacttgata

SEQ ID NO: 657 - DNA, 33 bp, Homo sapiens
aaatactcaactccgaatagctctagtgtttca

SEQ ID NO: 658 - DNA, 33 bp, Homo sapiens
aaatactcaactccgactagctctagtgtttca

SEQ ID NO: 659 - DNA, 33 bp, Homo sapiens
agtctacaaacaacacagagtaataacattata

SEQ ID NO: 660 - DNA, 33 bp, Homo sapiens
agtctacaaacaacacggagtaataacattata

SEQ ID NO: 661 - DNA, 33 bp, Homo sapiens
atgcaaaatgcacatagtagtaattagtcaaac

SEQ ID NO: 662 - DNA, 33 bp, Homo sapiens
atgcaaaatgcacatattagtaattagtcaaac

SEQ ID NO: 663 - DNA, 33 bp, Homo sapiens
aggcacttagcatacaacaattaagatggaagt

SEQ ID NO: 664 - DNA, 33 bp, Homo sapiens
aggcacttagcatacagcaattaagatggaagt

SEQ ID NO: 665 - DNA, 33 bp, Homo sapiens
agataaccgaagctaactgaccacatcctactt

SEQ ID NO: 666 - DNA, 33 bp, Homo sapiens
agataaccgaagctaattgaccacatcctactt

SEQ ID NO: 667 - DNA, 33 bp, Homo sapiens
atatggtaggaccttacgtgactgaagctatct

SEQ ID NO: 668 - DNA, 33 bp, Homo sapiens
atatggtaggaccttatgtgactgaagctatct

SEQ ID NO: 669 - DNA, 33 bp, Homo sapiens
attctgagatctttccactatttcaggctctag

SEQ ID NO: 670 - DNA, 33 bp, Homo sapiens
attctgagatctttccgctatttcaggctctag

SEQ ID NO: 671 - DNA, 33 bp, Homo sapiens
gcctgcagctaggctacagagagttctggaaga

SEQ ID NO: 672 - DNA, 33 bp, Homo sapiens
gcctgcagctaggctagagagagttctggaaga

SEQ ID NO: 673 - DNA, 33 bp, Homo sapiens
gtcactccctgtccaaatcaaacctggattctg

SEQ ID NO: 674 - DNA, 33 bp, Homo sapiens
gtcactccctgtccaagtcaaacctggattctg

SEQ ID NO: 675 - DNA, 33 bp, Homo sapiens
catggaggcaaactacagtagctgattaccatt

SEQ ID NO: 676 - DNA, 33 bp, Homo sapiens
catggaggcaaactacggtagctgattaccatt

SEQ ID NO: 677 - DNA, 33 bp, Homo sapiens
gaaaatatttgaggataaatgtctaaatgcaag

SEQ ID NO: 678 - DNA, 33 bp, Homo sapiens
gaaaatatttgaggatgaatgtctaaatgcaag

SEQ ID NO: 679 - DNA, 33 bp, Homo sapiens
gaagcaacttagtttcccaatgaaagctgtaac

SEQ ID NO: 680 - DNA, 33 bp, Homo sapiens
gaagcaacttagtttctcaatgaaagctgtaac

SEQ ID NO: 681 - DNA, 33 bp, Homo sapiens
ttccacaaattgttaaacaaactttctgtagaa

SEQ ID NO: 682 - DNA, 33 bp, Homo sapiens
ttccacaaattgttaatcaaactttctgtagaa

SEQ ID NO: 683 - DNA, 33 bp, Homo sapiens
tccctccacagccacacgttgcttttagcttgc

SEQ ID NO: 684 - DNA, 33 bp, Homo sapiens
tccctccacagccacatgttgcttttagcttgc

SEQ ID NO: 685 - DNA, 33 bp, Homo sapiens
tcaaaggaattttgcacgtatttacatctctct

SEQ ID NO: 686 - DNA, 33 bp, Homo sapiens
tcaaaggaattttgcaggtatttacatctctct

SEQ ID NO: 687 - DNA, 33 bp, Homo sapiens
cctgatcctctgtaaacccttaatgaatgtaat

SEQ ID NO: 688 - DNA, 33 bp, Homo sapiens
cctgatcctctgtaaagccttaatgaatgtaat

SEQ ID NO: 689 - DNA, 33 bp, Homo sapiens
tgcatttgttaaaggccgatgtgcttcttaaca

SEQ ID NO: 690 - DNA, 33 bp, Homo sapiens
tgcatttgttaaaggctgatgtgcttcttaaca

SEQ ID NO: 691 - DNA, 33 bp, Homo sapiens
tctccttcctccaaacagctctaatactttcat

SEQ ID NO: 692 - DNA, 33 bp, Homo sapiens
tctccttcctccaaacggctctaatactttcat

SEQ ID NO: 693 - DNA, 33 bp, Homo sapiens
actgtcttttcaagcaagttcaaacaagcctga

SEQ ID NO: 694 - DNA, 33 bp, Homo sapiens
actgtcttttcaagcaggttcaaacaagcctga

SEQ ID NO: 695 - DNA, 33 bp, Homo sapiens
atcatccaccaagcagacatctttcctgagaag

SEQ ID NO: 696 - DNA, 33 bp, Homo sapiens
atcatccaccaagcaggcatctttcctgagaag

SEQ ID NO: 697 - DNA, 33 bp, Homo sapiens
acacaccaagctcatccaccccagtatcccact

SEQ ID NO: 698 - DNA, 33 bp, Homo sapiens
acacaccaagctcatcgaccccagtatcccact

SEQ ID NO: 699 - DNA, 33 bp, Homo sapiens
tttcattctgagatgcaaaatcattgagcagaa

SEQ ID NO: 700 - DNA, 33 bp, Homo sapiens
tttcattctgagatgcgaaatcattgagcagaa

SEQ ID NO: 701 - DNA, 33 bp, Homo sapiens
cattcctgaatgtttgaaaatagaagaggcttc

SEQ ID NO: 702 - DNA, 33 bp, Homo sapiens
cattcctgaatgtttgcaaatagaagaggcttc

SEQ ID NO: 703 - DNA, 33 bp, Homo sapiens
aaggcgctagagagtacgacttgtggctgttag

SEQ ID NO: 704 - DNA, 33 bp, Homo sapiens
aaggcgctagagagtatgacttgtggctgttag

SEQ ID NO: 705 - DNA, 33 bp, Homo sapiens
ccacagtgaggctaaacaccctggatctcttgg

SEQ ID NO: 706 - DNA, 33 bp, Homo sapiens
ccacagtgaggctaaataccctggatctcttgg

SEQ ID NO: 707 - DNA, 33 bp, Homo sapiens
tatttgtgggttcatacatgatttttcagatgt

SEQ ID NO: 708 - DNA, 33 bp, Homo sapiens
tatttgtgggttcatagatgatttttcagatgt

SEQ ID NO: 709 - DNA, 33 bp, Homo sapiens
gtcaggatgcatttcaaaatcatgagaaagaag

SEQ ID NO: 710 - DNA, 33 bp, Homo sapiens
gtcaggatgcatttcagaatcatgagaaagaag

SEQ ID NO: 711 - DNA, 33 bp, Homo sapiens
aaagggattgtgaaaactgttaatgagaaagca

SEQ ID NO: 712 - DNA, 33 bp, Homo sapiens
aaagggattgtgaaaattgttaatgagaaagca

SEQ ID NO: 713 - DNA, 33 bp, Homo sapiens
tagaaccagagcagaacgtagcctcaggaagaa

SEQ ID NO: 714 - DNA, 33 bp, Homo sapiens
tagaaccagagcagaatgtagcctcaggaagaa

SEQ ID NO: 715 - DNA, 33 bp, Homo sapiens
ctctctcattttctcaaggaactcttctgcatc

SEQ ID NO: 716 - DNA, 33 bp, Homo sapiens
ctctctcattttctcagggaactcttctgcatc

SEQ ID NO: 717 - DNA, 33 bp, Homo sapiens
tatgtaaggtgggctaaagaaatgtttaaaaag

SEQ ID NO: 718 - DNA, 33 bp, Homo sapiens
tatgtaaggtgggctagagaaatgtttaaaaag

SEQ ID NO: 719 - DNA, 33 bp, Homo sapiens
ggttctcagatgaagaagtcataagctacctgg

SEQ ID NO: 720 - DNA, 33 bp, Homo sapiens
ggttctcagatgaagaggtcataagctacctgg

SEQ ID NO: 721 - DNA, 33 bp, Homo sapiens
ttactttaaatgtacacaacaatctggctaagt

SEQ ID NO: 722 - DNA, 33 bp, Homo sapiens
ttactttaaatgtacagaacaatctggctaagt

SEQ ID NO: 723 - DNA, 33 bp, Homo sapiens
gtttctgctctttctaacgcgtgatagcggcgc

SEQ ID NO: 724 - DNA, 33 bp, Homo sapiens
gtttctgctctttctaccgcgtgatagcggcgc

SEQ ID NO: 725 - DNA, 33 bp, Homo sapiens
ggaatacagtatcatacagacatggttttttat

SEQ ID NO: 726 - DNA, 33 bp, Homo sapiens
ggaatacagtatcatagagacatggttttttat

SEQ ID NO: 727 - DNA, 33 bp, Homo sapiens
tctttgcaattttgaaccatctaattctaccta

SEQ ID NO: 728 - DNA, 33 bp, Homo sapiens
tctttgcaattttgaatcatctaattctaccta

SEQ ID NO: 729 - DNA, 33 bp, Homo sapiens
taccatttcaaattcactattcaagccatatat

SEQ ID NO: 730 - DNA, 33 bp, Homo sapiens
taccatttcaaattcagtattcaagccatatat

SEQ ID NO: 731 - DNA, 33 bp, Homo sapiens
tatagctctttggtaacaacagaaaggtcattt

SEQ ID NO: 732 - DNA, 33 bp, Homo sapiens
tatagctctttggtaataacagaaaggtcattt

SEQ ID NO: 733 - DNA, 33 bp, Homo sapiens
aaaggaactattcttcactacacgagcactaaa

SEQ ID NO: 734 - DNA, 33 bp, Homo sapiens
aaaggaactattcttcgctacacgagcactaaa

SEQ ID NO: 735 - DNA, 33 bp, Homo sapiens
ggaaagcaacgaaagcaacggcagttggacttc

SEQ ID NO: 736 - DNA, 33 bp, Homo sapiens
ggaaagcaacgaaagcgacggcagttggacttc

SEQ ID NO: 737 - DNA, 33 bp, Homo sapiens
aatctcagatcatttaactacatcaaacatctc

SEQ ID NO: 738 - DNA, 33 bp, Homo sapiens
aatctcagatcatttacctacatcaaacatctc

SEQ ID NO: 739 - DNA, 33 bp, Homo sapiens
caattgatagagaaaaacacagagctctccaca

SEQ ID NO: 740 - DNA, 33 bp, Homo sapiens
caattgatagagaaaatcacagagctctccaca

SEQ ID NO: 741 - DNA, 33 bp, Homo sapiens
acactgattctgcagagagttggcaatttcttc

SEQ ID NO: 742 - DNA, 33 bp, Homo sapiens
acactgattctgcagatagttggcaatttcttc

SEQ ID NO: 743 - DNA, 33 bp, Homo sapiens
aggcaaaagagatagcaagagcggtggggggtt

SEQ ID NO: 744 - DNA, 33 bp, Homo sapiens
aggcaaaagagatagcgagagcggtggggggtt

SEQ ID NO: 745 - DNA, 33 bp, Homo sapiens
caaagaacgcttaatggaaggaagaagtattag

SEQ ID NO: 746 - DNA, 33 bp, Homo sapiens
caaagaacgcttaatgtaaggaagaagtattag

SEQ ID NO: 747 - DNA, 33 bp, Homo sapiens
ttgacatatgcacagaggatctcttttgattct

SEQ ID NO: 748 - DNA, 33 bp, Homo sapiens
ttgacatatgcacagatgatctcttttgattct

SEQ ID NO: 749 - DNA, 33 bp, Homo sapiens
ttgctgagtaagcaaacatagcatttttcttca

SEQ ID NO: 750 - DNA, 33 bp, Homo sapiens
ttgctgagtaagcaaatatagcatttttcttca

SEQ ID NO: 751 - DNA, 33 bp, Homo sapiens
cagagtgttaaagtaactggcctaaggtgtaca

SEQ ID NO: 752 - DNA, 33 bp, Homo sapiens
cagagtgttaaagtaagtggcctaaggtgtaca

SEQ ID NO: 753 - DNA, 33 bp, Homo sapiens
gccacacccttagccaatcttgggctggatttc

SEQ ID NO: 754 - DNA, 33 bp, Homo sapiens
gccacacccttagccagtcttgggctggatttc

SEQ ID NO: 755 - DNA, 33 bp, Homo sapiens
atgagtctttcccacaatgcctaaaaagattct

SEQ ID NO: 756 - DNA, 33 bp, Homo sapiens
atgagtctttcccacagtgcctaaaaagattct

SEQ ID NO: 757 - DNA, 33 bp, Homo sapiens
aattcttttggcaatacatctagaaatggagtt

SEQ ID NO: 758 - DNA, 33 bp, Homo sapiens
aattcttttggcaatatatctagaaatggagtt

SEQ ID NO: 759 - DNA, 33 bp, Homo sapiens
tgcagcctctgcaaccgacttccgtgtgctttc

SEQ ID NO: 760 - DNA, 33 bp, Homo sapiens
tgcagcctctgcaacctacttccgtgtgctttc

SEQ ID NO: 761 - DNA, 33 bp, Homo sapiens
atctatattgaatccaacatcttaagttgcctg

SEQ ID NO: 762 - DNA, 33 bp, Homo sapiens
atctatattgaatccagcatcttaagttgcctg

SEQ ID NO: 763 - DNA, 33 bp, Homo sapiens
catttccaagaagcaacgcaaagtaagttttcc

SEQ ID NO: 764 - DNA, 33 bp, Homo sapiens
catttccaagaagcaatgcaaagtaagttttcc

SEQ ID NO: 765 - DNA, 33 bp, Homo sapiens
aggagagggttagaaaatatggaatgaaaatag

SEQ ID NO: 766 - DNA, 33 bp, Homo sapiens
aggagagggttagaaagtatggaatgaaaatag

SEQ ID NO: 767 - DNA, 33 bp, Homo sapiens
gaaaacaaaaagattacgcaagaggtcaagaag

SEQ ID NO: 768 - DNA, 33 bp, Homo sapiens
gaaaacaaaaagattatgcaagaggtcaagaag

SEQ ID NO: 769 - DNA, 33 bp, Homo sapiens
tggcatcaaaggtgggaacctgtctttattatc

SEQ ID NO: 770 - DNA, 33 bp, Homo sapiens
tggcatcaaaggtggggacctgtctttattatc

SEQ ID NO: 771 - DNA, 33 bp, Homo sapiens
ctgtggtccagctctacacgccaaaaggagaga

SEQ ID NO: 772 - DNA, 33 bp, Homo sapiens
ctgtggtccagctctagacgccaaaaggagaga

SEQ ID NO: 773 - DNA, 33 bp, Homo sapiens
taaagtatcctgtggaccaaggctaaggctaag

SEQ ID NO: 774 - DNA, 33 bp, Homo sapiens
taaagtatcctgtggatcaaggctaaggctaag

SEQ ID NO: 775 - DNA, 33 bp, Homo sapiens
tccctgatccattgaacctaaaagtctgagact

SEQ ID NO: 776 - DNA, 33 bp, Homo sapiens
tccctgatccattgaatctaaaagtctgagact

SEQ ID NO: 777 - DNA, 33 bp, Homo sapiens
aacttaacagaactagaaaagagatcgaagaac

SEQ ID NO: 778 - DNA, 33 bp, Homo sapiens
aacttaacagaactagtaaagagatcgaagaac

SEQ ID NO: 779 - DNA, 33 bp, Homo sapiens
ttgtgtagagtaactccatgcgtaaacagtttc

SEQ ID NO: 780 - DNA, 33 bp, Homo sapiens
ttgtgtagagtaactctatgcgtaaacagtttc

SEQ ID NO: 781 - DNA, 33 bp, Homo sapiens
agaagagtacaccatgccagtagtcatcaaaaa

SEQ ID NO: 782 - DNA, 33 bp, Homo sapiens
agaagagtacaccatgtcagtagtcatcaaaaa

SEQ ID NO: 783 - DNA, 33 bp, Homo sapiens
aagtctctgggtcacactagcctttaccatctt

SEQ ID NO: 784 - DNA, 33 bp, Homo sapiens
aagtctctgggtcacattagcctttaccatctt

SEQ ID NO: 785 - DNA, 33 bp, Homo sapiens
tggctcatactcaccaagctgtgtccagagtct

SEQ ID NO: 786 - DNA, 33 bp, Homo sapiens
tggctcatactcaccaggctgtgtccagagtct

SEQ ID NO: 787 - DNA, 33 bp, Homo sapiens
cacacaacaatttagacggatctcaaggggatt

SEQ ID NO: 788 - DNA, 33 bp, Homo sapiens
cacacaacaatttagatggatctcaaggggatt

SEQ ID NO: 789 - DNA, 33 bp, Homo sapiens; identical to SEQ ID NO: 341

SEQ ID NO: 790 - DNA, 33 bp, Homo sapiens; identical to SEQ ID NO: 342

SEQ ID NO: 791 - DNA, 33 bp, Homo sapiens
actgaatttatgctgacgtgactataatcttca

SEQ ID NO: 792 - DNA, 33 bp, Homo sapiens
actgaatttatgctgatgtgactataatcttca

SEQ ID NO: 793 - DNA, 33 bp, Homo sapiens; identical to SEQ ID NO: 343

SEQ ID NO: 794 - DNA, 33 bp, Homo sapiens; identical to SEQ ID NO: 344

SEQ ID NO: 795 - DNA, 33 bp, Homo sapiens; identical to SEQ ID NO: 347

SEQ ID NO: 796 - DNA, 33 bp, Homo sapiens; identical to SEQ ID NO: 348

SEQ ID NO: 797 - DNA, 33 bp, Homo sapiens; identical to SEQ ID NO: 351

SEQ ID NO: 798 - DNA, 33 bp, Homo sapiens; identical to SEQ ID NO: 352

SEQ ID NO: 799 - DNA, 33 bp, Homo sapiens
tacatcaggtaatctacactgagctaatgtagc

SEQ ID NO: 800 - DNA, 33 bp, Homo sapiens
tacatcaggtaatctatactgagctaatgtagc

SEQ ID NO: 801 - DNA, 33 bp, Homo sapiens; identical to SEQ ID NO: 275

SEQ ID NO: 802 - DNA, 33 bp, Homo sapiens; identical to SEQ ID NO: 276

SEQ ID NO: 803 - DNA, 33 bp, Homo sapiens; identical to SEQ ID NO: 215

SEQ ID NO: 804 - DNA, 33 bp, Homo sapiens; identical to SEQ ID NO: 216

SEQ ID NO: 805 - DNA, 33 bp, Homo sapiens; identical to SEQ ID NO: 295

SEQ ID NO: 806 - DNA, 33 bp, Homo sapiens; identical to SEQ ID NO: 296

SEQ ID NO: 807 - DNA, 33 bp, Homo sapiens
aaatggcccaggctgcaaaaaaggggagcagca

SEQ ID NO: 808 - DNA, 33 bp, Homo sapiens
aaatggcccaggctgccaaaaaggggagcagca

SEQ ID NO: 809 - DNA, 33 bp, Homo sapiens
agtaaagagtggttgacagtggagctcagaagg

SEQ ID NO: 810 - DNA, 33 bp, Homo sapiens
agtaaagagtggttgatagtggagctcagaagg

SEQ ID NO: 811 - DNA, 33 bp, Homo sapiens
ctccatcactgggtgcaacactccttacagagc

SEQ ID NO: 812 - DNA, 33 bp, Homo sapiens
ctccatcactgggtgcgacactccttacagagc

SEQ ID NO: 813 - DNA, 33 bp, Homo sapiens
agttttattacttacaaatcagtagcaagggac

SEQ ID NO: 814 - DNA, 33 bp, Homo sapiens
agttttattacttacagatcagtagcaagggac

SEQ ID NO: 815 - DNA, 33 bp, Homo sapiens
taagctgcaattcttaggtgctttagagtgtaa

SEQ ID NO: 816 - DNA, 33 bp, Homo sapiens
taagctgcaattcttatgtgctttagagtgtaa

SEQ ID NO: 817 - DNA, 33 bp, Homo sapiens
aagggtgtgtaatagaatcctactccaacagga

SEQ ID NO: 818 - DNA, 33 bp, Homo sapiens
aagggtgtgtaatagactcctactccaacagga

SEQ ID NO: 819 - DNA, 33 bp, Homo sapiens
cagcccagaggaagacactgaagtccaaagagg

SEQ ID NO: 820 - DNA, 33 bp, Homo sapiens
cagcccagaggaagacgctgaagtccaaagagg

SEQ ID NO: 821 - DNA, 33 bp, Homo sapiens
acttggttgtcaggtaagtaagcaagaaattct

SEQ ID NO: 822 - DNA, 33 bp, Homo sapiens
acttggttgtcaggtaggtaagcaagaaattct

SEQ ID NO: 823 - DNA, 33 bp, Homo sapiens
ttagaggtatgacataaatgttctctttcactc

SEQ ID NO: 824 - DNA, 33 bp, Homo sapiens
ttagaggtatgacatagatgttctctttcactc

SEQ ID NO: 825 - DNA, 33 bp, Homo sapiens
taatccgatttcatcaactcacactaatcacta

SEQ ID NO: 826 - DNA, 33 bp, Homo sapiens
taatccgatttcatcagctcacactaatcacta

SEQ ID NO: 827 - DNA, 33 bp, Homo sapiens
ggacttttatcaggaaacaataaatgcatttca

SEQ ID NO: 828 - DNA, 33 bp, Homo sapiens
ggacttttatcaggaagcaataaatgcatttca

SEQ ID NO: 829 - DNA, 33 bp, Homo sapiens
attagctacaagtggacagtaaagcagggaact

SEQ ID NO: 830 - DNA, 33 bp, Homo sapiens
attagctacaagtggatagtaaagcagggaact

SEQ ID NO: 831 - DNA, 33 bp, Homo sapiens
agtgaaacacacataaatgagccatgcagagag

SEQ ID NO: 832 - DNA, 33 bp, Homo sapiens
agtgaaacacacataagtgagccatgcagagag

SEQ ID NO: 833 - DNA, 33 bp, Homo sapiens
tcaaaagacgtgcaaaacaccccaaaatcgttg

SEQ ID NO: 834 - DNA, 33 bp, Homo sapiens
tcaaaagacgtgcaaatcaccccaaaatcgttg

SEQ ID NO: 835 - DNA, 33 bp, Homo sapiens
aataagtgttctttacacaccaatggcttccta

SEQ ID NO: 836 - DNA, 33 bp, Homo sapiens
aataagtgttctttacgcaccaatggcttccta

SEQ ID NO: 837 - DNA, 33 bp, Homo sapiens
atggaagatggttaaacggggaaaattgcattc

SEQ ID NO: 838 - DNA, 33 bp, Homo sapiens
atggaagatggttaaatggggaaaattgcattc

SEQ ID NO: 839 - DNA, 33 bp, Homo sapiens
tacttctgaagcatgacgctagagtggaaagaa

SEQ ID NO: 840 - DNA, 33 bp, Homo sapiens
tacttctgaagcatgatgctagagtggaaagaa

SEQ ID NO: 841 - DNA, 33 bp, Homo sapiens
actttagtttttgccaactgcatatggaatgga

SEQ ID NO: 842 - DNA, 33 bp, Homo sapiens
actttagtttttgccagctgcatatggaatgga

SEQ ID NO: 843 - DNA, 33 bp, Homo sapiens
catgagaattcaataaactgagttatgccagtg

SEQ ID NO: 844 - DNA, 33 bp, Homo sapiens
catgagaattcaataatctgagttatgccagtg

SEQ ID NO: 845 - DNA, 33 bp, Homo sapiens
gtgcttaaacatatacaacgcattaaatgaagc

SEQ ID NO: 846 - DNA, 33 bp, Homo sapiens
gtgcttaaacatatacgacgcattaaatgaagc

SEQ ID NO: 847 - DNA, 33 bp, Homo sapiens
gcatagatagatggtaactatggaaacaatatg

SEQ ID NO: 848 - DNA, 33 bp, Homo sapiens
gcatagatagatggtagctatggaaacaatatg

SEQ ID NO: 849 - DNA, 33 bp, Homo sapiens
aatgttgccaatgcaaccaagaaagcctagagt

SEQ ID NO: 850 - DNA, 33 bp, Homo sapiens
aatgttgccaatgcaatcaagaaagcctagagt

SEQ ID NO: 851 - DNA, 33 bp, Homo sapiens
tttgtagtcttagaaccaggatgctttacattt

SEQ ID NO: 852 - DNA, 33 bp, Homo sapiens
tttgtagtcttagaactaggatgctttacattt

SEQ ID NO: 853 - DNA, 33 bp, Homo sapiens
tggtgggttccacctcaattgccaactttttag

SEQ ID NO: 854 - DNA, 33 bp, Homo sapiens
tggtgggttccacctcgattgccaactttttag

SEQ ID NO: 855 - DNA, 33 bp, Homo sapiens
cccaccattcagggtaaaagtttccacgccatc

SEQ ID NO: 856 - DNA, 33 bp, Homo sapiens
cccaccattcagggtagaagtttccacgccatc

SEQ ID NO: 857 - DNA, 33 bp, Homo sapiens
ctgttttctagagcaacgacatagggcagtgga

SEQ ID NO: 858 - DNA, 33 bp, Homo sapiens
ctgttttctagagcaatgacatagggcagtgga

SEQ ID NO: 859 - DNA, 33 bp, Homo sapiens
caactctttaccaaaaaaagtgaacataccaaa

SEQ ID NO: 860 - DNA, 33 bp, Homo sapiens
caactctttaccaaaagaagtgaacataccaaa

SEQ ID NO: 861 - DNA, 33 bp, Homo sapiens
ttattcccttattccactaatgcaaggcttaat

SEQ ID NO: 862 - DNA, 33 bp, Homo sapiens
ttattcccttattccagtaatgcaaggcttaat

SEQ ID NO: 863 - DNA, 33 bp, Homo sapiens
agcatcctgaatgacacagataaggaccatatt

SEQ ID NO: 864 - DNA, 33 bp, Homo sapiens
agcatcctgaatgacagagataaggaccatatt

SEQ ID NO: 865 - DNA, 33 bp, Homo sapiens
aaagacatgaaaggaaatgtagtttggtttgat

SEQ ID NO: 866 - DNA, 33 bp, Homo sapiens
aaagacatgaaaggaagtgtagtttggtttgat

SEQ ID NO: 867 - DNA, 33 bp, Homo sapiens
tcggtatgtgagcaaacgttaaatgacatagtg

SEQ ID NO: 868 - DNA, 33 bp, Homo sapiens
tcggtatgtgagcaaatgttaaatgacatagtg

SEQ ID NO: 869 - DNA, 33 bp, Homo sapiens
atcccaatacagactccgactaaggcttccctt

SEQ ID NO: 870 - DNA, 33 bp, Homo sapiens
atcccaatacagactctgactaaggcttccctt

SEQ ID NO: 871 - DNA, 33 bp, Homo sapiens
tgtaagcatcaggtaactcattcgaaagctaga

SEQ ID NO: 872 - DNA, 33 bp, Homo sapiens
tgtaagcatcaggtaattcattcgaaagctaga

SEQ ID NO: 873 - DNA, 33 bp, Homo sapiens
gtaagtagtttatgaagccctttctgatgatga

SEQ ID NO: 874 - DNA, 33 bp, Homo sapiens
gtaagtagtttatgaatccctttctgatgatga

SEQ ID NO: 875 - DNA, 33 bp, Homo sapiens
tagcctcagggcacagactgaaccaaaaaacag

SEQ ID NO: 876 - DNA, 33 bp, Homo sapiens
tagcctcagggcacaggctgaaccaaaaaacag

SEQ ID NO: 877 - DNA, 33 bp, Homo sapiens
gacggcagactacataacctgaaggacttcacc

SEQ ID NO: 878 - DNA, 33 bp, Homo sapiens
gacggcagactacataccctgaaggacttcacc

SEQ ID NO: 879 - DNA, 33 bp, Homo sapiens
cagcttacactggccacgtggtatcttgatgaa

SEQ ID NO: 880 - DNA, 33 bp, Homo sapiens
cagcttacactggccatgtggtatcttgatgaa

SEQ ID NO: 881 - DNA, 33 bp, Homo sapiens
caggtactgaactcccagctcctctacagcctg

SEQ ID NO: 882 - DNA, 33 bp, Homo sapiens
caggtactgaactcccggctcctctacagcctg

SEQ ID NO: 883 - DNA, 33 bp, Homo sapiens
aaatttggccacagaaactggctaggaaaaaac

SEQ ID NO: 884 - DNA, 33 bp, Homo sapiens
aaatttggccacagaagctggctaggaaaaaac

SEQ ID NO: 885 - DNA, 33 bp, Homo sapiens
gacaagggaaagttccaagaagagtgatacctc

SEQ ID NO: 886 - DNA, 33 bp, Homo sapiens
gacaagggaaagttcccagaagagtgatacctc

SEQ ID NO: 887 - DNA, 33 bp, Homo sapiens
tctattcactctaaaacggcagcagggttgagt

SEQ ID NO: 888 - DNA, 33 bp, Homo sapiens
tctattcactctaaaatggcagcagggttgagt

SEQ ID NO: 889 - DNA, 33 bp, Homo sapiens
aaggtcagcactgtcacgtaaggtatgtgctga

SEQ ID NO: 890 - DNA, 33 bp, Homo sapiens
aaggtcagcactgtcatgtaaggtatgtgctga

SEQ ID NO: 891 - DNA, 33 bp, Homo sapiens
ttttaaggtctttcgccgattgaatcagaccca

SEQ ID NO: 892 - DNA, 33 bp, Homo sapiens
ttttaaggtctttcgctgattgaatcagaccca

SEQ ID NO: 893 - DNA, 33 bp, Homo sapiens
taaagcagattaaagaatatttggcttttgttt

SEQ ID NO: 894 - DNA, 33 bp, Homo sapiens
taaagcagattaaagagtatttggcttttgttt

SEQ ID NO: 895 - DNA, 33 bp, Homo sapiens
ccccaaagacaacctacatgagtgaagagccaa

SEQ ID NO: 896 - DNA, 33 bp, Homo sapiens
ccccaaagacaacctagatgagtgaagagccaa

SEQ ID NO: 897 - DNA, 33 bp, Homo sapiens
aagggacagaatggaagagagagtatcagagta

SEQ ID NO: 898 - DNA, 33 bp, Homo sapiens
aagggacagaatggaatagagagtatcagagta

SEQ ID NO: 899 - DNA, 33 bp, Homo sapiens
tactctcaaaatgaccggggtggtgaatcgaac

SEQ ID NO: 900 - DNA, 33 bp, Homo sapiens
tactctcaaaatgacctgggtggtgaatcgaac

SEQ ID NO: 901 - DNA, 33 bp, Homo sapiens
ctagtaaatacaaaaaggacctaatgcttaaca

SEQ ID NO: 902 - DNA, 33 bp, Homo sapiens
ctagtaaatacaaaaatgacctaatgcttaaca

SEQ ID NO: 903 - DNA, 33 bp, Homo sapiens
gttgaagagtcgaaagcaataaatggtggccca

SEQ ID NO: 904 - DNA, 33 bp, Homo sapiens
gttgaagagtcgaaagtaataaatggtggccca

SEQ ID NO: 905 - DNA, 33 bp, Homo sapiens
cccagatcatatttcaagtccagtaacacagga

SEQ ID NO: 906 - DNA, 33 bp, Homo sapiens
cccagatcatatttcatgtccagtaacacagga

SEQ ID NO: 907 - DNA, 33 bp, Homo sapiens
tctgtgagaatagaaaatgaatcttttgggaag

SEQ ID NO: 908 - DNA, 33 bp, Homo sapiens
tctgtgagaatagaaagtgaatcttttgggaag

SEQ ID NO: 909 - DNA, 33 bp, Homo sapiens
taggatgcttttgcagaaattaacttgagtgga

SEQ ID NO: 910 - DNA, 33 bp, Homo sapiens
taggatgcttttgcagtaattaacttgagtgga

SEQ ID NO: 911 - DNA, 33 bp, Homo sapiens
actgagtcccaatgggcaactgattgcctcttt

SEQ ID NO: 912 - DNA, 33 bp, Homo sapiens
actgagtcccaatgggtaactgattgcctcttt

SEQ ID NO: 913 - DNA, 33 bp, Homo sapiens
tcaaatcaacaatctcactacacactcttatca

SEQ ID NO: 914 - DNA, 33 bp, Homo sapiens
tcaaatcaacaatctcgctacacactcttatca

SEQ ID NO: 915 - DNA, 33 bp, Homo sapiens
aatagtcatagcagaaataatactgataatatt

SEQ ID NO: 916 - DNA, 33 bp, Homo sapiens
aatagtcatagcagaagtaatactgataatatt

SEQ ID NO: 917 - DNA, 33 bp, Homo sapiens
atctctaactggtgataaagcatcatgaaattc

SEQ ID NO: 918 - DNA, 33 bp, Homo sapiens
atctctaactggtgatgaagcatcatgaaattc

SEQ ID NO: 919 - DNA, 33 bp, Homo sapiens
tccctgtctttggacacgtaagaattggaggaa

SEQ ID NO: 920 - DNA, 33 bp, Homo sapiens
tccctgtctttggacatgtaagaattggaggaa

SEQ ID NO: 921 - DNA, 33 bp, Homo sapiens
aacagaaaattaagataagattggctaacaaga

SEQ ID NO: 922 - DNA, 33 bp, Homo sapiens
aacagaaaattaagatcagattggctaacaaga

SEQ ID NO: 923 - DNA, 33 bp, Homo sapiens
aatgtaggtccataaaatgtttgcattgatcca

SEQ ID NO: 924 - DNA, 33 bp, Homo sapiens
aatgtaggtccataaactgtttgcattgatcca

(19) United States
(12) Patent Application Publication
(10) Pub. No.: US 2014/0023635 A1
(43) Pub. Date: Jan. 23, 2014

(54) SINGLE NUCLEOTIDE POLYMORPHISMS AND GENES ASSOCIATED WITH T2D-RELATED COMPLICATIONS

(71) Applicants: Pavel Hamet, Montreal (CA); Johanne Tremblay, Montreal (CA); Ondrej Seda, Prague (CZ); Stephen Macmahon, Sydney (AU); John Chalmers, Sydney (AU)

(72) Inventors: Pavel Hamet, Montreal (CA); Johanne Tremblay, Montreal (CA); Ondrej Seda, Prague (CZ); Stephen Macmahon, Sydney (AU); John Chalmers, Sydney (AU)

(73) Assignee: PROGNOMIX, INC., Montreal, QC (CA)

(21) Appl. No.: 13/995,976

(22) PCT Filed: Dec. 21, 2011
(86) PCT No.: PCT/IB11/03274

§ 371 Date: Sep. 12, 2013

(30) Foreign Application Priority Data
Dec. 21, 2010 (US) 61425748

(51) Int. Cl. C12Q 1/68

(52) U.S. Cl. CPC C12Q 1/6883
USPC 424/130.1; 435/6.11; 514/44 R; 506/9; 506/16; 514/44 A; 514/6.9

(57) ABSTRACT

The invention provides with means to predict, in subjects affected by type-2-diabetes (T2D), the probability of developing complications related to the disease. The invention involves (1) identification of genetic features such as single nucleotide polymorphisms (SNPs) for the establishment of a patient profile that can be used for prediction of complications associated with T2D. Signature profiles comprising a combination of SNPs which have greater predictive value for prognosticating particular types of complications, such as, stroke, myocardial infarction and kidney complications associated with T2D are further described. Compositions and kits that can be used with a set of complementary phenotypic markers to evaluate the risk for an individual affected by T2D to develop complications related to the disease and to evaluate the likelihood that an individual affected by type 2 diabetes type will benefit from treatments that collectively aim to reduce the risk of developing such complications.

The instant application claims priority to U.S. provisional application Ser. No. 61/425,748, filed Dec. 21, 2010, the disclosure in which is incorporated by reference in its entirety.

The invention provides means and methods to predict, in subjects affected by type II diabetes (T2D), the probability of developing complications related to the disease.

Diabetes mellitus is a heterogeneous group of metabolic diseases which is characterized by elevated blood glucose levels and increased morbidity. The endocrine cells of the pancreas which synthesize insulin and other hormones are involved in the pathogenesis of diabetes. Both genetic and environmental factors contribute to its development. The most common form is T2D, which is characterized by defects in both insulin secretion and insulin action. In contrast, type I diabetes results from autoimmune destruction of the insulin-producing beta cells of the pancreas. Monogenic forms of diabetes account for less than 5% of the cases and are usually caused by mutations in genes associated with maturity-onset diabetes of the young (MODY), insulin gene and insulin receptor gene.

T2D is a heterogeneous disease resulting from the interaction of environmental factors such as obesity or sedentary lifestyle, with variety of diabetogenic genes. Abnormal glucose homeostasis occurs when either insulin sensitivity or insulin secretion or both are altered. An early finding in this development is insulin resistance, defined as impaired insulin-mediated glucose clearance in insulin-sensitive tissues (skeletal muscle, liver and adipose tissue). Elevation of glucose levels triggers beta-cells to produce and secrete more insulin, which compensates for the disturbance in glucose homeostasis. The duration of hyperglycemia-hyperinsulinemia state depends on insulin secretory capacity, mass and apoptosis rate of beta-cells. Furthermore, beta-cells can loose their insulin secretion capacity because of glucose toxicity or other reasons. When cells fail to compensate for insulin resistance blood glucose concentration increases. Thus, over time subclinical hyperglycemia tends to progress to impaired glucose tolerance and further to T2D.

The causes of T2D are multi-factorial and include both genetic and environmental elements that affect beta cell function and insulin sensitivity of peripheral tissues (muscle, liver, adipose tissue, pancreas). Although there is considerable debate as to the relative contributions of beta-cell dysfunction and reduced insulin sensitivity to the pathogenesis of diabetes, it is generally agreed that both of these factors play important roles. Both impaired insulin secretion and insulin action cause the development of T2D. Insulin resistance is an early feature in the pathophysiology of T2D. No major single gene explaining the development of T2D has been identified. There were several studies attempting to predict T2D based on limited number of SNPs (up to 18) [Lyssenko, V, P Almgren, D Anevski et al.: Genetic prediction of future type 2 diabetes. PLoS Med 2, e345 (2005); Meigs, J B, P Shrader, LM Sullivan et al.: Genotype score in addition to common risk factors for prediction of type 2 diabetes. N Engl J Med 359, 2208-19 (2008); Cauchi, S, D Meyre, E Durand et al.: Post genome-wide association studies of novel genes associated with type 2 diabetes show gene-gene interaction and high predictive value. PLoS ONE 3, e2031 (2008). Miyake, K, W Yang, K Hara et al.: Construction of a prediction model for type 2 diabetes mellitus in the Japanese population based on 11 genes with strong evidence of the association. J Hum Genet 54, 236-41 (2009); Lin, X, K Song, N Lim et al.: Risk prediction of prevalent diabetes in a Swiss population using a weighted genetic score—the CoLaus Study. Diabetologia 52, 600-8 (2009)] but the reached predictive power was generally limited. Indeed, the mathematical models reflecting the presumable allele frequencies and risk effects estimate that for successful prediction, hundreds of SNPs will be necessary [(Kraft, P, D J Hunter: Genetic Risk Prediction—Are We There Yet? N Engl J Med 360, 1701-1703 (2009)].

Complications Associated with Diabetes

Over 90% of people diagnosed with diabetes have T2D, which carries a number of potential complications. These complications currently add very significantly to the cost of treating diabetes, because there is no reliable way to determine which patients are likely to develop such difficulties. Half of the people affected by T2D die from complications resulting from the disease.

Such Complications Include, but are not Limited to:

Cardiovascular Disease

Cardiovascular disease is the overwhelming cause of diabetes-related deaths. With the risk for stroke or myocardial infarction elevated by 2 to 4 times in persons with diabetes, a 65% majority of deaths among people with diabetes occurs from heart disease or stroke, considered as major macrovascular complications.

Diabetic Nephropathy

End-stage renal disease (ESRD) occurs when the kidneys cease to function, which ultimately leads to the need for a transplant or regular dialysis, both extremely costly procedures. Diabetes is responsible for 43% of the cases of ESRD as a consequence of microvascular damage of the kidney.

Diabetic Retinopathy

Diabetes is also the leading cause of blindness in people aged 20-74. Diabetic retinopathy is considered as one type of microvascular complication and is responsible for over 24,000 cases of blindness in the United States.

Diabetic Neuropathy

It is estimated that over 70% of people with diabetes may also suffer from nervous system damage, causing impaired sensation or pain in the feet or hands, slowed digestion of food in the stomach, carpal tunnel syndrome, and other nerve problems. In the severe cases of diabetic neuropathy, usually combined with peripheral vessel macro and microvascular disease, patients may have to undergo lower-extremity amputations.

Dental disease, complications of pregnancy, coma, and acute susceptibility to opportunistic infectious diseases are also costly diabetes-related diseases.

Drugs designed to prevent or stabilize complications are extremely costly. Because of the debilitating effects of diabetes-related complications, healthcare professionals are forced to prescribe costly medications to diabetes patients to protect them against developing these complications without having any efficient and reliable mean to predict those patients who will develop these complications and the efficiency of these treatments.

There is a need for assays capable of identifying among T2D patients those who are at risk of developing complications and those who would benefit from the various treatments available. The present invention provides with means to develop such assays and to utilize them in a clinical and medical environment.

Polymorphisms

DNA polymorphisms provide an efficient way to study the association of genes and diseases by analysis of linkage and linkage disequilibrium. With the sequencing of the human genome a myriad of hitherto unknown genetic polymorphisms among people have been detected. Most common among these are the single nucleotide polymorphisms, also called SNPs, of which there are known several millions. Other examples are short tandem repeat polymorphisms (STR), variable number of tandem repeat polymorphisms (VNTR), insertions, deletions and block modifications. Tandem repeats (STR or VNTR) often have multiple different alleles (variants) in population, whereas the other groups of polymorphisms usually have just two alleles. Some of these genetic polymorphisms play a direct role in the biology of the individuals, including their risk of developing disease, but the virtue of the majority is that they can serve as markers for the surrounding DNA. The relationship of an allele of one sequence polymorphism with particular alleles of other sequence polymorphisms in the surrounding is due to phenomenon called genetic linkage. Linkage arises because large parts of chromosomes are passed unchanged from parents to offspring, so that minor regions of a chromosome tend to flow unchanged from one generation to the next and also to be similar in different branches of the same family. Linkage is gradually eroded by recombination occurring in the germline cells, but typically operates over multiple generations and distances of a number of million bases in the DNA.

Linkage disequilibrium deals with whole populations and has its origin in the (distant) forefather in whose DNA a new sequence polymorphism arose. The immediate surroundings in the DNA of the forefather will tend to stay with the new allele and propagate together to the offspring for many generations. Recombination and changes in the composition of the population will again erode the association, but the new allele and the alleles of any other polymorphism nearby will often be partly associated among unrelated humans even today. A crude estimate suggests that alleles of sequence polymorphisms with distances less than 10000 bases in the DNA will have tended to stay together since modern man arose. Linkage disequilibrium in limited populations, for instance Europeans, often extends over longer distances, e.g. over more than 1,000,000 bases. This can be the result of newer mutations, but can also be a consequence of one or more “bottlenecks” with small effective population sizes and considerable inbreeding in the history of the current population. Two obvious possibilities for “bottlenecks” in Europeans are the exodus from Africa and the repopulation of Europe after the last ice age. A number of polymorphisms have been associated with induction of exocrine pancreatic dysfunction and/or diabetes. Some of the identified polymorphisms have been suggested in patent literature as useful in diagnosis of diabetes (see for example WO9321343 related to polymorphisms in GCK gene, and WO0023591 related to polymorphism in ZSIG49 gene).

Linkage disequilibrium can be quantitatively determined by measures such as r2 and |D′|. The SNPs of the instant application which are in linkage disequilibrium with the recited SNPs may be at an r2 threshold of at least 0.5, at least 0.6, at least 0.7, at least 0.8, at least 0.9, at least 0.95, at least 0.99. As for the parameters used in the calculation of LD, such are known in the art. See for example, the textbook on Genomic and Personalized Medicine, Volume 1: Principles, Methodology and translational Approaches, edited by Huntington F. Willard and Geoffrey S. Ginsburg, Institute for Genome Sciences and Policies, Duke University, Durham, N.C., USA; Academic Press, which is incorporated by reference herein in its entirety.

Up to date, several genomic regions or individual genetic variants have been found to be linked or associated to the phenotypes closely related to diabetic complications.

Linkage Studies

In a recent study (Diabetes. 2008 January; 57(1):235-43), a genome-wide scan for estimated glomerular filtration rate was performed in multi-ethnic diabetic populations (the Family Investigation of Nephropathy and Diabetes (FIND)) using 404 STR markers. For all ethnicities combined, strong evidence for linkage was observed on chromosomes 1q43, 7q36.1, 8q13.3 and 18q23.3. Mexican-American families, who comprised the major ethnic subpopulation in FIND, contributed to linkage on chromosomes 1q43, 2p13.3, 7q36.1, 8q13.3, and 18q23.3, whereas African-American and American-Indian families displayed linkage peaks on chromosomes 11p15.1 and 15q22.3, respectively. Also in FIND study (Diabetes. 2007 June; 56(6):1577-85), the strongest evidence of linkage to the diabetic nephropathy trait was on chromosomes 7q21.3, 10p15.3, 14q23.1, and 18q22.3. In ACR (883 diabetic sibling pairs), the strongest linkage signals were on chromosomes 2q14.1, 7q21.1, and 15q26.3. These results confirm regions of linkage to diabetic nephropathy on chromosomes 7q, 10p, and 18q from prior reports. In Mexican Americans, Puppala al. (Diabetes. 2007 November; 56 (11):2818-28) found a linkage of glomerular filtration rate to a region on chromosome 2q near marker D2S427 (corrected LOD score 3.3), influenced by genotype by diabetes interaction.

Association Studies

A number of genes and genetic polymorphisms were tested for their association to diabetic nephropathy, either because of their reported relevance in metabolic and signaling pathways connected to pathophysiology of diabetic complications (functional candidates) or combination of the former with their genomic position under peak of ascertained linkage (positional candidates), or as a result of genome-wide association studies. Genes for which an association was found with diabetic nephropathy include 5,10-methylenetetrahydrofolate reductase (MTHFR), natriuretic peptide precursor A (NPPA), solute carrier family 2 member 1 (facilitated glucose transporter SLC2A1), lamin A/C (LMNA), retinoid X receptor gamma (RXRG), interleukin 1 receptor antagonist (IL1RN), ghrelin/obestatin preprohormone (GHRL), peroxisome proliferator-activated receptor gamma (PPARG), chemokine receptor 5 (CCR5), angiotensin II receptor type 1 (AGTR1), solute carrier family 2 member 2 (facilitated glucose transporter SLC2A2), adiponectin (ADIPOQ), fatty acid binding protein 2 (FABP2), glutamine-fructose-6-phosphate transaminase 2 (GFPT2), advanced glycosylation end product-specific receptor (AGER), lymphotoxin alpha (LTA), vascular endothelial growth factor A (VEGF), ectonucleotide pyrophosphatase/phosphodiesterase 1 (ENPP1), SMT3 suppressor of mif two 3 homolog 4 (small ubiquitin-like modifier 4 protein SUMO4), estrogen receptor 1 (ESR1), superoxide dismutase 2 (SOD2), neuropeptide Y (NPY), engulfment and cell motility 1 (ELMO1), insulin-like growth factor binding protein 1 (IGFBP1), epidermal growth factor receptor (EGFR), paraoxonase 1 (PON1), aldo-keto reductase family 1, member B1 (AKR1B1), caldesmon 1 (CALD1), nitric oxide synthase 3 (NOS3), lipoprotein lipase (LPL), Pvt1 oncogene homolog MYC activator (PVT1), insulin (INS), xylosyltransferase I (XYLT), protein kinase C beta 1 (PRKCB1), solute carrier family 12 (SLC12A3), haptoglobin (HP), chemokine ligand 2 (CCL2), angiotensin I converting enzyme (ACE), meprin A, beta (MEP1B), carnosine dipeptidase 1 (CNDP1), intercellular adhesion molecule 1 (ICAM1), transforming growth factor beta 1 (TGFB1), apolipoprotein E (APOE) and superoxide dismutase 1 (SOD1), CD48 molecule (CD48), solute carrier family 35, member F3 (SLC35F3), endothelial PAS domain protein 1 (EPAS1), low density lipoprotein-related protein 1B (deleted in tumours) (LRP1B), dynein, axonemal, heavy chain 7 (DNAH7), ADAM metallopeptidase domain 23 (ADAM23), leprecan-like 1 (LEPREL1), human immunodeficiency virus type I enhancer binding protein 2 (HIVEP2), thrombospondin, type I, domain containing 7A (THSD7A), S-adenosylhomocysteine hydrolase-like 2 (AHCYL2), discs, large homolog 2 (Drosophila) (DLG2), FYVE, RhoGEF and PH domain containing 4 (FGD4), rabphilin 3A homolog (mouse) (RPH3A), citrate lyase beta like (CLYBL), protein kinase C, eta (PRKCH), epidermal growth factor receptor pathway substrate 15-like 1 (EPS15L1), cystatin 9 (testatin) (CST9), pericentrin (PCNT2), matrix metallopeptidase 9 (gelatinase B, 92 kDa gelatinase, 92 kDa type IV collagenase) (MMP9), apolipoprotein C-I (APOC1), cysteinyl-tRNA synthetase (CARS), chimerin (chimaerin) 2 (CHN2), neurocalcin delta (NCALD), ectonucleotide pyrophosphatase/phosphodiesterase 1 (ENPP1), major histocompatibility complex, class II, DR beta 1 (HLA-DRB1), interleukin 8 (IL8), pleckstrin homology domain containing, family H (with MyTH4 domain) member 2 (PLEKHH2), angiotensinogen (serpin peptidase inhibitor, Glade A, member 8) (AGT) and others, reviewed in Maeda, S: Genetics of diabetic nephropathy. Ther Adv Cardiovasc Dis 2, 363-(2008).

Genes and markers in type-2 diabetes and obesity are described in US patent application publication No. 2007-0292412 to Salonen et al.

The totality of disclosures in WO 09/150,550 as well as US patent application publication Nos. 2010-0099091 and 2010-0136540 are incorporated by reference herein.

Proteomic Studies

Few proteomic studies have been so far conducted with the aim of identifying biomarkers of predictive value for diabetic nephropathy. Otu et al. (Diabetes Care (2007) 30:638-543) (WO 2007/056587) performed a nested case-control study on Pima Indians that allowed for identification of a 12-peak proteomic signature that was validated on a small number of individuals. However, the study did not permit the identification of the proteins from which these peaks belong and replication in other populations is needed prior to concluding the broad applicability of these biomarkers.

Up to date, there have been dozens of genetic variants and genome regions associated or linked to myocardial infarction, both in T2D patients as well as in non-diabetics. These have been comprehensively reviewed recently (Yamada, Y, S Ichihara, T Nishida: Molecular genetics of myocardial infarction. Genomic Med 2, 7-22 (2008)) and specifically for genome-wide association studies in A Catalog of Published Genome-Wide Association Studies by The National Human Genome Research Institute (available on the world wide web at genome.gov/26525384) Johnson, A D, C J O'Donnell: An open access database of genome-wide association results. BMC Med Genet 10, 6 (2009).

### SUMMARY OF THE INVENTION

The present invention relates to previously unknown associations between T2D-related complications and various polymorphisms, genes and loci. These associated polymorphisms, genes, and loci provide basis for novel methods and kits for risk assessment, diagnosis and prognosis of T2D-related complication in a patient, among other things. In addition these polymorphisms, genes, and loci provide basis for methods and kits for novel therapies to prevent, treat and/or reduce risk of developing these complications.

A “biomarker” in the context of the present invention refers to a genetic feature such as, for example, single nucleotide polymorphism (SNP) or a short tandem repeat (STR). Other types of biomarkers include, but are not limited to, transcriptional products (such as, for example, mRNA or cDNA sequences thereof) or translational products (such as, for example, proteins or polypeptides) of genes comprising such SNPs. Representative examples of such SNPs include, but are not limited to, SNPs having the RefSNPID Nos. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544. These SNPs are collectively disclosed in Table E.

A combination of the aforementioned biomarkers may also be employed. The combination(s) employed herein may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264, at least 265, at least 266, at least 267, at least 268, at least 269, at least 270, at least 271, at least 272, at least 273, at least 274, at least 275, at least 276, at least 277, at least 278, at least 279, at least 280, at least 281, at least 282, at least 283, at least 284, at least 285, at least 286, at least 287, at least 288, at least 289, at least 290, at least 291, at least 292, at least 293, at least 294, at least 295, at least 296, at least 297, at least 298, at least 299, at least 300, at least 301, at least 302, at least 303, at least 304, at least 305, at least 306, at least 307, at least 308, at least 309, at least 310, at least 311, at least 312, at least 313, at least 314, at least 315, at least 316, at least 317, at least 318, at least 319, at least 320, at least 321, at least 322, at least 323, at least 324, at least 325, at least 326, at least 327, at least 328, at least 329, at least 330, at least 331, at least 332, at least 333, at least 334, at least 335, at least 336, at least 337, at least 338, at least 339, at least 340, at least 341, at least 342, at least 343, at least 344, at least 345, at least 346, at least 347, at least 348, at least 349, at least 350, at least 351, at least 352, at least 353, at least 354, at least 355, at least 356, at least 357, at least 358, at least 359, at least 360, at least 361, at least 362, at least 363, at least 364, at least 365, at least 366, at least 367, at least 368, at least 369, at least 370, at least 371, at least 372, at least 373, at least 374, at least 375, at least 376, at least 377, at least 378, at least 379, at least 380, at least 381, at least 382, at least 383, at least 384, at least 385, at least 386, at least 387, at least 388, at least 389, at least 390, at least 391, at least 392, at least 393, at least 394, at least 395, at least 396, at least 397, at least 398, at least 399, at least 400, at least 401, at least 402, at least 403, at least 404, at least 405, at least 406, at least 407, at least 408, at least 409, at least 410, at least 411, at least 412, at least 413, at least 414, at least 415, at least 416, at least 417, at least 418, at least 419, at least 420, at least 421, at least 422, at least 423, at least 424, at least 425, at least 426, at least 427, at least 428, at least 429, at least 430, at least 431, at least 432, at least 433, at least 434, at least 435, at least 436, at least 437, at least 438, at least 439, at least 440, at least 441, at least 442, at least 443, at least 444, at least 445, or at least 446 of the aforementioned biomarkers.

Combination of biomarkers. The instant invention also utilizes a combination of the biomarkers for improvement of predictive success/accuracy. Methods for combining risk alleles to create risk scores are known in the art. For example, Ortlepp et al. (Eur J Intern Med. 2002 December; 13(8):485-492) evaluate the benefit of analyzing multiple genetic polymorphisms as a compound unit to assess the individual genetic burden for coronary artery disease (CAD). The authors state that although “none of the polymorphisms showed a significant difference in the allele frequency between case and control patients . . . eight genes with a higher allele frequency in the case group (delta allele frequency >0.05) were defined as risk alleles (RA) and subsequently tested as a compound unit. A risk stratification for 64.5% of all patients was possible when eight genes with their 16 RA were included in the analysis. With more than eight RA per individual, the odds ratio for developing CAD was 3.21 (95% CI 1.77-5.82, P<0.001). However, there was still an overlap in the number of RA in case and control patients.” Using a computer simulation, the researchers determined that a combined analysis of >200 polymorphisms provides a reasonable genetic discrimination for patients at risk for CAD. Similar techniques can be employed in the instant case.

The instant invention also makes use of preferred types of SNPs in the meta-analysis. Representative methods for identification of these SNPs are provided in the Examples section. Such techniques that have been described in literature. To this end, de Miguel-Yanes et al. (Diabetes Care. 2011 January; 34(1):121-5) disclose the use of weighted risk scores, wherein risk alleles were counted (0, 1, 2) for each genotyped SNP—or its dosage when imputed—(actual distribution ranging from 28 to 53) are used. In one case, each SNP genotype was multiplied by its published β coefficient for diabetes risk. The product of that multiplication at each SNP was added and the sum was divided by twice the sum of the β coefficients, and the result was multiplied by the number of SNPs.

Preferably, the combinations of the instant invention comprise at least 50, at least 100, at least 150, at least 200 or more of the aforementioned biomarkers from Tables A-D.

The instant invention also provides for subsets of biomarkers that are useful in the prognostication of a type-2-diabetes related specific complication which is stroke, myocardial infarction, kidney-related complication (e.g., nephropathy or albuminurea) or a combination thereof.

As used herein, kidney complications associated with type-2 diabetes often manifests itself in terms of nephropathy and/or albuminuria. The resultant effect is declining glomerular filtration rate, which can be measured by skilled clinicians. The term “declining glomerular filtration,” as used herein, is defined in literature. For example, de Galan et al. (“Lowering blood pressure reduces renal events in type 2 diabetes,” J Am Soc Nephrol. 2009 April; 20(4):883-92; PMID: 19225038) note that declining glomerular filtration is characterized by “doubling of serum creatinine to >200 μmole/L, or end-stage kidney disease (defined as requirement for renal replacement therapy or renal death).” The disclosure in de Galan et al. is incorporated by reference herein.

In one such embodiment, the present invention provides a subset of biomarkers that are useful in prognostication of stroke in a T2D patient. This biomarker subset comprises SNPs having the RefSNP ID Nos. rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, and/or rs10189134. These SNPs are also recited in Table A attached hereto.

A combination of the aforementioned stroke-prognosticating biomarker subsets can also be employed. Such combinations comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, or more of the aforementioned biomarkers of Table A.

In another such embodiment, the present invention provides a subset of biomarkers that are useful in prognostication of myocardial infarction (MI) in a T2D patient. This biomarker subset comprises SNPs having the RefSNP ID Nos. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, and/or rs17191463. These SNPs are also recited in Table B attached hereto.

A combination of the aforementioned MI-prognosticating biomarker subsets can also be employed. Such combinations comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, or more of the aforementioned biomarkers of Table B.

In another such embodiment, the present invention provides a subset of biomarkers that are useful in prognostication of kidney-related complication in a T2D patient. This biomarker subset comprises SNPs having the RefSNP ID Nos. rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs3917768, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs7765427, rs963264, rs1258323, rs28490018, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs6814800, rs3909788, rs7690478, rs6810410, rs7549770, rs7935913, rs1161262, rs4497678, rs10155009, rs4258086, rs11927384, rs4269885, rs16825798, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs11848645, rs7203172, rs2171304, rs6585304, rs7967892, rs7501838, rs3883317, rs12973797, rs3809485, rs6589065, rs765, rs7535769, rs11688740, rs10514925, rs13333226, rs4702266, rs980907, rs17600042, rs9540649, rs634585, rs1327210, rs17680407, rs17680905, rs1913695, rs191606, rs11951359, rs10071774, rs10471462, rs617595, rs7195806, rs4940275, rs16880395, rs7748736, rs9293123, rs9502593, rs17078168, rs9287229, rs7243021, rs2936535, rs11183154, rs2324872, rs1218661, rs12511845, rs11752805, rs11707973, rs2740485, rs4936167, rs4572098, rs243839, rs2962394, rs7334517, rs130250, rs4378283, rs1364406, rs10153433, rs10248275, and/or rs1519729. These SNPs biomarkers are also recited in Table C attached hereto.

A combination of the aforementioned kidney complication-prognosticating biomarker subsets can also be employed. Such combinations comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, or more of the aforementioned biomarkers of Table C.

The present invention also provides biomarkers that are of prognostic significance for any complication associated with type-2-diabetes, such as, for example, stroke, myocardial infarction and/or kidney related complications. Such biomarkers include SNPs having the RefSNP ID Nos. rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544. These SNPs biomarkers are also recited in Table D attached hereto.

A combination of the aforementioned prognosticating biomarkers for T2D complications can also be employed. Such combinations comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, or more of the aforementioned biomarkers of Table D.

The aforementioned prognosticating biomarkers of Table D may be utilized solely or in combination with one or more biomarkers from Table A, Table B, and/or Table C. For example, if one skilled in the art is interested in prognosticating stroke in a patient with T2D, a combination of the biomarkers in Table A and Table D may be employed. Similarly, for prognostication of myocardial infarction, a combination of biomarkers from Table B and Table D may be employed. Moreover, for the prognostication of kidney-related complications in T2D subjects, a combination of the biomarkers in Table C and Table D may be employed.

Polymorphic genes of the present invention comprise the genes/loci also disclosed in Table G A “biomarker” can also be a clinical or biological biomarker. Clinical or biological biomarkers include, but are not limited to, age, sex, glucose levels, age of diagnosis, diabetes duration at baseline, cigarette smoking, diastolic or systolic blood pressure, atrial fibrillation, glycated hemoglobin (HbA1_{c}), total cholesterol, HDL cholesterol, albumin/creatinine ratio, glomerular filtration rate.

In one embodiment, the biomarker is a T2D-complication related biomarker which is

(a) a SNP having the RefSNPID Nos. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) a SNP or a STR found to be in linkage disequilibrium to one of the SNP listed in (a).

In another embodiment, the biomarker is a stroke-associated biomarker which is

(a) a SNP having the RefSNPID Nos. rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, and/or rs10189134; or
(b) a SNP or a STR found to be in linkage disequilibrium to one of the SNP listed in (a).

In another embodiment, the biomarker is a MI-associated biomarker which is

(a) a SNP having the RefSNPID Nos. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, and/or rs17191463; or
(b) a SNP or a STR found to be in linkage disequilibrium to one of the SNP listed in (a).

In another embodiment, the biomarker is a kidney complication-associated biomarker which is

(a) a SNP having the RefSNPID Nos. rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs3917768, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs7765427, rs963264, rs1258323, rs28490018, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs6814800, rs3909788, rs7690478, rs6810410, rs7549770, rs7935913, rs1161262, rs4497678, rs10155009, rs4258086, rs11927384, rs4269885, rs16825798, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs11848645, rs7203172, rs2171304, rs6585304, rs7967892, rs7501838, rs3883317, rs12973797, rs3809485, rs6589065, rs765, rs7535769, rs11688740, rs10514925, rs13333226, rs4702266, rs980907, rs17600042, rs9540649, rs634585, rs1327210, rs17680407, rs17680905, rs1913695, rs191606, rs11951359, rs10071774, rs10471462, rs617595, rs7195806, rs4940275, rs16880395, rs7748736, rs9293123, rs9502593, rs17078168, rs9287229, rs7243021, rs2936535, rs11183154, rs2324872, rs1218661, rs12511845, rs11752805, rs11707973, rs2740485, rs4936167, rs4572098, rs243839, rs2962394, rs7334517, rs130250, rs4378283, rs1364406, rs10153433, rs10248275, and/or rs1519729; or
(b) a SNP or a STR found to be in linkage disequilibrium to one of the SNP listed in (a).

In another embodiment, the biomarker is associated with any T2D-complication which is stroke, MI and/or kidney-complication, wherein said biomarker is

(a) a SNP having the RefSNPID Nos. rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) a SNP or a STR found to be in linkage disequilibrium to one of the SNP listed in (a).

Preferably, the biomarker is not a SNP having the RefSNP ID listed in Table F.

In another embodiment, the biomarker is a SNP of at least one of the genes listed in Table G or a STR linked to a SNP of at least one of these above genes or to a locus closely related thereto.

The present invention thus provides for methods of predicting risk of complications associated with T2D, comprising detecting (a) at least one SNP having the RefSNPID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or (b) a SNP or a STR found to be in linkage disequilibrium to one of the SNP listed in (a); wherein the presence of the SNP or STR in a sample of a subject (or patient) suffering from T2D indicates that said subject (or patient) is likely to develop the complication. Preferred examples of such complications include, but are not limited to, albuminuria and/or declining glomerular filtration, myocardial infarction, and/or stroke.

Any combination of the aforementioned SNP biomarkers may be detected.

In another embodiment, the present invention provides methods for predicting risk of stroke associated with T2D, comprising detecting

(a) a SNP having the RefSNPID Nos. rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, and/or rs10189134; or
(b) a SNP or a STR found to be in linkage disequilibrium to one of the SNP listed in (a);
wherein the presence of the SNP or STR in a sample of a subject (or patient) suffering from T2D indicates that said subject (or patient) is likely to develop said stroke.

Any combination of the aforementioned SNP biomarkers may be detected.

In another embodiment, the present invention provides methods for predicting risk of MI-associated with T2D, comprising detecting

(a) a SNP having the RefSNPID Nos. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, and/or rs17191463; or
(b) a SNP or a STR found to be in linkage disequilibrium to one of the SNP listed in (a);
wherein the presence of the SNP or STR in a sample of a subject (or patient) suffering from T2D indicates that said subject (or patient) is likely to develop said MI.

Any combination of the aforementioned SNP biomarkers may be detected.

In another embodiment, the present invention provides methods for predicting risk of kidney complications in a subject (or patient) with T2D, comprising detecting

(a) a SNP having the RefSNPID Nos. rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs3917768, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs7765427, rs963264, rs1258323, rs28490018, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs6814800, rs3909788, rs7690478, rs6810410, rs7549770, rs7935913, rs1161262, rs4497678, rs10155009, rs4258086, rs11927384, rs4269885, rs16825798, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs11848645, rs7203172, rs2171304, rs6585304, rs7967892, rs7501838, rs3883317, rs12973797, rs3809485, rs6589065, rs765, rs7535769, rs11688740, rs10514925, rs13333226, rs4702266, rs980907, rs17600042, rs9540649, rs634585, rs1327210, rs17680407, rs17680905, rs1913695, rs191606, rs11951359, rs10071774, rs10471462, rs617595, rs7195806, rs4940275, rs16880395, rs7748736, rs9293123, rs9502593, rs17078168, rs9287229, rs7243021, rs2936535, rs11183154, rs2324872, rs1218661, rs12511845, rs11752805, rs11707973, rs2740485, rs4936167, rs4572098, rs243839, rs2962394, rs7334517, rs130250, rs4378283, rs1364406, rs10153433, rs10248275, and/or rs1519729; or
(b) a SNP or a STR found to be in linkage disequilibrium to one of the SNP listed in (a);
wherein the presence of the SNP or STR in a sample of a subject (or patient) suffering from T2D indicates that said subject (or patient) is likely to develop said kidney complication.

Any combination of the aforementioned SNP biomarkers may be detected.

In another embodiment, the present invention provides methods for predicting risk of developing any T2D-complication which is stroke, MI and/or kidney-complication, wherein said biomarker is

(a) a SNP having the RefSNPID Nos. rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) a SNP or a STR found to be in linkage disequilibrium to one of the SNP listed in (a);
wherein the presence of the SNP or STR in a sample of a subject (or patient) suffering from T2D indicates that said subject (or patient) is likely to develop said T2D-complication.

Any combination of the aforementioned SNP biomarkers may be detected.

Preferably, the method does not comprise detecting a SNP having the RefSNP ID shown in Table F.

As used herein, “single nucleotide polymorphism,” or “SNP” is a DNA sequence variation that occurs when a nucleotide, e.g., adenine (A), thymine (T), cytosine (C), or guanine (G), in the genome sequence is altered to another nucleotide. SNPs are occasional variations in DNA sequence; the vast majority of the DNA sequence is identical among all humans. SNPs or other variants may also be found in genomic regions that do not contain genes. They represent a genomic hot spot responsible for the genetic variability among humans.

As used herein, “gene” means any amount of nucleic acid material that is sufficient to encode a transcript or protein having the function desired. Thus, it includes, but is not limited to, genomic DNA, cDNA, RNA, and nucleic acid that are otherwise genetically engineered to achieve a desired level of expression under desired conditions. Accordingly, it includes fusion genes (encoding fusion proteins), intact genomic genes, and DNA sequences fused to heterologous promoters, operators, enhancers, and/or other transcription regulating sequences. Methods and nucleic acid constructs for preparing genes for recombinant expression are well known and widely used by those of skill in the art, and thus need not be detailed here. The term refers to an entirety containing entire transcribed region and all regulatory regions of a gene. The transcribed region of a gene including all exon and intron sequences of a gene including alternatively spliced exons and introns so the transcribed region of a gene contains in addition to polypeptide encoding region of a gene also regulatory and 5′ and 3′ untranslated regions present in transcribed RNA.

The genes of the invention are listed in Table G.

As used herein, an “exon” is a segment of a eukaryotic gene that encodes a sequence of nucleotides in mRNA. An exon can encode amino acids in a protein. Exons are generally adjacent to introns.

As used herein, an “intron” is a non-coding region of a eukaryotic gene that may be transcribed into an RNA molecule, but is not usually translated into amino acids. It may be excised by RNA splicing when mRNA is produced.

As used herein, a “patient” is any living animal, including, but not limited to, a human who has, or is suspected of having or being susceptible to, a disease or disorder, or who otherwise would be a subject of investigation relevant to a disease or disorder. Accordingly, a patient can be an animal that has been bred or engineered as a model for metabolic syndrome, type 2 diabetes, obesity, hypertension, atherosclerosis, or any other disease or disorder Likewise it can be a human suffering from, or at risk of developing, a disease or disorder associated with insulin metabolism, or any other disease or disorder. Similarly, a patient can be an animal (such as an experimental animal, a pet animal, a farm animal, a dairy animal, a ranch animal, or an animal cultivated for food or other commercial use), or a human, serving as a healthy control for investigations into diseases and/or disorders, e.g., those associated with insulin metabolism.

By “reagent,” is meant any element, molecule, or compound that is present in the assay system and participates, either directly or indirectly, in the biochemical processes occurring during the performance of the method. Reagents include, but are not limited to, nucleic acids, cells, media, chemicals, compounds used to introduce nucleic acids into cells, and compounds used to generate detectable signals.

By “materials” is meant items that are used to contain and/or perform the methods of the invention, but that do not participate in any of the biochemical reactions taking place in the method. Materials include, but are not limited to, test tubes, pipettes, gels, and ultraviolet transilluminators.

A “haplotype,” as described herein, refers to any combination of genetic markers (“alleles”) usually inherited together. A haplotype can comprise two or more alleles and the length of a genome region comprising a haplotype may vary from few hundred bases up to hundreds of kilobases. As it is recognized by those skilled in the art, the same haplotype can be described differently by determining the haplotype defining alleles from different nucleic acid strands. For example, the haplotype GGC defined by the SNP markers of this invention is the same as haplotype CCG in which the alleles are determined from the other strand, or haplotype CGC, in which the first allele is determined from the other strand. The haplotypes described herein are differentially present in T2D patients with increased risk of developing one or more of the aforementioned complications. Therefore, these haplotypes have diagnostic value for risk assessment, diagnosis and prognosis of T2D-related complications. Detection of haplotypes can be accomplished by methods known in the art used for detecting nucleotides at polymorphic sites.

A nucleotide position in genome at which more than one sequence is possible in a population, is referred to herein as a “polymorphic site” or “polymorphism”. Where a polymorphic site is a single nucleotide in length, the site is referred to as a SNP. For example, if at a particular chromosomal location, one member of a population has an adenine and another member of the population has a cytosine at the same position of his or her paternal or maternal DNA molecule, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a cytosine allele. Typically, a reference nucleotide sequence is referred to for a particular polymorphism e.g. in NCBI databases (as accessible on the world-wide-web at ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences. Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by the genes comprising such SNPs. For example, a nucleotide change resulting in a change in polypeptide sequence can alter the physiological properties of a polypeptide dramatically by resulting in altered activity, distribution and stability or otherwise affect on properties of a polypeptide. Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of a susceptibility gene, such as, for example, an SNP associated with the aforementioned genes.

The representative examples in the instant specification describe detection of minor alleles of SNPs and provides odds ratio (OR) associated with these minor alleles. Although this is a preferred embodiment, the presently claimed invention can be routinely practiced via detection of the major allele of any particular SNP. To this end, the skilled worker understands that the total frequency of major and minor allele in a population is 100%. In the instant application, wherein the minor allele frequency is higher in cases than in controls (OR>1), the minor allele is a risk allele while wherein the minor allele frequency is higher in controls than in cases (OR<1), the major allele is a risk allele. Not only is it art-accepted in genome-wide association studies to calculate odds ratio (OR) for the minor allele, but the specification explicitly discloses the use of PLINK. See Purcell et al. (“PLINK: a tool set for whole-genome association and population-based linkage analyses.” American Journal of Human Genetics 81(3): 559-575, 2007), which is incorporated by reference herein in its entirety. By default PLINK uses the minor allele as the reference allele as shown in information publicly available on this computer program.

The SNP markers of the present invention, which are disclosed in Tables A, B, C, D, E and F have been denoted with their official reference SNP (rs) ID identification tags assigned to each unique SNP by the National Center for Biotechnological Information (NCBI). Each rs ID has been linked to specific variable alleles present in a specific nucleotide position in the human genome, and the nucleotide position has been specified with the nucleotide sequences flanking each SNP.

Although the numerical chromosomal position of a SNP may still change upon annotating the current human genome build the SNP identification information such as variable alleles and flanking nucleotide sequences assigned to a SNP will remain the same. Those skilled in the art will readily recognize that the analysis of the nucleotides present in one or more SNPs set forth in tables A-F of this invention in an individual's nucleic acid can be done by any method or technique capable of determining nucleotides present in a polymorphic site using the sequence information assigned in prior art to the rs IDs of the SNPs listed in tables A-F of this invention. As it is obvious in the art the nucleotides present in polymorphisms can be determined from either nucleic acid strand or from both strands.

In one embodiment, the invention relates to a method for predicting the risk of developing a complication which is albuminuria and/or declining glomerular filtration myocardial infarction, or stroke in a subject having T2D, comprising detecting in a sample obtained from said subject at least one SNP having an RefSNP ID listed in Tables A-D.

In a related aspect, the present invention relates to a method for predicting the risk of developing a complication which is either myocardial infarction, or stroke or albuminuria and/or declining glomerular filtration or any combination thereof in a subject having T2D, comprising detecting at least one SNP having an RefSNP ID listed of in Tables A-D.

In a related aspect, the present invention relates to a method for predicting the risk of developing a complication which is albuminuria and/or declining glomerular filtration myocardial infarction, or stroke in a subject having T2D, comprising detecting in a sample obtained from said subject at least one SNP having an RefSNP ID listed in Tables A-D, wherein said RefSNP ID is not listed in Table F.

Determination of Repeated Sequences

The present invention also provides a method for prognosticating T2D-related complication in a subject comprising detecting short tandem repeats (STR) in linkage disequilibrium with a SNP listed in Tables A-D. The present invention thus provides for methods of predicting risk of complication associated with T2D, comprising detecting at least one STR found to be in linkage disequilibrium with one of the SNPs of the present invention, wherein the presence of the STR in a sample of a subject (or patient) suffering from T2D indicates that said subject (or patient) is likely to develop the complication. Preferred examples of such complications include, but are not limited to, albuminuria and/or declining glomerular filtratrion myocardial infarction, and/or stroke. Methods for determining the presence of repeated sequences in a nucleic acid sample (for example, genomic DNA) are known in the art.

As such, in a related embodiment, the present invention provides a method for prognosticating type 2 diabetes-related complication in a subject comprising detecting single tandem repeats (STR) in a nucleic acid target sequence, wherein such target sequences are contained in at least one gene from the aforementioned gene set or a locus related thereto. The nucleotide sequences contained in the genes and/or a locus related thereto are obtainable from the GENEID and/or OMIM accession numbers.

It is understood that the SNP markers of this invention may be associated with other polymorphisms. This allows for tagging SNPs (tagSNPs), which comprise loci that can serve as proxies for many other SNPs. The use of tagSNPs greatly improves the power of association studies as only a subset of loci needs to be genotyped while maintaining the same information and power as if one had genotyped a larger number of SNPs.

By using the name of the aforementioned gene provided in Table G those skilled in the art will readily find the nucleotide sequences of a gene and the mRNAs encoded thereby as well as amino acid sequences the encoded polypeptides.

In certain methods described herein, an individual who is at risk for a T2D-related complication is an individual in whom one or more SNPs selected from Tables A-D are identified. In other embodiment also polymorphisms or haplotypes associated to SNPs of the tables may be used in risk assessment of a T2D-related complication. The significance associated with an allele or a haplotype is measured by an odds ratio. In a further embodiment, the significance is measured by a percentage. In one embodiment, a significant risk is measured as odds ratio of 0.9 or less or at least about 1.1, including by not limited to: 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 10%, including but not limited to about 10%, 25%, 30%, 35%, 40%, 45%, 50%, 55%, 60%, 65%, 70%, 75%, 80%, 85%, 90%, 95% and 99%. In a further embodiment, a significant increase in risk is at least about 50%. It is understood however, that identifying whether a risk is medically significant may also depend on a variety of factors such as family history of hypertension, history of gestational diabetes, previously identified glucose intolerance, obesity, hypertriglyceridemia, hypercholesterolemia, elevated LDL cholesterol, low HDL cholesterol, elevated blood pressure (BP), cigarette smoking, lack of physical activity, and inflammatory components as reflected by increased C-reactive protein levels or other inflammatory markers.

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize to its specific target. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Probes and primers may include modified bases as in polypeptide nucleic acids. Probes or primers typically comprise about 15 to 30 consecutive nucleotides present e.g. in human genome and they may further comprise a detectable label, e.g., radioisotope, fluorescent compound, enzyme, or enzyme co-factor. Probes and primers to a SNP marker disclosed in Tables A-D are available in the art or can easily be designed using the flanking nucleotide sequences assigned to a SNP rs ID and standard probe and primer design tools.

Primers and probes for SNP markers disclosed in Tables A-D can be used in risk assessment as well as molecular diagnostic methods and kits of this invention.

The invention comprises polyclonal and monoclonal antibodies that bind to a polypeptide encoded by a gene listed in table Gor comprising a SNP set forth in Tables A-D of the invention. The term “antibody” as used herein refers to immunoglobulin molecules or their immunologically active portions that specifically bind to an epitope (antigen, antigenic determinant) present in a polypeptide or a fragment thereof, but does not substantially bind other molecules in a sample, e.g., a biological sample, which contains the polypeptide. Examples of immunologically active portions of immunoglobulin molecules include F(ab) and F(ab′)₂ fragments which can be generated by treating the antibody with an enzyme such as pepsin. The term “monoclonal antibody” as used herein refers to a population of antibody molecules that are directed against a specific epitope and are produced either by a single clone of B cells or a single hybridoma cell line. Polyclonal and monoclonal antibodies can be prepared by various methods known in the art. Additionally, recombinant antibodies, such as chimeric and humanized monoclonal antibodies, comprising both human and non-human portions, can be produced by recombinant DNA techniques known in the art. Antibodies can be coupled to various enzymes, prosthetic groups, fluorescent materials, luminescent materials, bioluminescent materials, or radioactive materials to enhance detection.

In a related embodiment, the present invention also provides for the use of antisense oligonucleotides or silencing RNAs or similar methods which are capable of modulating the expression and/or levels of a product (i.e., mRNA or polypeptide) of a gene comprising a SNP set forth in Tables A-D. In a particularly preferred embodiment, the antisense molecules silencing RNAs or similar methods of the present invention are useful directed against the primary transcript (i.e., mRNA) of the genes listed in Table G or comprising a SNP set forth in Tables A-D. Techniques for the design and use of antisense molecules or silencing RNAs or similar methods, for example, in in vitro and/or in vivo applications, are known in the art.

“A T2D-related complication” in the context of this invention refers to glucose intolerance, insulin resistance, metabolic syndrome, obesity, a microvascular complication of T2D such as retinopathy, nephropathy or neuropathy, or a macrovascular complication such as coronary heart disease, cerebrovascular disease, congestive heart failure, claudication or other clinical manifestation of atherosclerosis or arteriosclerosis.

Preferred types of “T2D-related complications” include, but are not limited to, cardiovascular diseases, retinopathy, neuropathy, and/or nephropathy.

Particularly preferred “T2D-related complications” include, but are not limited to, myocardial infarction, stroke, albuminuria and/or declining glomerular filtration

An antibody specific for a polypeptide encoded by a gene identified in table G or containing a SNP listed in Tables A-D of the invention can be used to detect the polypeptide in a biological sample in order to evaluate the abundance and pattern of expression of the polypeptide. Antibodies can be used diagnostically to monitor protein levels in tissue such as blood as part of a test predicting the susceptibility to complications, such as, for example, myocardial infarction, stroke and/or albuminuria and/or declining glomerular filtration or as part of a clinical testing procedure, e.g., to, for example, determine the efficacy of a given treatment regimen. Highly purified antibodies (e.g. monoclonal humanized antibodies specific to a polypeptide encoded by an associated gene of the invention and/or polymorphic gene may be produced using GMP-compliant manufacturing processes known in the art. These “pharmaceutical grade” antibodies can be used in novel therapies modulating activity and/or function of a polypeptide encoded the associated gene(s) disclosed herein.

This invention provides information on genomic markers that can be used to develop methods, reagents and kits useful to predict diabetes complications. Development of such methods, reagents and kits relies on methods known to those skilled in the art, including without limitation allele specific PCR amplification or detection of such alleles, with or without prior amplification, with allele specific probes, and DNA sequencing. Information on genomic DNA sequences from which PCR primers, hybridization probes, and sequencing primers can designed can be found in public databases using the rs ID provided for each SNP in Tables A-D.

Diagnostic Methods and Test Kits

One major application of the current invention is diagnosing a susceptibility to T2D related complications. The risk assessment methods and test kits of this invention can be applied to any diabetic patient as a screening or predisposition test, although the methods and test kits are also be applied to prediabetic patients and other subjects, preferably those with high-risk individuals (who have e.g. family history of T2D, history of gestational diabetes, previous glucose intolerance, obesity or any combination of these). Diagnostic tests that define genetic factors contributing to T2D complications might be used together with or independent of the known clinical risk factors to define an individual's risk relative to the general population. Better means for identifying those individuals susceptible for a T2D-related complication should lead to better preventive and treatment regimens, including more aggressive management of the risk factors for a T2D-related complication such as obesity, lack of physical activity, hypercholesterolemia, elevated LDL cholesterol, low HDL cholesterol, elevated BP, cigarette smoking and inflammatory components as reflected by increased C-reactive protein levels or other inflammatory markers. Physicians may use the information on genetic risk factors to convince particular patients to adjust their life style e.g. to stop smoking, to reduce caloric intake or to increase exercise.

In one embodiment of the invention, diagnosis of a susceptibility to T2D related complication in a subject is made by detecting one or more SNP markers disclosed in Tables A-D of this invention in the subject's nucleic acid. The presence of assessed SNP markers or haplotypes in individual's genome indicates subject's increased risk for said T2D related complication. The invention also pertains to methods of diagnosing a susceptibility to said complication in an individual comprising detection of a haplotype in a genetic aspect that is more frequently present in an individual having a T2D complication (affected), compared to the frequency of its presence an individual not having a T2D complication (control), wherein the presence of the haplotype is indicative of a susceptibility to T2D-related complication. A haplotype may be associated with a reduced rather than increased risk of said complication, wherein the presence of the haplotype is indicative of a reduced risk of T2D-related complication.

In other embodiment of the invention, diagnosis of susceptibility to T2D-related complication, is done by detecting in the subject's nucleic acid one or more polymorphic sites which are in linkage disequilibrium with one or more SNP markers disclosed in Tables A-D of this invention. For a therapeutic purpose, the most useful polymorphic sites are those altering the biological activity of a polypeptide encoded by a T2D related complication gene set forth in Table G or comprising the SNPs listed in Tables A-D. Examples of such functional polymorphisms include, but are not limited to frame shifts; premature stop codons, amino acid changing polymorphisms and polymorphisms inducing abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other useful polymorphic sites are those affecting transcription of a gene set forth in Table G or comprising a SNP listed in Tables A-D or translation of its mRNA due to altered tissue specificity, due to altered transcription rate, due to altered response to physiological status, due to altered translation efficiency of the mRNA and due to altered stability of the mRNA. The presence of nucleotide sequence variants altering the polypeptide structure and/or expression in said associated genes in individual's nucleic acid is diagnostic for susceptibility to T2D-related complication but for a diagnostic purpose, the variant may also be included in uncharted areas of the genome.

In diagnostic assays determination of the nucleotides present in one or more SNP markers of this invention, as well as polymorphic sites associated therewith can be done by any method or technique which can accurately determine nucleotides present in a polymorphic site. Numerous suitable methods have been described in the art. These methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is obvious in the art the nucleotides present in a polymorphic site can be determined from either nucleic acid strand or from both strands.

In another embodiment of the invention, a susceptibility to a T2D-related complication is assessed from transcription products of one or more associated genes. Qualitative or quantitative alterations in transcription products can be assessed by a variety of methods described in the art, including e.g. hybridization methods, enzymatic cleavage assays, RT-PCR assays and microarrays. A test sample from an individual is collected and the alterations in the transcription of associated genes are assessed from the RNA molecules present in the sample. Altered transcription is diagnostic for a susceptibility to a T2D-related complication.

In another embodiment of the invention, diagnosis of a susceptibility to T2D-related complication is made by examining expression, abundance, biological activities, structures and/or functions of polypeptides encoded by one of the gene disclosed in Table Gor comprising the SNPs listed in Tables A-D. A test sample from an individual is assessed for the presence of alterations in the expression, biological activities, structures and/or functions of the polypeptides, or for the presence of a particular polypeptide variant (e.g., an isoform) encoded by a gene disclosed in table G or comprising the SNPs of Tables A-D. An alteration can be, for example, quantitative (an alteration in the quantity of the expressed polypeptide, i.e., the amount of polypeptide produced) or qualitative (an alteration in the structure and/or function of a polypeptide encoded by the polymorphic genes could be measured. Alterations in expression, abundance, biological activity, structure and/or function of polypeptides encoded by such polymorphic genes can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the polypeptide itself or its fragment or from substrates and reaction products of said polypeptide. A control sample is a sample that corresponds to the test sample (e.g., is from the same type of cells), and is from an individual who is not affected by a T2D complication. An alteration in the expression, abundance, biological activity, function or composition of a polypeptide encoded by a polymorphic gene of the invention in the test sample, as compared with the control sample, is indicative of a susceptibility to developing complications. In another embodiment, assessment of the splicing variant or isoform(s) of a polypeptide encoded by a polymorphic gene can be performed directly (e.g., by examining the polypeptide itself), or indirectly (e.g., by examining the mRNA encoding the polypeptide, such as through mRNA profiling).

Yet in another embodiment, a susceptibility to a T2D-related complication can be diagnosed by assessing the status and/or function of biological networks and/or metabolic pathways related to one or more polypeptides encoded by a Risk gene associated with T2D-related complication of this invention. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop said complication is evaluated by comparing observed status and/or function of biological networks and or metabolic pathways of a subject to the status and/or function of biological networks and or metabolic pathways of healthy controls.

Another major application of the current invention is diagnosis of a molecular subtype of a type II diabetic patient. Molecular diagnosis methods and kits of this invention can be applied to a person having type II diabetes. In one preferred embodiment, molecular subtype of T2D in an individual is determined to provide information of the molecular etiology of T2D. When the molecular etiology is known, better diagnosis and prognosis of T2D can be made and efficient and safe therapy for treating T2D-related complications in an individual can be selected on the basis of this genetic subtype. For example, a drug that is likely to be effective, for example, a blood glucose lowering agent, can be selected without trial and error. Physicians may use the information on genetic risk factors with or without known clinical risk factors to convince particular patients to adjust their life style and manage T2D risk factors and select intensified preventive and curative interventions for them. In other embodiment, biomarker information obtained from methods and kits of the present invention are used to select human subjects for clinical trials testing anti-diabetic drugs. The kits provided for diagnosing a molecular subtype of T2D in an individual comprise wholly or in part protocol and reagents for detecting one or more biomarkers and interpretation software for data analysis and T2D molecular subtype assessment.

The diagnostic assays and kits of the invention may further comprise a step of combining non-genetic information with the biomarker data to make risk assessment, diagnosis or prognosis of a T2D-related complication. Useful non-genetic information comprises, without limitations, are age, gender, smoking status, physical activity, waist-to-hip circumference ratio (cm/cm), the subject family history of T2D or obesity, history of gestational diabetes, previously identified glucose intolerance, obesity, hypertriglyceridemia, low HDL cholesterol, HT and particularly elevated BP and/or status of being hypertensive. The detection method of the invention may also further comprise a step determining blood, serum or plasma glucose, total cholesterol, HDL cholesterol, LDL cholesterol, triglyceride, apolipoprotein B and AI, fibrinogen, ferritin, transferrin receptor, C-reactive protein, serum or plasma insulin concentration.

The score that predicts the probability of developing a T2D-related complication may be calculated using art-known procedures including but not limited to logistic regression, support vector machines and neural networks. The results from the further steps of the method as described below render possible a step of calculating the probability of developing such T2D-related complication using a logistic regression equation. Alternative statistical models include, but are not limited to, Cox's proportional hazards' model, other iterative models and neural networking models.

Diagnostic test kits (e.g. reagent kits) of this invention comprise reagents, materials and protocols for assessing one or more biomarkers, and instructions and software for comparing the biomarker data from a subject to biomarker data from healthy and diseased people to make risk assessment, diagnosis or prognosis of a T2D related complication and optimized therapeutic suggestions. Useful reagents and materials for kits include, but are not limited to PCR primers, hybridization probes and primers as described herein (e.g., labeled probes or primers), allele-specific oligonucleotides, reagents for genotyping SNP markers, reagents for detection of labeled molecules, restriction enzymes (e.g., for RFLP analysis), DNA polymerases, RNA polymerases, DNA ligases, marker enzymes, antibodies which bind to altered or to non-altered (native) a polypeptide, means for amplification of nucleic acids fragments from one or more SNPs selected from Tables A-D, means for analyzing the nucleic acid sequence of one or more T2D-complication related SNPs, or means for analyzing the sequence of one or more amino acid residues of polypeptides encoded by genes comprising such SNPs, etc. In one embodiment, a kit for diagnosing susceptibility to a T2D-related complication comprises primers and reagents for detecting the nucleotides present in one or more SNP markers selected from the Tables A-D in individual's nucleic acid.

Methods of Therapy

The present invention discloses novel methods for the prevention and treatment of a T2D-related complication. In particular, the invention relates to methods of treatment of T2D-related complications. The term, “treatment” as used herein, refers not only to ameliorating symptoms associated with the disease, but also preventing or delaying the onset of the complication, and also lessening the severity or frequency of symptoms of the disease, preventing or delaying the occurrence of a second episode of the disease or condition; and/or also lessening the severity or frequency of symptoms of the disease or condition.

The present invention encompasses methods of treatment (prophylactic and/or therapeutic) for a T2D-related complication using a therapeutic agent. A “therapeutic agent” is an agent that alters (e.g., enhances or inhibits) enzymatic activity or function of a risk gene such as those disclosed in table G or containing the SNPs of Tables A-D and/or expression of polymorphisms disclosed in Tables A-D and/or the specific metabolic or other biologically related pathway implicating those genes. The modes of useful therapeutic agents are further disclosed.

Representative therapeutic agents of the invention comprise the following: (a) nucleic acids, fragments, variants or derivatives of the genes, nucleic acids, or an active fragment or a derivative thereof and nucleic acids modifying the expression of said genes (e.g. antisense polynucleotides, catalytically active polynucleotides (e.g. ribozymes and DNAzymes), molecules inducing RNA interference (RNAi) and micro RNA), and vectors comprising said nucleic acids; (b) polypeptides, active fragments, variants or derivatives thereof, binding agents of polypeptides; peptidomimetics; fusion proteins or prodrugs thereof, antibodies; (c) metabolites of the gene products; (d) small molecules and compounds that alter (e.g., inhibit or antagonize) a risk gene expression, activity and/or function of a polypeptide encoded by said genes and; (e) small molecules and compounds that alter (e.g. induce, agonize or modulate) the expression or activity of said genes.

The nucleic acid sequences of T2D-related complication associated risk genes such as those disclosed in Table G and/or polymorphisms disclosed in Tables A-D of this application are publicly available and can be used to design and develop therapeutic nucleic acid molecules and recombinant DNA molecules for the prevention and treatment of T2D or a T2D related condition. For example antisense nucleic acid molecules targeted to a polymorphism in Tables A-D can be designed using tools and the nucleotide sequence of the gene available in the art and constructed using chemical synthesis and/or enzymatic ligation reactions using procedures known in the art. Antisense nucleic acid molecule can be chemically synthesized using naturally occurring nucleotides or modified nucleotides designed to increase the biological stability of the molecules or to increase the physical stability of the duplex formed between the antisense oligonucleotide and sense nucleic acids, e.g., phosphorothioate derivatives and acridine substituted nucleotides can be used.

Alternatively, the antisense nucleic acid molecule can be produced biologically using an expression vector into which a nucleic acid molecule encoding a T2D-related complication risk gene, a fragment or a variant thereof has been cloned in antisense orientation (i.e., RNA transcribed from the expression vector will be complementary to the transcribed RNA of a Risk gene associated with T2D-related complication of interest).

More than one T2D-related complication therapeutic agent can be used concurrently, if desired. The therapy is designed to affect 1) expression of a T2D-related complication gene in a sense of activation, inhibition or modulation, 2) abundance, stability, biological activity and/or function of a risk gene associated with T2D-related complication-encoded ribonucleic acid or polypeptide, or 3) biological activity and/or function of a T2D-related complication gene related signaling or metabolic pathway. Upregulation or increasing expression of a risk gene associated with T2D-related complication or a particular variant of a risk gene could interfere with or compensate for the expression or activity of a defective gene or variant; downregulation or decreasing expression or availability of a native risk gene or a particular splicing variant of a susceptibility gene associated with T2D-related complication could minimize the expression or activity of a defective gene or the particular variant and thereby minimize the impact of the defective gene or the particular variant.

The T2D and T2D-related complication therapeutic agent(s) are administered in a therapeutically effective amount that can be determined using established clinical methods and assays. The precise dose to be employed in the formulation will also depend on the route of administration, and the seriousness of the disease or disorder, and should be decided according to the judgment of a practitioner. Effective doses may be extrapolated from dose-response curves derived from in vitro or animal model test systems.

In one embodiment, a nucleic acid encoding a T2D-related complication polypeptide, fragment, variant or derivative thereof, either by itself or included within a vector, can be introduced into cells of an individual affected by T2D or a T2D related condition using variety of experimental methods described in the art, so that the treated cells start to produce native T2D-related complication susceptibility polypeptide. Thus, cells which, in nature, lack of a native Risk gene associated with T2D-related complication expression and activity, or have abnormal Risk gene associated with T2D-related complication expression and activity, can be engineered to express a T2D-related complication polypeptide or an active fragment or a different variant of said T2D-related complication susceptibility polypeptide. Genetic engineering of cells may be done either “ex vivo” (i.e. suitable cells are isolated and purified from a patient and re-infused back to the patient after genetic engineering) or “in vivo” (i.e. genetic engineering is done directly to a tissue of a patient using a vehicle). Alternatively, in another embodiment of the invention, a nucleic acid (e.g. a polynucleotide) which specifically hybridizes to the mRNA and/or genomic DNA of a T2D-related complication gene is administered in a pharmaceutical composition to the target cells or said nucleic acid is generated “in vivo”. The antisense nucleic acid that specifically hybridizes to the mRNA and/or DNA inhibits expression of the T2D-related complication polypeptide, e.g., by inhibiting translation and/or transcription. Binding of the antisense nucleic acid can be due to conventional base pairing, or, for example, in the case of binding to DNA duplexes, through specific interaction in the major groove of the double helix. In a preferred embodiment nucleic acid therapeutic agents of the invention are delivered into cells that express one or more T2D-related complication risk genes. A number of methods including, but not limited to, the methods known in the art can be used for delivering a nucleic acid to said cells. For example, a vector can be introduced in vivo such that it is taken up by a cell and directs the transcription of a RNA molecule, which induces RNA interference in the cell. Such a vector can remain episomal or become chromosomally integrated, and as long as it can be transcribed to produce the desired RNA molecules it will modify the expression of a Risk gene associated with T2D-related complication. Such vectors can be constructed by various recombinant DNA technology methods standard in the art.

The expression of an endogenous Risk gene associated with T2D-related complication can be reduced by inactivating completely (“knocking out”) or partially (“knocking down”) a T2D-related complication gene using targeted homologous recombination methods described in the art. Alternatively, expression of a functional, non-mutant T2D-related complication can be increased using a similar method: targeted homologous recombination can be used to replace a non-functional Risk gene associated with T2D-related complication with a functional form of the said gene in a cell. In another embodiment of the invention, other T2D therapeutic agents as described herein can also be used in the treatment or prevention of T2D or a related condition. The therapeutic agents can be delivered in a pharmaceutical composition; they can be administered systemically, or can be targeted to a particular tissue. The therapeutic agents can be produced by a variety of means, including chemical synthesis, cell culture and recombinant techniques (e.g. with transgenic cells and animals). Therapeutic agents can be isolated and purified to fulfill pharmaceutical requirements using standard methods described in the art. A combination of any of the above methods of treatment (e.g., administration of non-mutant T2D-related complication susceptibility polypeptide in conjunction with RNA molecules inducing RNA interference targeted to the mutant T2D-related complication susceptibility mRNA) can also be used.

In the case of pharmaceutical therapy the invention comprises compounds which affect 1) expression of a T2D-related complication gene in a sense of activation, inhibition or modulation, 2) abundance, stability, biological activity and/or function of a Risk gene associated with T2D-related complication-encoded ribonucleic acid or polypeptide, or 3) biological activity and/or function of a T2D-related complication gene or metabolic pathway encoded by T2D complication-associated risk genes such as those disclosed in Table G and/or linked to polymorphisms disclosed in Tables A-D of this application. The treatment may also exert its effects as specified above on one or several genes selected from the T2D complication-associated risk genes such as those disclosed in Table G and/or associated with the polymorphisms disclosed in Tables A-D of this application.

Furthermore, a disclosed method or a test based on biomarkers specific for T2D-related complication susceptibility gene is useful in selection, modification or optimalization of therapeutic modalities for T2D-patients. For example if the less frequent, i.e. the minor, assumable mutated allele in the T2D-related complication susceptibility gene is risk-reducing, and if said mutation is a gene function reducing mutation, one can deduce that the gene function and/or activity would increase the risk of T2D complication. On that basis, drugs and other therapies such as gene therapies that reduce or inhibit the function or activity of the T2D-related complication susceptibility gene or the encoded protein would reduce the risk of the said T2D-related complication and could be used to both prevent and treat the said T2D-related complication in subjects having said mutated allele.

In another embodiment of the invention a T2D or T2D-related complication therapeutic agent comprises a known therapeutic agent related to a T2D-related complication associated gene listed in tables G or associated with the SNP having the RefSNP ID Nos. in Tables A-D of this invention but which is not used to treat T2D or a T2D-related complication. Such compounds and therapeutic agents are useful for developing new therapies for T2D or a T2D-related complication as they most likely affect 1) expression of a T2D-related complication gene in a sense of activation, inhibition or modulation, 2) abundance, stability, biological activity and/or function of a Risk gene associated with T2D-related complication-encoded ribonucleic acid or polypeptide, or 3) biological activity and/or function of a T2D-related complication gene related signaling or metabolic pathway. These agents may be used alone or in combination with other treatments and agents used for prevention or treatment of T2D or a T2D-related condition.

In one embodiment of the invention, therapeutic agents or compounds currently utilized for the treatment of T2D and T2D-related complications are combined with one or more known therapeutic agents used to treat T2D comprising I. oral antidiabetics including biguanid derivatives such as 1) metformin, 2) buformin, insulin secretagogues such as 1) sulphonylurea derivatives such as tolbutamide, glibenclamide, gliclazide, glipizide, glimepiride, gliquidone; 2) meglitinides such as repaglinide, nateglinide; 3) alpha-glucosidase inhibitors such as acarbose, miglitol; 4) thiazolidinediones such as rosiglitazone and pioglitazone; 5) other defined by World Health Organisation—The Anatomical Therapeutic Chemical (ATC) classification system; II. insulin such as i) insulin glargine, ii) insulin aspart, iii) insulin lispro, iv) insulin glulisine; v) insulin detemir; and agents known do decrease and or prevent diabetes related complication, such as high blood pressure, i) converting enzyme inhibitors, ii) angiotensin receptor blockers, iii) direct renin inhibitors, iv) endothelin antagonists, v) diuretics, vi) beta blockers, vii) alpha blockers, viii) inhibitors of phosphodiesterase 5a and the combinations thereof.

Pharmaceutical Compositions

The present invention also pertains to pharmaceutical compositions comprising agents described herein, particularly polynucleotides, polypeptides and any fractions, variants or derivatives of T2D-related complication genes, and/or agents that alter (e.g., enhance or inhibit) expression of a risk gene or genes, or activity of one or more polypeptides encoded by associated genes as described herein. For instance, an agent that alters expression of a risk gene or activity of one or more polypeptides encoded thereby.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents. The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration. For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of a T2D-related complication, and should be decided according to the judgment of a practitioner and each patient's circumstances. Effective doses may be extrapolated from dose-response curves derived from in vitro or animal model test systems.

Functional Foods

By definition “functional foods” or “nutraceuticals” are foods or dietary components or food ingredients that may provide a health benefit beyond basic nutrition. Functional foods are regulated by authorities (e.g. by the FDA in US) according to their intended use and the nature of claims made on the package. Functional foods can be produced by various methods and processes known in the art including, but not limited to synthesis (chemical or microbial), extraction from a biological material, mixing functional ingredient or component to a regular food product, fermentation or using a biotechnological process. A functional food may exert its effects directly in the human body or it may function e.g. through human intestinal bacterial flora.

The associated genes disclosed in Table G or associated with the SNPs of Tables A-D of this invention can be used as molecular targets towards which functional foods claiming health benefit in a T2D related complication can be developed. For example a functional food may compensate reduced biological activity of a polypeptide encoded by a gene associated with the SNPs of Tables A-D when the risk gene is defective or is not expressed properly in a subject. A functional food may also inhibit the expression and/or biological activity of a gene or polypeptide of the invention promoting the development of a T2D related complication. In another embodiment a functional food may increase the expression and/or biological activity of a gene or polypeptide protecting an individual from the development of a T2D related complication due to reduced expression and protein production.

Aspects of the instant invention include, but are not limited to:

Aspect 1. A method for predicting increased risk of developing a complication which is myocardial infarction, stroke, or kidney complication in a subject affected by T2D, comprising detecting, in a sample obtained from said subject, at least one genetic feature which is single nucleotide polymorphism (SNP) or short tandem repeat (STR), wherein the detection of said genetic feature in said subject correlates with said increased risk of developing at least one of said complication.
Aspect 2. The method according to aspect 1, wherein said SNP comprises a polymorphism of a gene or a locus linked thereto.
Aspect 3. The method according to aspect 1, wherein said genetic feature is
(a) a SNP having the RefSNPID Nos. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544;
(b) at least one SNP which is in linkage disequilibrium with at least one SNP of (a); or
(c) short tandem repeat (STR) that is in linkage disequilibrium with at least one SNP of (a).
Aspect 4. The method according to aspect 3, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264, at least 265, at least 266, at least 267, at least 268, at least 269, at least 270, at least 271, at least 272, at least 273, at least 274, at least 275, at least 276, at least 277, at least 278, at least 279, at least 280, at least 281, at least 282, at least 283, at least 284, at least 285, at least 286, at least 287, at least 288, at least 289, at least 290, at least 291, at least 292, at least 293, at least 294, at least 295, at least 296, at least 297, at least 298, at least 299, at least 300, at least 301, at least 302, at least 303, at least 304, at least 305, at least 306, at least 307, at least 308, at least 309, at least 310, at least 311, at least 312, at least 313, at least 314, at least 315, at least 316, at least 317, at least 318, at least 319, at least 320, at least 321, at least 322, at least 323, at least 324, at least 325, at least 326, at least 327, at least 328, at least 329, at least 330, at least 331, at least 332, at least 333, at least 334, at least 335, at least 336, at least 337, at least 338, at least 339, at least 340, at least 341, at least 342, at least 343, at least 344, at least 345, at least 346, at least 347, at least 348, at least 349, at least 350, at least 351, at least 352, at least 353, at least 354, at least 355, at least 356, at least 357, at least 358, at least 359, at least 360, at least 361, at least 362, at least 363, at least 364, at least 365, at least 366, at least 367, at least 368, at least 369, at least 370, at least 371, at least 372, at least 373, at least 374, at least 375, at least 376, at least 377, at least 378, at least 379, at least 380, at least 381, at least 382, at least 383, at least 384, at least 385, at least 386, at least 387, at least 388, at least 389, at least 390, at least 391, at least 392, at least 393, at least 394, at least 395, at least 396, at least 397, at least 398, at least 399, at least 400, at least 401, at least 402, at least 403, at least 404, at least 405, at least 406, at least 407, at least 408, at least 409, at least 410, at least 411, at least 412, at least 413, at least 414, at least 415, at least 416, at least 417, at least 418, at least 419, at least 420, at least 421, at least 422, at least 423, at least 424, at least 425, at least 426, at least 427, at least 428, at least 429, at least 430, at least 431, at least 432, at least 433, at least 434, at least 435, at least 436, at least 437, at least 438, at least 439, at least 440, at least 441, at least 442, at least 443, at least 444, at least 445, or more of said SNPs.
Aspect 5. The method according to aspect 1, wherein said complication is stroke and said method comprises detection of at least one SNP having the RefSNP ID No. rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, and/or rs10189134.
Aspect 6. The method according to aspect 5, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, or more of the aforementioned SNPs.
Aspect 7. The method according to aspect 1, wherein said complication is MI and said method comprises detection of at least one SNP having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, and/or rs17191463.
Aspect 8. The method according to aspect 7, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, or more of the SNPs.
Aspect 9. The method according to aspect 1, wherein said complication is kidney-related complication and said method comprises detection of at least one SNP having the RefSNP ID No. rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs 10922204, rs2066926, rs3917768, rs2851870, rs2444242, rs 12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs7765427, rs963264, rs1258323, rs28490018, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs6814800, rs3909788, rs7690478, rs6810410, rs7549770, rs7935913, rs1161262, rs4497678, rs10155009, rs4258086, rs11927384, rs4269885, rs16825798, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs11848645, rs7203172, rs2171304, rs6585304, rs7967892, rs7501838, rs3883317, rs12973797, rs3809485, rs6589065, rs765, rs7535769, rs11688740, rs10514925, rs13333226, rs4702266, rs980907, rs17600042, rs9540649, rs634585, rs1327210, rs17680407, rs17680905, rs1913695, rs191606, rs11951359, rs10071774, rs10471462, rs617595, rs7195806, rs4940275, rs16880395, rs7748736, rs9293123, rs9502593, rs17078168, rs9287229, rs7243021, rs2936535, rs11183154, rs2324872, rs1218661, rs12511845, rs11752805, rs11707973, rs2740485, rs4936167, rs4572098, rs243839, rs2962394, rs7334517, rs130250, rs4378283, rs1364406, rs10153433, rs10248275, and/or rs1519729.
Aspect 10. The method according to aspect 9, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, or more of the aforementioned SNPs.
Aspect 11. The method according to aspect 1, comprising detecting a SNP having the RefSNP ID No. rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544.
Aspect 12. The method according to aspect 11, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, or more of the aforementioned SNPs.
Aspect 13. The method according to aspect 6, comprising detecting a combination of SNPs, wherein said combination comprises at least 50 or more of the aforementioned SNPs.
Aspect 14. The method according to aspect 8, comprising detecting a combination of SNPs, wherein said combination comprises at least 50 or more of the aforementioned SNPs.
Aspect 15. The method according to aspect 10, comprising detecting a combination of SNPs, wherein said combination comprises at least 50 or more of the aforementioned SNPs.
Aspect 16. The method according to aspect 12, comprising detecting a combination of SNPs, wherein said combination comprises at least 50 or more of the aforementioned SNPs.
Aspect 17. The method according to aspect 3, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.
Aspect 18. The method according to aspect 5, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.
Aspect 19. The method according to aspect 7, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.
Aspect 20. The method according to aspect 9, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.
Aspect 21. The method according to aspect 11, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.
Aspect 22. A kit for predicting a complication which is myocardial infarction, stroke or kidney complication in a subject affected by type-2 diabetes (T2D) comprising in one or more packages
(a) an oligonucleotide that specifically hybridizes to a SNP having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) an oligonucleotide which is the complement of (a);
and one or more reagents for the detection of said oligonucleotide.
Aspect 23. The kit according to aspect 22, comprising one or more reagents for polymerase chain reaction (PCR).
Aspect 24. The kit of aspect 22, further comprising an RNase.
Aspect 25. The kit of aspect 22, which further comprises one or more reagents for isolation of cells from a sample.
Aspect 26. The kit of aspect 22, further comprising a DNAse inhibitor.
Aspect 27. The kit of aspect 22, further comprising reagents for sequencing in one or more packages.
Aspect 28. The kit of aspect 22, further comprising a cDNA microarray plate in one or more packages.
Aspect 29. The kit of aspect 22, which comprises a combination of oligonucleotides which hybridize to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264, at least 265, at least 266, at least 267, at least 268, at least 269, at least 270, at least 271, at least 272, at least 273, at least 274, at least 275, at least 276, at least 277, at least 278, at least 279, at least 280, at least 281, at least 282, at least 283, at least 284, at least 285, at least 286, at least 287, at least 288, at least 289, at least 290, at least 291, at least 292, at least 293, at least 294, at least 295, at least 296, at least 297, at least 298, at least 299, at least 300, at least 301, at least 302, at least 303, at least 304, at least 305, at least 306, at least 307, at least 308, at least 309, at least 310, at least 311, at least 312, at least 313, at least 314, at least 315, at least 316, at least 317, at least 318, at least 319, at least 320, at least 321, at least 322, at least 323, at least 324, at least 325, at least 326, at least 327, at least 328, at least 329, at least 330, at least 331, at least 332, at least 333, at least 334, at least 335, at least 336, at least 337, at least 338, at least 339, at least 340, at least 341, at least 342, at least 343, at least 344, at least 345, at least 346, at least 347, at least 348, at least 349, at least 350, at least 351, at least 352, at least 353, at least 354, at least 355, at least 356, at least 357, at least 358, at least 359, at least 360, at least 361, at least 362, at least 363, at least 364, at least 365, at least 366, at least 367, at least 368, at least 369, at least 370, at least 371, at least 372, at least 373, at least 374, at least 375, at least 376, at least 377, at least 378, at least 379, at least 380, at least 381, at least 382, at least 383, at least 384, at least 385, at least 386, at least 387, at least 388, at least 389, at least 390, at least 391, at least 392, at least 393, at least 394, at least 395, at least 396, at least 397, at least 398, at least 399, at least 400, at least 401, at least 402, at least 403, at least 404, at least 405, at least 406, at least 407, at least 408, at least 409, at least 410, at least 411, at least 412, at least 413, at least 414, at least 415, at least 416, at least 417, at least 418, at least 419, at least 420, at least 421, at least 422, at least 423, at least 424, at least 425, at least 426, at least 427, at least 428, at least 429, at least 430, at least 431, at least 432, at least 433, at least 434, at least 435, at least 436, at least 437, at least 438, at least 439, at least 440, at least 441, at least 442, at least 443, at least 444, at least 445, or at least 446 of the SNPs of claim 21.
Aspect 30. The kit of aspect 22, wherein the oligonucleotide is
(a) an oligonucleotide comprising at least 80% sequence identity to the oligonucleotide having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) an oligonucleotide which is complimentary to the oligonucleotide of (a).
Aspect 31. The kit of aspect 30, wherein the oligonucleotide is
(c) an oligonucleotide comprising at least 90% sequence identity to the oligonucleotide having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(d) an oligonucleotide which is complimentary to the oligonucleotide of (a).
Aspect 32. The kit of aspect 30, wherein the oligonucleotide is
(e) an oligonucleotide comprising at least 95% sequence identity to the oligonucleotide having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(f) an oligonucleotide which is complimentary to the oligonucleotide of (a).
Aspect 33. The kit of aspect 30, wherein the oligonucleotide is
(g) an oligonucleotide comprising at least 100% sequence identity to the oligonucleotide having the RefSNP ID listed aspect 30; or
(h) an oligonucleotide which is complimentary to the oligonucleotide of (a).
Aspect 34. The kit of aspect 22, which further comprises a control oligonucleotide that hybridizes to the wild-type allele.
Aspect 35. Use of a kit according to aspect 22 for predicting a complication which is myocardial infarction, stroke, albuminuria or kidney complication in a subject affected by type-2 diabetes (T2D).
Aspect 36. A method for preventing, treating or reducing the risk of T2D or a T2D related complication which is myocardial infarction, stroke, albuminuria or declining kidney complication in a subject in need thereof, comprising administrating to said subject a therapeutic agent which
(a) alters the enzymatic activity or levels of a polypeptide encoded by at least one risk gene listed in Table G or one gene associated with a SNP having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) alters the expression of at least one polymorphism disclosed in (a); or
(c) alters the specific metabolic or other biologically related pathway implicating the risk gene of (a).
Aspect 37. The method according to aspect 36, wherein the therapeutic agent alters the expression of at least one polymorphism having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544.
Aspect 38. The method according to aspect 37, wherein the therapeutic agent is an antisense oligonucleotide or an siRNA.
Aspect 39. The method according to aspect 36, wherein the therapeutic agent alters the levels or enzymatic activity of a polypeptide encoded by at least one risk gene listed in Table G.
Aspect 40. The method according to aspect 39, wherein the therapeutic agent is an antibody.
Aspect 41. A method for identifying a subject for preventive therapeutic action, comprising detecting, in a sample obtained from said subject, at least one genetic feature which is
(a) at least one single nucleotide polymorphism (SNP) having the RefSNPID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544;
(b) at least one SNP which is in linkage disequilibrium with at least one SNP of (a); or
(c) short tandem repeat (STR) that is in linkage disequilibrium with at least one SNP of (a),
wherein the detection of said genetic feature in said subject correlates with the eligibility of said subject for said preventive therapeutic action.
Aspect 42. A method for predicting increased risk of developing a complication which is myocardial infarction, stroke, or kidney complication in a subject affected by T2D, comprising detecting, in a sample obtained from said subject, at least one genetic feature which is single nucleotide polymorphism (SNP) or short tandem repeat (STR) of at least one gene which is listed in Table G, wherein the detection of said genetic feature in said subject correlates with said increased risk of developing at least one of said complication.
Aspect 43. The method according to Aspect 42, comprising detecting a combination of SNPs in The method according to aspect 3, comprising detecting a combination of SNPs, wherein said combination comprises SNPs of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, or more of said genes of Table G.

The entire disclosures of all applications, patents and publications, cited above and below, are hereby incorporated by reference.

Methods

Over the last year several studies have reported significant associations of single nucleotide polymorphisms (SNP) using Genome-wide Associations Studies (GWAS) in several areas, including myocardial infarction and T2D. Here we analyzed the micro- and macro-vascular complications in type 2 diabetics at the entry of ADVANCE study.

Total genomic DNA was extracted from human blood with FlexiGene DNA kit from Qiagen and dissolved in sterile TE buffer. DNA collection was preserved at a standard concentration of 1 μg/μl in a cold room. ADVANCE patients (n=2313) of Caucasian origin (Pritchard et al. Inference of population structure using multilocus genotype data. Genetics, 55:945-59, 2000) having several complications of T2D were compared to controls T2D patients without such complications, recruited for older age or long T2D duration. All patients' DNA was genotyped by Affymetrix GeneChip® SNP arrays 5.0 or 6.0. This assay is comprised of 1 array and two assay kits. The array is designed to detect over 906 000 single nucleotide polymorphism (SNPs) on the human genome plus 946 000 copy number variants. Genome-wide human SNP Nsp/Sty assay kit was used (Purcell et al. A toolset for whole-genome association and population-based linkage analysis. American Journal of Human Genetics, 81, 2007). For SNP mapping, genomic DNA was diluted with reduced EDTA TE buffer at a concentration of 50 ng/μl. The assay required 500 ng of genomic DNA. The principle of this assay is based on a strategy that reduces the complexity of the human genome by digesting DNA with NspI and StyI restriction enzymes (RE), ligating RE specific sequences at the end of DNA fragments and preferentially amplifying 250-2000 base pairs amplicons of RE fragments by a single PCR primer. 50 μg of the purified PCR products are then submitted to fragmentation with DNase Ito a size of 20-100 base pairs, end-labelled and injected into SNP arrays 5.0 or 6.0 to be hybridised for 16 hours at 50° C. The arrays are washed and stained in the Affymetrix F-450 fluidics station in a three stage automated process consisting of a streptavidin-phycoerythin (SAPE) stain followed by a biotinylated anti-streptavidin antibody amplification and final stain with streptavidin-phycoerythin. Following staining, the arrays are filled with buffer and scanned with the Affymetrix GeneChip Scanner 3000 7G.

The automation of the fluidics station and the scanner is supported by the Affymetrix genotyping command console (AGCC) as well as the samples and experiment registration, the image acquisition and the image files exportation to genotyping console for further analysis. Genotyping console is a software application that allows the analysis and the evaluation of the data from the image of the array and generates genotype calls. It creates reports of the analysis and the data and allows the exportation of those reports and data to other software applications for biostatistical analysis.

The genome wide association study (GWAS) was done with PLINK v1.07 PLINK [Purcell S, Neale B, Todd-Brown K, Thomas L, Ferreira M A R, Bender D, Maller J, Sklar P, de Bakker P I W, Daly M J & Sham P C. (2007). PLINK: a toolset for whole-genome association and population-based linkage analysis. American Journal of Human Genetics, 81.] to generate the lists of SNPs associated to micro and micro/macrovascular complications, stroke, myocardial infarction and kidney complications. Analyses were performed using the minor allele for each SNP, the minor allele being defined as the allele that was found the least frequent in the population under study. With some exception, the threshold of p-value of SNPs associated to our different outcomes was 10⁻³. The list of SNPs generated by GWAS was submitted to the NetAffx™ Analysis Center (available at world-wide-web URL at affymetrix.com/analysis/index.affx) to generate gene lists.

Gene lists were then submitted to IPA software (Ingenuity Systems, Redwood City, Calif., USA) for search against the Ingenuity® Knowledge Base, a repository of biological interactions and functional annotations created from millions of individually modeled relationships between proteins, genes, complexes, cells, tissues, metabolites, drugs, and diseases. These modeled relationships include rich contextual details, links to the original article, and are manually reviewed for accuracy. The Ingenuity Knowledge Base is used as a starting point for exploration and a functional bridge between novel discovery and known biology. It provides with a powerful resource for searching relevant and substantiated knowledge from the literature, and for interpreting experimental results in the context of larger biological systems for greater confidence with research decisions. After being searched against the Ingenuity Knowledge Base, each gene of the gene lists was checked for its relationship with diseases classes. Among those, 166 genes were common to 5 significantly enriched disease classes (endocrine system (T1D and T2D), cardiovascular, immunological, neurological and inflammatory diseases) and constituted a major network comprising several gene families of interest. These genes are listed in Table G. SNPs associated to micro and micro/macrovascular complications, stroke, myocardial infarction and kidney complications and located within or in the close vicinity of these genes were retained as the most significant and are listed in Tables A, B, C, D, E.

### BRIEF DESCRIPTION OF THE DRAWINGS AND TABLES

Various features and attendant advantages of the present invention will be more fully appreciated as the same becomes better understood when considered in conjunction with the accompanying drawings, in which like reference characters designate the same or similar parts throughout the several views, and wherein:

Table A: Lists SNP biomarkers that are predictive of risk of developing stroke in T2D subjects. Structural description of these SNPs, identification of the minor allele and the OR associated with this minor allele is provided.

Table B: Lists SNP biomarkers that are predictive of risk of developing myocardial infarction in T2D subjects. Structural description of these SNPs, identification of the minor allele and the OR associated with this minor allele is provided.

Table C. Lists SNP biomarkers that are predictive of risk of developing kidney complications (e.g., declining filtration, nephropathy, albuminuria, etc.) in T2D subjects. Structural description of these SNPs, identification of the minor allele and the OR associated with this minor allele is provided. Also are provided the specific kidney complication each SNP is associated with: albuminuria (Albumin/creatinine ratio ≧30 microgram/milligram) or; stratified as microalbuminuria (Albumin/creatinine ratio ≧30 microgram/milligram and <300 ug/mg) or macroalbuminuria (Albumin/creatinine ratio ≧300 microgram/milligram) and low creatinine clearance/low glomerular filtration (Cockcroft-Gault estimated creatinine clearance <60 ml/min/1.73 m2)

Table D. Lists SNP biomarkers that are predictive of risk of developing complications associated with type-2-diabetes. Complications include, for example, a combination of stroke, myocardial infarction, and/or kidney complications. Structural description of these SNPs, identification of the minor allele and the OR associated with this minor allele is provided.

Table E. Global list of SNP biomarkers of the instant invention.

Table F. Lists SNP biomarkers that are associated with T2D risk genes.

Table G: List of genes associated with SNPs of the present invention.

Without further elaboration, it is believed that one skilled in the art can, using the preceding description, utilize the following invention to its fullest extent. The following specific preferred embodiments are, therefore, to be construed as merely illustrative, and not limitative of the remainder of the disclosure in any way whatsoever.

In the forgoing and in the following examples, all temperatures are set forth uncorrected in degrees Celsius and, all parts and percentages are by volume, unless otherwise indicated.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions. All publications and patents cited above are incorporated herein by reference.

TABLE A

| | chromo- | | | Minor | | pheno- |
|---|---|---|---|---|---|---|
| rs_ID | some | marshfield | Flanking Sequence | Allele | OR | type |
| rs10131139 | 14 | 132.278260407308 | CATTTTGTTTCATTAA[C/T]CCAAGACTTAATTTGG | C | 0.6405 | stroke |
| rs10189134 | 2 | 182.81317432424 | ATAGGGAGAATGCTCA[C/T]TAATGGTGAACATCTC | C | 1.048 | stroke |
| rs10235865 | 7 | 98.3883438646555 | TATCATAGCACTTATA[C/G]AGTGTTGTGCCATTGT | C | 1.399 | stroke |
| rs1029969 | 16 | 12.0300990630897 | TTAAGTCACTTGCCCA[A/G]GATTATTCAGGACGTA | G | 0.8106 | stroke |
| rs10487028 | 7 | 98.3940381675499 | TGTCAGATCTGCGGAA[A/C]GATGAGTAGAATGGGT | A | 1.403 | stroke |
| rs10487029 | 7 | 98.3947008764778 | GCTAAGGGCCTTCAAA[A/G]TTTCTATTGCTTTACA | G | 1.397 | stroke |
| rs1061577 | 10 | 53.5494142760699 | AAATGAAAACACACAA[A/G]TTGAAAATGCACAGAA | G | 1.657 | stroke |
| rs11151820 | 18 | 107.888183050428 | CTTGAAATTTCCTCTA[G/T]GATAGCTTCTCTTTGG | G | 1.498 | stroke |
| rs11237675 | 11 | 82.1029607054341 | CCCCTGGCATAGTAGT[C/T]AAAGGCTGTAGGAGCC | C | 1.39 | stroke |
| rs11752725 | 6 | 153.688064175937 | GTTGTCTTAGAAGCCA[C/T]GGGAAGCAAACGTATG | T | 0.6724 | stroke |
| rs12454952 | 18 | 107.888341961581 | GATTTTCTGCCAAGAC[A/G]ATAAGAAGAGTACTTT | A | 1.459 | stroke |
| rs12539689 | 7 | 12.5482225425386 | CTAGTGCATCATTGAA[C/G]GAAAGGAATTTCACAC | G | 1.769 | stroke |
| rs13166738 | 5 | 131.69531682283 | TAAGAGAATGAATGTC[C/T]AGCCAACCACTGGATT | T | 0.4873 | stroke |
| rs13253981 | 8 | 60.3028784275937 | GACACATTTGCTAAAA[C/T]TATACAATCACAGCAA | T | 1.291 | stroke |
| rs1368761 | 6 | 98.6157181271585 | AATATTTTTCTCTACC[C/G]GAATCCATCAGCTACC | G | 2.204 | stroke |
| rs1498600 | 5 | 68.7170901128291 | GGTGCCAAGCTAGTCA[A/G]TCAGTGGCAAAGCTCA | A | 1.375 | stroke |
| rs1526591 | 3 | 71.7409225371831 | GGTTTGTGTGTACTTA[G/T]CTAATTTTTAAAGAAG | G | 1.679 | stroke |
| rs1550617 | 11 | 33.326339526741 | AGGTGGACCTTCAGAC[A/G]ATCTTAAAGTGTCAGT | A | 1.452 | stroke |
| rs1602 | 3 | 224.323354760653 | AGTGAGACATGGATGA[C/T]ACCCACACTTCACTCT | C | 0.6732 | stroke |
| rs1653157 | 18 | 115.203760104642 | CAGGCAAATGTCCAAA[C/T]GAGCATTAGGTGTACG | C | 1.757 | stroke |
| rs16941252 | 15 | 83.2729009279176 | TAAATCAAGTGTTTAA[A/G]GAGGGACATTCACGTC | A | 1.579 | stroke |
| rs17266631 | 13 | 47.1574972192513 | GGGCTAGGGTTTCCTG[C/T]AGTATGCAATAACATT | C | 1.465 | stroke |
| rs1726673 | 1 | 255.995352561274 | CCTGAAAGCCATAAAA[C/T]AACTGCTAAGTTTTTC | C | 0.6956 | stroke |
| rs17347351 | 3 | 152.840605483329 | TTAAATGGCCCTCAAA[A/G]GATACTCCTCTTACAA | G | 1.508 | stroke |
| rs17653276 | 13 | 47.2085614705882 | CTTTAGAAAACAGAGA[C/T]GGTGTGCTTCCAGCAC | T | 1.465 | stroke |
| rs1790866 | 18 | 110.712755334313 | AGGGCCGAAAGAGTAG[A/C]CGAGGGCTGGTGGTCT | A | 0.579 | stroke |
| rs2031817 | 13 | 47.1390694385027 | TGAAATTTATTCTGCA[C/G]GAAATGGGCAAACCAA | C | 1.46 | stroke |
| rs2252884 | 21 | 35.9816334194145 | AGAAATGAGTTGCCCA[C/T]CTAACTAGGGTGATGT | T | 1.437 | stroke |
| rs2282775 | 4 | 64.0030341002257 | ATAGGTTTGTGTTTTA[C/T]CCTTGATCAATTTTGA | T | 0.7577 | stroke |
| rs2363683 | 3 | 80.54272809913 | TTGCCGTCCACAGCAG[G/T]CTACTGACGAAAAACG | T | 1.715 | stroke |
| rs2590577 | 1 | 256.006215812909 | GAAACATTACCATATT[C/G]AAGACAGTGAACAAGT | C | 0.7026 | stroke |
| rs29336 | 4 | 116.896624163051 | AAGGAAGAGTTTGACA[C/T]GAAGTAAAAGTCTCTG | C | 0.7319 | stroke |
| rs3025657 | 6 | 44.3129869718972 | ATATGTTATTCTCTCC[A/C]GGAATTGAATTTACTC | C | 3.691 | stroke |
| rs3824613 | 10 | 67.4903689039669 | CTTTGTGCGAGCGCAG[A/G]CATCCCCATTCCATGC | A | 1.572 | stroke |
| rs4608810 | 4 | 25.6940492819587 | TACAGATCAAGATGTC[A/G]ACAAGGTTGGTTTTCT | G | 1.449 | stroke |
| rs4641492 | 11 | 142.776650083098 | AAGAGATGTATTTCAA[A/G]ATGCGAAATTAACAGT | G | 0.6863 | stroke |
| rs4734805 | 8 | 117.549345185631 | CAGAGCAGACAGCTCC[A/G]GAGAAGAGGAACTCAA | G | 1.519 | stroke |
| rs4766526 | 12 | 119.037288699202 | TGCCAGAGTCATGTGA[A/C]CTTGTGGAGTAGATGC | C | 1.436 | stroke |
| rs5026429 | 8 | 3.48320661566512 | CTGTGCTCAGCTTCAG[A/T]ATCAGCCCAGGGAGAT | T | 1.648 | stroke |
| rs540391 | 6 | 119.720161637538 | AATTCATAACTATAAT[A/G]AGCATCAAGTTAACTA | A | 2.043 | stroke |
| rs6093764 | 20 | 58.7005541360327 | CAACTTACAGATATAC[A/G]ATAGACATGAATAACC | G | 0.6634 | stroke |
| rs609856 | 13 | 47.2449347058824 | CTTAGTTTCTGGATAG[C/T]CCATGATATAGCAGTC | T | 1.48 | stroke |
| rs6413416 | 10 | 67.4869800654378 | TTCAAAACTTACTATA[A/G]CACTGCGGTAATCAAG | A | 1.561 | stroke |
| rs6465147 | 7 | 98.3955100896861 | GGCATTGACATCTTTA[C/T]GAGTGACTTTCCATCC | T | 1.402 | stroke |
| rs6491586 | 13 | 82.7874980639262 | CCCGTCCTTGAGATCG[A/G]ACCTGTCTTTGGGGAT | G | 1.547 | stroke |
| rs6593482 | 10 | 67.4871807287061 | TGTAAGTCATGTATCC[G/T]ATTAGAGACTTCTATC | T | 1.558 | stroke |
| rs6761003 | 2 | 193.987990520512 | GACTAAAATATTCCTA[C/T]GTGTATTCAATCAGTT | C | 2.205 | stroke |
| rs6903130 | 6 | 45.7280236579533 | GTTTTGAAGATAGAGA[A/G]CAAAAGCTGTGAAGAA | G | 0.7165 | stroke |
| rs6933331 | 6 | 44.3082956238212 | TCATAAAGTGAGTTTG[A/G]ATCCTGAAGAAGCCAT | A | 3.574 | stroke |
| rs6963627 | 7 | 157.321328699488 | CTGCCATGAGTTATGA[C/T]TAACCGAGAAAAATGT | T | 0.7406 | stroke |
| rs7093687 | 10 | 67.4855589122908 | AATATTGATGAATCCT[C/G]AAAAAATGCTAGCAAA | G | 1.559 | stroke |
| rs7236071 | 18 | 111.003406124292 | TGATTCTGGGGATAAA[A/G]CTGTGAACCAGATAAA | A | 0.573 | stroke |
| rs751082 | 12 | 36.058784321799 | CATCAACTCACCAGGA[G/T]AGTGAGCACTAAGAAC | T | 1.463 | stroke |
| rs7525955 | 1 | 170.757111937448 | GGAAAAGAAGTTACTA[C/T]AGTTTTGCATAGAAAG | C | 1.379 | stroke |
| rs7607204 | 2 | 52.0021339584778 | GACCCCTTTGGTGAGA[C/G]AATGGTCTTGAAAGTA | G | 1.497 | stroke |
| rs7679643 | 4 | 50.9266613947143 | TTTACCTTTTAACATA[C/T]CTGGAATTGGAATGTA | C | 0.5949 | stroke |
| rs7839694 | 8 | 89.5627459558854 | ATACAGTGTATATTTC[A/G]ATTCTATGGCTAAAGT | A | 4.05 | stroke |
| rs7913948 | 10 | 67.4873669984067 | CCGCCCAGGAGCGCGC[A/G]AAACCTTCTCCACACC | A | 1.556 | stroke |
| rs7919239 | 10 | 67.4998415649195 | CTTCTGATTTCCCCGA[G/T]TCCTAATGAAGTTGAA | T | 1.473 | stroke |
| rs7979656 | 12 | 119.030676687307 | CACATCATTACTGAAA[C/T]GGTTGTAGAATGTTCC | C | 1.434 | stroke |
| rs8047671 | 16 | 97.979302357567 | TTGGGAGGGTAAGTAA[C/T]TGGTTGTGATTGAAAG | T | 0.7398 | stroke |
| rs8081285 | 17 | 78.4530089637003 | GATTCACAATCTACAA[C/G]ACCAGCCCAGCCTATC | G | 1.537 | stroke |
| rs8088357 | 18 | 107.880308119985 | AAACTACACATAGCAA[A/T]CCTCAAATTTGAACAG | T | 1.46 | stroke |
| rs886750 | 7 | 45.9263600028343 | AAGTGCCTCGGATCTC[C/T]ACTTTATCTGTTAATA | T | 1.331 | stroke |
| rs912369 | 13 | 82.6716966848818 | TATCACGTGAATAGAC[A/G]GTAACCTTTAATAACC | A | 1.534 | stroke |
| rs9320994 | 6 | 123.962465883347 | GGTGTATTGCCCATGG[C/T]CAGAGAGGAAATTAAG | T | 1.37 | stroke |
| rs9557510 | 13 | 82.7890106274123 | GCCCTTTGGTATCACA[A/G]AAAACCTCTAAGCCCT | G | 1.559 | stroke |
| rs9900205 | 17 | 85.6137455290831 | TTAATCTCACCATGAA[A/T]AGTCAAAGAGAAGAGG | A | 1.271 | stroke |

TABLE B

| | chromo- | | | Minor | | |
|---|---|---|---|---|---|---|
| rs_ID | some | marshfield | Flanking Sequence | Allele | OR | phenotype |
| rs12443955 | 16 | 73.9930260572249 | GTCTCAGTCTCTAGAC[C/G]AGGTATTCTCAGATTC | G | 0.7835 | myocardial |
| | | | | | | infarction |
| rs331742 | 5 | 132.018816320348 | TAAAACATTTTAACCC[C/G]ATCTATGTTTTAAATT | G | 0.6202 | myocardial |
| | | | | | | infarction |
| rs331783 | 5 | 132.016000716777 | AAAGGGATATAATAGA[C/G]AGAAATGGTTGAAAAA | G | 0.6191 | myocardial |
| | | | | | | infarction |
| rs12657171 | 5 | 68.690560237742 | TGCTGAGTGAACACAA[A/G]CAGTAACCTATGTTCT | A | 0.5208 | myocardial |
| | | | | | | infarction |
| rs12814669 | 12 | 93.670394547187 | TATCATCAGCTACACC[A/G]AACACTCAAGTGTAGC | G | 0.7185 | myocardial |
| | | | | | | infarction |
| rs1389798 | 12 | 93.6878459801516 | CAAATCAGGGCAAAAA[G/T]TCAACAGCTTCTGCCC | G | 0.7154 | myocardial |
| | | | | | | infarction |
| rs1404868 | 12 | 93.6517296770377 | CAGTTCTGGAATAGAG[A/C]ATATTGATTTATGACA | C | 0.7185 | myocardial |
| | | | | | | infarction |
| rs1507214 | 12 | 93.7054893230049 | CTTTTCACTATCATCA[C/T]GAAATTCCTTTGCTTC | C | 0.7269 | myocardial |
| | | | | | | infarction |
| rs1507216 | 12 | 93.6978229336937 | GGGTAAAGATAGGTCA[C/G]ATGAAGTAATCTCCGC | G | 0.7212 | myocardial |
| | | | | | | infarction |
| rs2389316 | 13 | 78.2611130244903 | GGCTCCTCGTTGAAGT[C/T]ACCTCAAGCAGCCTGA | C | 1.32 | myocardial |
| | | | | | | infarction |
| rs2683690 | 2 | 85.9475610179294 | CAAAATCATGGCTCCA[C/T]CTCTGTGATATTTTGA | C | 1.353 | myocardial |
| | | | | | | infarction |
| rs7302315 | 12 | 93.6650009427214 | AGCTCTGCAGACCTGA[C/T]AGATGTGGCAAATACA | T | 0.7185 | myocardial |
| | | | | | | infarction |
| rs7304649 | 12 | 38.3452833944951 | TACAGTTAGGTAAAGA[C/T]AGAGTGGTGAGGGTAA | C | 0.5745 | myocardial |
| | | | | | | infarction |
| rs7421353 | 2 | 48.0629134730808 | TCTGTGCTCTCAATAA[C/T]CCTATATTCTTGTGCG | T | 0.7513 | myocardial |
| | | | | | | infarction |
| rs7843358 | 8 | 66.4240333521283 | TTATTAAACATGATAC[A/C]CAAAGTACAGTGAAAA | C | 0.6178 | myocardial |
| | | | | | | infarction |
| rs7966105 | 12 | 93.7074981832055 | TACAAATAGGACATCA[C/T]GAAAACTGCATCAAAT | T | 0.7269 | myocardial |
| | | | | | | infarction |
| rs16865535 | 2 | 182.78 | GGATGCCCAGTGCATT[G/T]AAAATCGAAACTCTTA | G | 1.534 | myocardial |
| | | | | | | infarction |
| rs10121700 | 9 | 26.33 | AGTTTGATAATGTAAA[C/T]TGGGGAAAGTGAGTAT | T | 1.344 | myocardial |
| | | | | | | infarction |
| rs10827750 | 10 | 64.15 | GAAATAGCCTTACAAC[A/G]CTGTGAATTCAATCCT | G | 1.369 | myocardial |
| | | | | | | infarction |
| rs10880791 | 12 | 59.06 | AAGATCACATGTGTTA[C/T]ACCTTAACGAAAAACT | T | 1.386 | myocardial |
| | | | | | | infarction |
| rs10968433 | 9 | 50.97 | TAGCAGCTCATACAAG[C/G]ATAGGTGATATCACAA | C | 1.385 | myocardial |
| | | | | | | infarction |
| rs10968434 | 9 | 50.97 | ATCAATAGCAGATTCA[A/G]CTTGCCTTTACCATGT | A | 1.376 | myocardial |
| | | | | | | infarction |
| rs11258652 | 10 | 35.24 | TTTTACATCCTTGCTC[G/T]CCTGACTTTCATGAAA | T | 1.287 | myocardial |
| | | | | | | infarction |
| rs12413650 | 10 | 160.05 | GGGCTCTTTGCTTGCA[A/G]ACCAGTTCATCATACC | A | 0.6769 | myocardial |
| | | | | | | infarction |
| rs12695902 | 3 | 164.90 | GGATTATCAGCATTTA[A/G]GCCAGAGTTGCAAATT | G | 0.725 | myocardial |
| | | | | | | infarction |
| rs1280680 | 4 | 130.99 | ACCCAATTTGTTTTTA[C/T]CAGTTGGAACACTTTT | T | 1.394 | myocardial |
| | | | | | | infarction |
| rs1320865 | 2 | 130.51 | CAGATTGCTGAACTTC[A/G]CAAAGGCTAGAGAGCT | A | 1.489 | myocardial |
| | | | | | | infarction |
| rs13316352 | 3 | 172.25 | TAAGCACAATATTGAA[C/G]TATAAAAGACATCTTG | G | 1.476 | myocardial |
| | | | | | | infarction |
| rs1338195 | 1 | 99.28 | ATGGTTCCAAGTTGCC[A/C]ACAACAAATGTCGCAC | C | 0.7372 | myocardial |
| | | | | | | infarction |
| rs1540369 | 2 | 201.17 | CACTCTAATGCACATG[C/G]ACAATAGGCAAGGCAG | G | 1.065 | myocardial |
| | | | | | | infarction |
| rs1862230 | 5 | 95.07 | CAACTATTTAACTGTA[C/T]CTTTGTCGTACAAAAA | T | 0.7278 | myocardial |
| | | | | | | infarction |
| rs1875205 | 16 | 17.57 | CTATTAGTTACTTTCC[A/C]GACTCTCTTCCTCCTC | C | 1.376 | myocardial |
| | | | | | | infarction |
| rs1876681 | 5 | 62.43 | TAAACACTCTCATGGA[A/G]CATCAGTGTCAGACCA | G | 1.507 | myocardial |
| | | | | | | infarction |
| rs2292343 | 17 | 63.91 | CCTGGCTCTAAATTCC[C/G]CCGTATTTTGTAATCT | G | 1.866 | myocardial |
| | | | | | | infarction |
| rs2312150 | 1 | 99.28 | GGTACTTGACTCCAGA[A/T]GACTACAGTGATAATT | A | 0.7372 | myocardial |
| | | | | | | infarction |
| rs2370413 | 12 | 3.30 | CCCTTTGACAGCAAAA[C/T]CTGGCCTTTCCTGAAT | C | 1.105 | myocardial |
| | | | | | | infarction |
| rs2667406 | 4 | 130.99 | ATCATCTGAGAAAAAA[A/G]TATAGACCCAGAACAA | A | 1.397 | myocardial |
| | | | | | | infarction |
| rs4531484 | 11 | 124.82 | CAAATGAGTGTTGTGC[A/C]CTTAAGGAAATTGGCT | C | 1.606 | myocardial |
| | | | | | | infarction |
| rs4693571 | 4 | 91.98 | GAAGAGATGTGGGGGC[A/G]GAAAGACAGGAAATAG | G | 1.762 | myocardial |
| | | | | | | infarction |
| rs4702266 | 5 | 12.59 | TAGTGAGGCTCACAGC[A/G]AGTTAGGTAGATGGGA | G | 0.6995 | myocardial |
| | | | | | | infarction |
| rs4775276 | 15 | 53.26 | ACTATCTTAAGTGCAC[C/T]GAATAATCAACACCAG | T | 1.182 | myocardial |
| | | | | | | infarction |
| rs4937159 | 11 | 127.81 | AAAAATAATAAGCCTA[C/T]CTTATCTCTCTACATG | T | 0.6513 | myocardial |
| | | | | | | infarction |
| rs6452689 | 5 | 95.07 | AATTGTAGCTCTCACA[A/T]TAAAGCTGGGTGAAAA | A | 0.7213 | myocardial |
| | | | | | | infarction |
| rs6478237 | 9 | 124.86 | ACAAAGCCTCCTTTTG[A/C]ATGGATTAGGAAAACA | A | 1.313 | myocardial |
| | | | | | | infarction |
| rs6582553 | 12 | 59.05 | GCTCTAAAGAAACAGC[A/G]CTTGCCTGGGCTTGGC | G | 1.348 | myocardial |
| | | | | | | infarction |
| rs7240443 | 18 | 72.72 | CCTCAGTGACGCTCTA[A/C]CATAGCTTGTTATAGT | A | 0.7775 | myocardial |
| | | | | | | infarction |
| rs8094588 | 18 | 72.79 | AGCACTGACTTCAATA[C/T]GTCCATCAATTTTCCA | C | 0.7774 | myocardial |
| | | | | | | infarction |
| rs9851591 | 3 | 64.16 | CCAGGGCTAGCTCTGG[A/G]CATTTCAAGAGATGTA | A | 0.464 | myocardial |
| | | | | | | infarction |
| rs1413896 | 6 | 48.1033457516153 | AAGGTCACCAAGTTGA[A/G]CATGTATATGGCACAG | A | 1.545 | myocardial |
| | | | | | | infarction |
| rs2960744 | 7 | 151.077317073538 | TGTTTTCATTATGCTA[A/T]GTACAAGATTGTGCTA | T | 0.7146 | myocardial |
| | | | | | | infarction |
| rs3019442 | 6 | 166.369310287848 | TGCATTAAAATCTGTA[C/G]CAGGAATCACTTCTGC | G | 1.284 | myocardial |
| | | | | | | infarction |
| rs6955265 | 7 | 151.078181412071 | ACAGGATCTAGTGCTC[A/G]ATTGCAAGCAGGTGAC | G | 0.7219 | myocardial |
| | | | | | | infarction |
| rs7317235 | 13 | 43.1013612089089 | CTGAAGTCCTTGCTGC[C/T]AGTTCAAACTTGTTCA | C | 0.7376 | myocardial |
| | | | | | | infarction |
| rs10741552 | 11 | 18.01 | AGAGGAAATGATGTGC[A/G]AATGAGAAAATTTTAA | A | 0.5895 | myocardial |
| | | | | | | infarction |
| rs10743478 | 12 | 43.09 | TTTCAATTATGCTCCA[C/T]TGACATAACAGCTGTT | C | 1.2 | myocardial |
| | | | | | | infarction |
| rs1154436 | 4 | 105.54 | TGTTAAATAAATAAAC[C/T]AGGCAGAGAAAGACAA | T | 0.7295 | myocardial |
| | | | | | | infarction |
| rs11577590 | 1 | 97.68 | TTAAACTGGATACCGA[C/G]ACCCAGATTGAGGTGA | C | 1.523 | myocardial |
| | | | | | | infarction |
| rs1177257 | 14 | 42.45 | AAAATCAAACAGGAGA[A/G]CAACGTTACACAGAAT | A | 0.6791 | myocardial |
| | | | | | | infarction |
| rs12505447 | 4 | 129.54 | AGGGGGTTGTGAACCC[A/G]AGGGAACTGCTCTGCC | A | 0.5999 | myocardial |
| | | | | | | infarction |
| rs12750223 | 1 | 99.30 | AATCTAACACAGCTCC[A/G]AGAATTTTCTTCTGGT | G | 0.5517 | myocardial |
| | | | | | | infarction |
| rs12965571 | 18 | 41.69 | TTCAAAAAAAGTGTAC[C/T]ATTTCCATCTGATCAG | T | 1.383 | myocardial |
| | | | | | | infarction |
| rs1603614 | 4 | 60.12 | GTTATGTATCACTTAA[G/T]GACAGAGACACATTAT | T | 0.8021 | myocardial |
| | | | | | | infarction |
| rs1958234 | 14 | 47.84 | TATAACAACGTAGTGA[A/G]ATAGAAGTTATGTGAA | G | 1.512 | myocardial |
| | | | | | | infarction |
| rs221871 | 18 | 80.70 | TGTTTAAACTTATGCA[G/T]TCTTTGGGCCAGTCTT | G | 0.6297 | myocardial |
| | | | | | | infarction |
| rs221873 | 18 | 80.70 | AGTCACTGCACTAATG[C/T]AAAGGCAAGAAGAACA | T | 0.6324 | myocardial |
| | | | | | | infarction |
| rs221878 | 18 | 80.71 | CCAAATGATCTGCCAA[C/T]CCTCTGAATAACCAGC | C | 0.6291 | myocardial |
| | | | | | | infarction |
| rs2449818 | 8 | 4.29 | CTCTAATGATGTAAAA[C/G]GCCTCAGACATCGTTA | C | 0.7775 | myocardial |
| | | | | | | infarction |
| rs2577592 | 2 | 119.50 | AAGGCTGCTCATTTGA[C/T]GGATGAATTACTCCAT | T | 0.6359 | myocardial |
| | | | | | | infarction |
| rs2652148 | 5 | 4.88 | GGTAGGACATTGCTTA[A/G]CTTCAGTGAATCCACA | A | 0.6913 | myocardial |
| | | | | | | infarction |
| rs28490018 | 14 | 114.69 | TCATCTGTGAATGAAG[A/G]ATGTCAATATTCAGGA | A | 0.6316 | myocardial |
| | | | | | | infarction |
| rs3128625 | 9 | 155.14 | CCCACCACTCCACAAC[A/G]AGCCACTAGGAGTTTC | A | 0.722 | myocardial |
| | | | | | | infarction |
| rs4239307 | 18 | 41.83 | TTACATATCAGTTGCT[A/G]AGAATATCTTCTTCCA | A | 1.375 | myocardial |
| | | | | | | infarction |
| rs424301 | 9 | 21.04 | AGAGTGTGCGCTCAAG[C/T]AGGAAAGAAGTAGCAT | T | 1.389 | myocardial |
| | | | | | | infarction |
| rs4296321 | 18 | 41.77 | CAGTGCCATAGGAAGA[A/T]GTGCTCAAGAAGCGTG | T | 1.375 | myocardial |
| | | | | | | infarction |
| rs430208 | 16 | 43.38 | AAGATTTTTAAGCAGC[A/G]GAGTAACATGGTTCAG | G | 1.486 | myocardial |
| | | | | | | infarction |
| rs4341081 | 7 | 151.08 | AGGCTGGGGACTAGTG[C/G]ATCATCGCTTAGGATT | C | 0.6854 | myocardial |
| | | | | | | infarction |
| rs4968008 | 16 | 43.44 | CAAATAAGCAACTTAC[G/T]GAAGGTCATGTGACTC | T | 1.486 | myocardial |
| | | | | | | infarction |
| rs6089599 | 20 | 101.88 | TCCTGGGAACAGTAAC[A/G]CCCCAGTAACACTGAG | G | 0.7165 | myocardial |
| | | | | | | infarction |
| rs6470173 | 8 | 131.44 | AACTGAGACTGCCAAA[C/G]AGATAATCTAGAATCC | C | 1.711 | myocardial |
| | | | | | | infarction |
| rs6814800 | 4 | 57.19 | TAAGGACACAGATTCA[C/T]GGGTGAGCTTTAGGGG | C | 1.439 | myocardial |
| | | | | | | infarction |
| rs6981660 | 8 | 60.15 | TTTCTAATAAATACAC[A/G]ATTCTATGTCAAATTC | G | 0.881 | myocardial |
| | | | | | | infarction |
| rs7067738 | 10 | 35.46 | ACCTGCCTGGTGTCTA[C/T]GATCTGAGACTCTTCT | T | 0.7619 | myocardial |
| | | | | | | infarction |
| rs7136397 | 12 | 59.40 | TAGGGAAGAAAGCCTA[C/T]GGTGGTTATGGAGGGA | T | 1.447 | myocardial |
| | | | | | | infarction |
| rs714875 | 9 | 33.13 | CAGATCCAAGTGTGTT[G/T]AAAAATAGTGTCCCAG | G | 1.668 | myocardial |
| | | | | | | infarction |
| rs7301998 | 12 | 59.40 | GAGTTCCCAACCATGA[C/T]GGGATGTGGGTAGAAC | C | 1.454 | myocardial |
| | | | | | | infarction |
| rs7516762 | 1 | 215.39 | GGATGGAGTTCCAGGC[A/G]CTCTTCAGGTAGTAGC | A | 0.5914 | myocardial |
| | | | | | | infarction |
| rs826686 | 2 | 119.56 | GTTATTTACATAGTTA[C/T]AATGCTCTTTCAAACA | C | 0.6392 | myocardial |
| | | | | | | infarction |
| rs826692 | 2 | 119.57 | ATTTCAGGAAATTTTG[C/T]AATTGAGGCACAAGGA | C | 0.6358 | myocardial |
| | | | | | | infarction |
| rs980373 | 18 | 62.94 | GTGATGGCCCAGAAAA[G/T]AGTAGTGAAGCCAATT | G | 1.442 | myocardial |
| | | | | | | infarction |
| rs9871763 | 3 | 83.22 | TAATTTGAGAGACAAT[A/C]AAACACACAAAAAATG | A | 0.7903 | myocardial |
| | | | | | | infarction |
| rs9965472 | 18 | 52.75 | TCTACCTCTCGTCTTC[C/T]CTAATCAGCTCCTCCT | C | 0.6934 | myocardial |
| | | | | | | infarction |
| rs17191463 | 15 | 53.30 | AGTCCATTTGTGTACA[A/G]TCAGAACCTTTCAACC | G | 1.259 | myocardial |
| | | | | | | infarction |
| rs230157 | 20 | 57.98 | ATGCTGTGTTTTCCAC[A/G]ATACTGGTACTACCGG | A | 1.253 | myocardial |
| | | | | | | infarction |
| rs4679840 | 3 | 172.27 | AAGGATATTTGGACAC[A/G]CTTAATATCTTTTGGA | G | 1.355 | myocardial |
| | | | | | | infarction |
| rs7616299 | 3 | 172.27 | ATGGCGCATATTGAAA[C/T]GAAAAGGATATAGAAA | T | 1.355 | myocardial |
| | | | | | | infarction |
| rs951574 | 16 | 90.99 | AGCCCTGAAAATCAGC[A/G]CAACCTCGGACATCCA | A | 0.4947 | myocardial |
| | | | | | | infarction |
| rs16948951 | 17 | 69.7004393353469 | GGGCCACATGCTGATA[C/G]GTGAGAGGCGGTGGGG | G | 0.7025 | myocardial |
| | | | | | | infarction |
| rs16948981 | 17 | 69.7226771091988 | CTTGAACTGAGTAGGC[C/T]AAAAATTTGCTTTCCT | T | 0.7041 | myocardial |
| | | | | | | infarction |
| rs13173656 | 5 | 165.73 | GTACTCTTATCAGGCA[A/G]GACATTTCCATGGCCT | A | 0.635 | myocardial |
| | | | | | | infarction |
| rs2017914 | 20 | 58.31 | AAATCATATTCCAAAT[A/G]AGCATCAAAAATAATA | G | 0.8261 | myocardial |
| | | | | | | infarction |
| rs6906788 | 6 | 136.84 | TTTGCAATGGTGTTCT[C/T]AAAAATTAGAATGTTA | T | 0.675 | myocardial |
| | | | | | | infarction |
| rs9331931 | 8 | 54.37 | GCACCCAAAGATAAAT[C/G]AATGATGTTACGAAGC | G | 0.7905 | myocardial |
| | | | | | | infarction |
| rs10071774 | 5 | 68.6778492818504 | AGGGCCTTATGTGTTA[C/T]GTTAAGCACCAGGAAC | T | 0.6642 | myocardial |
| | | | | | | infarction |
| rs10471462 | 5 | 68.6773216394885 | ATCCCTGCAAGACCAT[C/T]AATGTAATGAACAGAA | C | 0.6642 | myocardial |
| | | | | | | infarction |
| rs10514925 | 17 | 63.91 | ACGTAGGACGCCGGCA[C/G]AGAAGGAGCCGGGTGG | C | 2.022 | myocardial |
| | | | | | | infarction |
| rs11183154 | 12 | 59.3291099691675 | CATGTCCAAATGACTA[C/T]CTACAGTTCAAACTGC | T | 0.6524 | myocardial |
| | | | | | | infarction |
| rs11848645 | 14 | 37.866943264409 | AGAGAAAATTAAAACA[G/T]TGTTGGCCAAGAATGC | G | 1.225 | myocardial |
| | | | | | | infarction |
| rs11951359 | 5 | 68.6838756257417 | CTGTAAACTTCTATAC[G/T]ACTTTGTACCACCCAT | G | 0.6642 | myocardial |
| | | | | | | infarction |
| rs12458118 | 18 | 76.88 | AATTTTGAGGTGATAC[A/T]AGCATTTAGTCCAAAG | A | 0.7108 | myocardial |
| | | | | | | infarction |
| rs130250 | 22 | 31.0657277970264 | GTGATGGTTGGAGAAA[C/T]GGGCACCTGGCTTTAA | T | 0.7902 | myocardial |
| | | | | | | infarction |
| rs13276054 | 8 | 46.70 | GAGTTGTCAAAGAGGA[C/G]TCACAGCAGAGGAAAA | C | 0.7247 | myocardial |
| | | | | | | infarction |
| rs16825798 | 3 | 186.76 | TGCTGAACAAAATAAT[A/C]ACCGTCTCAAAAATAA | C | 0.6081 | myocardial |
| | | | | | | infarction |
| rs17763040 | 13 | 25.45 | CATGATGTCCTAACAA[A/G]CAAATTATCACTTTAA | G | 0.6599 | myocardial |
| | | | | | | infarction |
| rs243839 | 16 | 73.5593342524906 | AGTCTGAGTTTAATCA[C/T]CCTGCAATACTGTCCT | C | 1.407 | myocardial |
| | | | | | | infarction |
| rs3883317 | 17 | 63.91 | GGAAGAGTCCAAATGT[C/T]AAATATGGTTCCAGGA | T | 2.092 | myocardial |
| | | | | | | infarction |
| rs3917768 | 1 | 187.490323130792 | GCCAAAATTTGGGCGC[A/C]GATTCCACCACTTGGT | C | 1.297 | myocardial |
| | | | | | | infarction |
| rs6956284 | 7 | 151.062258495857 | ACTCTATTCCATTGGA[A/G]ACAAACCTAGGTTGTA | G | 0.7302 | myocardial |
| | | | | | | infarction |
| rs7501838 | 17 | 63.91 | CTGTCAAATCAACTCA[C/T]CTTCAACAAAGGAGTC | C | 2.048 | myocardial |
| | | | | | | infarction |
| rs7750426 | 6 | 166.65 | GATTTCCCTTGTTGAA[A/C]CTTATACACACTTACC | A | 0.8017 | myocardial |
| | | | | | | infarction |
| rs7765427 | 6 | 91.2001534471384 | CAGGATATTGGACACA[A/T]CTGAAGAGTAAATTCA | T | 1.416 | myocardial |
| | | | | | | infarction |

TABLE C

| | chro- | | | Minor | | |
|---|---|---|---|---|---|---|
| | mo- | | | Al- | | |
| rs_ID | some | marshfield | Flanking Sequence | lele | OR | phenotype |
| rs10248275 | 7 | 5.28431449966023 | TATTTTTCGTTTTCAG[A/G]ACATTCAGTTGGTATG | A | 1.896 | albuminuria |
| rs11688740 | 2 | 80.0259360585699 | CTAGGCTTGAATGAGA[A/G]AATAACGATACCTTTC | A | 1.455 | albuminuria |
| rs11707973 | 3 | 152.644548409536 | GCACCTCCTCTGTCCA[A/G]GATATGATGCCAAGTA | G | 0.7492 | albuminuria |
| rs11848645 | 14 | 37.866943264409 | AGAGAAAATTAAAACA[G/T]TGTTGGCCAAGAATGC | G | 1.475 | albuminuria |
| rs12403846 | 1 | 248.325419402142 | GCATGTTTCTAAAAAC[A/G]CTTCAGGAATCCAGAG | A | 0.5789 | albuminuria |
| rs12973797 | 19 | 58.7747686911762 | CACATGTGGGTCGTCA[A/G]CTGCCATCAGGAGTAG | G | 0.6627 | albuminuria |
| rs130250 | 22 | 31.0657277970264 | GTGATGGTTGGAGAAA[C/T]GGGCACCTGGCTTTAA | T | 0.7424 | albuminuria |
| rs16880395 | 4 | 43.2847830924277 | AACTCTGGTGTTGACC[C/T]AGCGGGTAGCATGAGA | T | 0.7031 | albuminuria |
| rs17600042 | 7 | 108.716888412564 | GTAAATGTTTCGAGTA[C/T]GTTATTGGGCTCCATT | T | 0.5263 | albuminuria |
| rs17680407 | 4 | 43.2767428059186 | GAAGTGTTAGTGATGA[C/T]GGACAGAAATAGACAA | T | 0.6885 | albuminuria |
| rs17680905 | 4 | 43.2825226461342 | AGATACAAACCAGATT[C/G]AAAGGATCTTTAATAT | G | 0.6885 | albuminuria |
| rs2171304 | 10 | 7.69100647300684 | GCGTTAGAATTAGAGA[C/G]GAGCCTGTGACGCTTG | C | 0.7056 | albuminuria |
| rs2324872 | 13 | 50.4119178426627 | ATGCAAATGGATCCAA[C/T]ACTTGGTTAGCCAATA | C | 0.725 | albuminuria |
| rs243839 | 16 | 73.5593342524906 | AGTCTGAGTTTAATCA[C/T]CCTGCAATACTGTCCT | C | 1.422 | albuminuria |
| rs2654981 | 15 | 112.357639941574 | TCACATTTCAATGGTA[C/G]GAAAAGTGGCTTCGTA | G | 0.8185 | albuminuria |
| rs2740485 | 9 | 110.556832561452 | TAAATGGTGCCTCTAA[A/C]ATAAAGGGAAATAAAA | C | 1.474 | albuminuria |
| rs2936535 | 8 | 8.48492744170879 | AGCACTTTGCTCTTCC[G/T]CACCAATGCAGTACAG | T | 0.6997 | albuminuria |
| rs3809485 | 15 | 44.9649154987888 | CTTAGCTCTTTGCTTA[C/T]GGTGTTGCCAAATTCC | T | 0.7231 | albuminuria |
| rs470455 | 18 | 52.4650855766638 | CAGATGGTCACTTCAT[C/T]AGCATAAACACTAAGC | C | 0.6921 | albuminuria |
| rs4940275 | 18 | 68.2077595753684 | TAATGATGGGACTCAG[A/G]CATAAGGAAAAAGCAA | A | 0.6717 | albuminuria |
| rs6585304 | 10 | 134.112811120488 | CTAAGCTAACTGAAAA[A/G]ATTGGCATATCACAGG | A | 0.4007 | albuminuria |
| rs6589065 | 11 | 102.332618193842 | AGGGCTGATTATGTTC[C/T]ACCAAGTCACTATGGA | T | 2.541 | albuminuria |
| rs7203172 | 16 | 48.842530986344 | TGAGAACTACCCAACT[A/G]AGCCAAAGTAACCCAC | A | 0.5695 | albuminuria |
| rs7243021 | 18 | 68.2115216788161 | ATATTTCAGCTCCACA[A/G]TGAAATCATTTTATCC | G | 0.7342 | albuminuria |
| rs765 | 15 | 44.9628865477231 | CAGGAACTGCCTTTTA[C/T]GTCTGCAAATAAACTC | C | 0.7533 | albuminuria |
| rs7748736 | 6 | 11.5081596269164 | TGGTCTGAAATTGAGA[C/T]GCTTATTGAATGCTTA | T | 1.346 | albuminuria |
| rs7967892 | 12 | 58.8316738934898 | TGGAGGAAAACCTCAC[C/T]GACTTGGCAACATTTG | C | 1.397 | albuminuria |
| rs9293123 | 5 | 37.3191727499998 | ACCACAAGCAACGGAC[A/T]AGTAGGGGAAGAGTCG | T | 1.407 | albuminuria |
| rs9502593 | 6 | 11.5117329185042 | ATACTTAATTTTTCCA[A/G]TATGGTCTAGGTTAGA | G | 1.343 | albuminuria |
| rs10514925 | 17 | 63.91 | ACGTAGGACGCCGGCA[C/G]AGAAGGAGCCGGGTGG | C | 2.066 | albuminuria |
| rs16825798 | 3 | 186.76 | TGCTGAACAAAATAAT[A/C]ACCGTCTCAAAAATAA | C | 0.5849 | albuminuria |
| rs3883317 | 17 | 63.91 | GGAAGAGTCCAAATGT[C/T]AAATATGGTTCCAGGA | T | 2.16 | albuminuria |
| rs7501838 | 17 | 63.91 | CTGTCAAATCAACTCA[C/T]CTTCAACAAAGGAGTC | C | 2.153 | albuminuria |
| rs1218661 | 1 | 262.1 | AGACTCAAAGATAGAG[A/C]CCAAAAAAGTGACACT | A | 1.342 | albuminuria |
| rs1327210 | 13 | 50.4 | TTCTAGTGCTTCAGAA[G/T]GAATAAAAGGTCATAG | T | 0.6847 | albuminuria |
| rs13333226 | 16 | 38.9 | GTAGCACAGCTGTAGG[A/G]ATATTGACTCCTCTTC | G | 0.6717 | albuminuria |
| rs1364406 | 7 | 142.5 | CAATGTTAAGCACAGA[C/G]AGTTGAGTAGCTTCTT | C | 0.8007 | albuminuria |
| rs1913695 | 12 | 58.8 | TATTTGTGAATTGCCA[C/T]GCCAAAAGAAAATAAC | T | 1.353 | albuminuria |
| rs2962394 | 5 | 163.1 | TTCCTCCTAGGAGTGA[C/G]AGTGAAATAATCTGAA | G | 0.7377 | albuminuria |
| rs4378283 | 10 | 82.4 | ATTCATTATTGCCAAA[A/G]ATTTCTAGAGGACATG | A | 0.4838 | albuminuria |
| rs4572098 | 11 | 93.3 | CTGTCGTGTTTAGGAA[C/T]GTCCACCTATTACTGT | C | 0.6758 | albuminuria |
| rs4936167 | 11 | 141.0 | TCATTACAGCAGCTAA[C/T]GTAGGCTAAGCCCACA | T | 0.523 | albuminuria |
| rs617595 | 13 | 59.8 | GAAACTGAGGCACAGT[A/G]AACTAATTTTCCCAAG | A | 1.455 | albuminuria |
| rs634585 | 13 | 50.4 | TGAGTGAGTTACCATA[C/G]AACCTGAGCTGCCCAT | G | 0.6847 | albuminuria |
| rs9287229 | 1 | 262.1 | CCCTTATCTTTGCTCC[A/G]GACTAACCTGTGGCTG | G | 1.347 | albuminuria |
| rs9540649 | 13 | 50.4 | TTGCTTACAGGGAAAC[A/G]CTATCTGAGACAAGCT | G | 0.6836 | albuminuria |
| rs980907 | 13 | 50.4 | CTACCAACACTGTGAG[A/G]AAATTTGGATATGGAA | A | 0.6797 | albuminuria |
| rs7947110 | 11 | 12.668172241806 | CCTATAGGAAACACCA[A/G]TAGTGACCTACACAGC | A | 1.3 | albuminuria |
| rs10804520 | 3 | 130.6 | CTCTTCTGTGGGAAGC[A/G]AAAACACTGTGCACCT | A | 1.474 | low |
| | | | | | | creatinine |
| | | | | | | clearance |
| rs10922204 | 1 | 204.5 | GGAGGATATTAAAGAC[G/T]GAACTACCACAATTTC | T | 2.566 | low |
| | | | | | | creatinine |
| | | | | | | clearance |
| rs12127748 | 1 | 204.5 | ACGTGGATGTGAGGTA[C/T]GTTACCAAATAACTTC | T | 2.738 | low |
| | | | | | | creatinine |
| | | | | | | clearance |
| rs1549840 | 2 | 76.6 | AGCACATCCTATTCTC[C/T]AGTCCCGCGGGCGGCT | T | 1.374 | low |
| | | | | | | creatinine |
| | | | | | | clearance |
| rs16830489 | 1 | 204.4 | CACTTATGTCAGAGCT[G/T]AAGTCAGTGAACCAAA | T | 2.796 | low |
| | | | | | | creatinine |
| | | | | | | clearance |
| rs6666581 | 1 | 204.5 | AAGCACCATATTTGTC[C/T]AGAAAAACCACCTCTC | C | 2.806 | low |
| | | | | | | creatinine |
| | | | | | | clearance |
| rs6673397 | 1 | 204.5 | GGCCTGTCAAATGCAA[A/G]TGCGTATGTGAATGTG | A | 2.595 | low |
| | | | | | | creatinine |
| | | | | | | clearance |
| rs902891 | 12 | 159.6 | CAGGATGTGCCTTAGG[C/T]ATAATGTCAAATGGGC | C | 1.393 | low |
| | | | | | | creatinine |
| | | | | | | clearance |
| rs3917768 | 1 | 187.490323130792 | GCCAAAATTTGGGCGC[A/C]GATTCCACCACTTGGT | C | 1.469 | low |
| | | | | | | glomerular |
| | | | | | | filtration |
| rs4235846 | 6 | 98.8841257773463 | ACAAGTGTAACAGAAA[A/T]GGAATGACAGAAAAAA | T | 1.58 | low |
| | | | | | | glomerular |
| | | | | | | filtration |
| rs10122952 | 9 | 112.5 | GATGTGGGAGAAGGAA[A/G]GTTGTCCTGGGAATTA | A | 1.754 | low |
| | | | | | | glomerular |
| | | | | | | filtration |
| rs10503257 | 8 | 5.1 | GTTTTACCAATAATCA[A/C]CATAAACCTAAAACTA | A | 1.344 | low |
| | | | | | | glomerular |
| | | | | | | filtration |
| rs10748721 | 10 | 120.4 | TAAAGAAAAACATGGA[A/G]GACTTAGCATTCAGAG | A | 0.671 | low |
| | | | | | | glomerular |
| | | | | | | filtration |
| rs10786405 | 10 | 120.4 | GATTTAGTGCCCATGA[C/T]GATGATGAATTATTAG | T | 0.671 | low |
| | | | | | | glomerular |
| | | | | | | filtration |
| rs10816423 | 9 | 112.5 | ATCTGGCCTGAAGGAA[A/T]TCACTGTAGTGAGGAA | T | 1.754 | low |
| | | | | | | glomerular |
| | | | | | | filtration |
| rs10816424 | 9 | 112.5 | CCAGAGTCAGCGCCCA[A/T]TATATTAAATAGAGAG | A | 1.774 | low |
| | | | | | | glomerular |
| | | | | | | filtration |
| rs10922207 | 1 | 204.5 | AAGAAAAATTCAGTCA[C/T]ACAAAACAAAGAAAAT | T | 3.067 | low |
| | | | | | | glomerular |
| | | | | | | filtration |
| rs10978601 | 9 | 112.5 | CAGGACTTAGGTTCAG[C/T]ATGAGGAAAATATCTC | T | 1.754 | low |
| | | | | | | glomerular |
| | | | | | | filtration |
| rs10978606 | 9 | 112.5 | TCCCGATAAATCGTGT[A/C]AATTATTTGCTCAGCC | C | 1.774 | low |
| | | | | | | glomerular |
| | | | | | | filtration |
| rs10978612 | 9 | 112.5 | ACACCATAATTAAAAA[A/G]TTGGTCATTTGTTCCA | A | 1.76 | low |
| | | | | | | glomerular |
| | | | | | | filtration |
| rs1292081 | 1 | 56.4 | CTATAAAGTGGCTGCA[C/T]GGATGAGGGACATTAT | C | 0.5353 | low |
| | | | | | | glomerular |
| | | | | | | filtration |
| rs1413618 | 10 | 120.3 | GAAAATGTTATCAGGA[A/G]AGCAGGATTAAATAAG | A | 0.651 | low |
| | | | | | | glomerular |
| | | | | | | filtration |
| rs16901596 | 8 | 138.2 | GTGCTGGAGTAATAGG[C/T]CAAATGTATCATGGCC | T | 3.429 | low |
| | | | | | | glomerular |
| | | | | | | filtration |
| rs16901597 | 8 | 138.2 | AGCTCTAATATTTATA[A/G]TACCCACCACGGTTGT | A | 3.338 | low |
| | | | | | | glomerular |
| | | | | | | filtration |
| rs17154065 | 10 | 35.2 | AGGTCTTCGGGGTTTC[C/T]ATCTGAGGGTGTATGA | T | 1.195 | low |
| | | | | | | glomerular |
| | | | | | | filtration |
| rs17692715 | 6 | 85.8 | GACCTTTTCTGATCAA[A/G]CCCTGTGTATGGCTGA | G | 0.7101 | low |
| | | | | | | glomerular |
| | | | | | | filtration |
| rs17770541 | 18 | 83.9 | TCCTCCTTCTGTTTAA[C/T]ACATTTTTCCATCTCA | T | 0.6534 | low |
| | | | | | | glomerular |
| | | | | | | filtration |
| rs1965193 | 12 | 52.7 | AAAAAGAAAAAGAAAA[C/T]TGAGCTTATAGTACAG | T | 1.535 | low |
| | | | | | | glomerular |
| | | | | | | filtration |
| rs2009111 | 22 | 22.7 | CTTTCACTTCACACTG[A/G]ACCCTACAAATGACGT | G | 0.6804 | low |
| | | | | | | glomerular |
| | | | | | | filtration |
| rs2057028 | 20 | 60.4 | AAAAAGAATTCACCCA[A/C]GCTTACATAGCTAGTA | C | 1.845 | low |
| | | | | | | glomerular |
| | | | | | | filtration |
| rs2066926 | 22 | 28.6 | ATTGCAGATTACATAA[C/T]TGGGATTAAGTGAGGT | C | 1.646 | low |
| | | | | | | glomerular |
| | | | | | | filtration |
| rs2444242 | 11 | 116.2 | GGAGTCCAGTCGTTAG[C/T]AGGAGTTCTGGAGGAG | T | 1.998 | low |
| | | | | | | glomerular |
| | | | | | | filtration |
| rs2621459 | 4 | 101.9 | AACCACCATCATCTCA[C/G]TATCTCTTGTCTTGTA | G | 1.421 | low |
| | | | | | | glomerular |
| | | | | | | filtration |
| rs2743979 | 1 | 9.9 | ATTAGTATACAGTGGC[A/G]ATTAACAGAAGCAGAA | A | 0.6528 | low |
| | | | | | | glomerular |
| | | | | | | filtration |
| rs2851870 | 18 | 83.9 | GAAAGTGCCAGGGGAA[C/G]TGCAACTTGGCAATGG | G | 0.6622 | low |
| | | | | | | glomerular |
| | | | | | | filtration |
| rs3750595 | 10 | 120.4 | GCAGAGATAGCAGAAA[G/T]CAAGAAAAAGGTTGGA | T | 0.671 | low |
| | | | | | | glomerular |
| | | | | | | filtration |
| rs41530345 | 9 | 66.0 | GAAGCTATTAGTTTTA[C/T]GCCACTTCATAGAGAG | T | 1.881 | low |
| | | | | | | glomerular |
| | | | | | | filtration |
| rs4285402 | 7 | 151.2 | TTGTAGCTAGGATCCC[A/G]GATGTCTGCAGTGCAA | G | 0.5825 | low |
| | | | | | | glomerular |
| | | | | | | filtration |
| rs4923872 | 15 | 38.3 | GAAACATAAGCTCTTA[C/T]AGGTCTAAAGCCTGCC | T | 1.487 | low |
| | | | | | | glomerular |
| | | | | | | filtration |
| rs6429453 | 1 | 275.7 | CAACTGTAGTGGATAA[C/T]GTCAAGTTGTTTTTCT | T | 1.792 | low |
| | | | | | | glomerular |
| | | | | | | filtration |
| rs645170 | 19 | 34.6 | CACTGGCCTGGGGTAG[G/T]AGCTAAAGCCATATGC | G | 1.783 | low |
| | | | | | | glomerular |
| | | | | | | filtration |
| rs6715897 | 2 | 232.3 | ATTCTGTTTTTAGTTC[A/G]AGGGCCATTGTTTATT | A | 0.5592 | low |
| | | | | | | glomerular |
| | | | | | | filtration |
| rs6750269 | 2 | 232.3 | AATGTAGAGCCTGAGA[A/G]CCTAAGGAAGTATCTG | G | 0.5555 | low |
| | | | | | | glomerular |
| | | | | | | filtration |
| rs745165 | 20 | 107.0 | TGCTGCAGTTATGTAG[A/C]AAGCAGGTTGTCATGG | C | 1.89 | low |
| | | | | | | glomerular |
| | | | | | | filtration |
| rs7556846 | 2 | 232.3 | TGAGAGAACTCCAAGA[A/G]GGATGTCGAAACTGGG | A | 0.5555 | low |
| | | | | | | glomerular |
| | | | | | | filtration |
| rs7570424 | 2 | 232.3 | GCACAGAGTAGAGGCA[C/T]GGTAACGATGTGTTGT | C | 0.5555 | low |
| | | | | | | glomerular |
| | | | | | | filtration |
| rs7609541 | 2 | 232.3 | AGTGTTCTGGTCTGGA[C/T]GAAATGAGAGAACTCC | T | 0.5555 | low |
| | | | | | | glomerular |
| | | | | | | filtration |
| rs7972260 | 12 | 143.9 | TTGTCATAATAACTAC[A/C]ATTTATAGAGTGCCTA | C | 1.5 | low |
| | | | | | | glomerular |
| | | | | | | filtration |
| rs928579 | 10 | 120.3 | CCCTGCCCTAATCACC[A/G]AGCAGACAGGCAACAT | A | 0.6477 | low |
| | | | | | | glomerular |
| | | | | | | filtration |
| rs9426484 | 1 | 10.0 | GGGGCCCTGTTGGTTC[A/G]CTGATCATGAGAACCT | A | 0.6646 | low |
| | | | | | | glomerular |
| | | | | | | filtration |
| rs603151 | 1 | 26.3 | CCCACAGGGGGTGGAA[C/G]GACAAGCAAGTAACCA | C | 1.306 | low |
| | | | | | | glomerular |
| | | | | | | filtration |
| rs7765427 | 6 | 91.2001534471384 | CAGGATATTGGACACA[A/T]CTGAAGAGTAAATTCA | T | 1.618 | low |
| | | | | | | glomerular |
| | | | | | | filtration |
| rs1379666 | 6 | 88.63 | CAGAGGGGAAAGCACA[A/G]TAGACAAGGTCAGCCT | A | 1.535 | low |
| | | | | | | glomerular |
| | | | | | | filtration |
| rs10155009 | 3 | 96.8 | AATCCTCATCCCCAGC[A/G]AATAATTATTGAGTCC | G | 0.6604 | low |
| | | | | | | glomerular |
| | | | | | | filtration |
| rs1161262 | 4 | 95.2 | TCATTTATTAAAACAC[A/G]GCATGATTAGACCACT | G | 2.032 | low |
| | | | | | | glomerular |
| | | | | | | filtration |
| rs1161263 | 4 | 95.2 | CAGTAGTTAGAATAGA[A/G]ATAAGTTGTGGCTTCA | G | 2.078 | low |
| | | | | | | glomerular |
| | | | | | | filtration |
| rs11927384 | 3 | 152.6 | ATCAGATGGTATTTGT[C/T]AACTTACTGCTCTGAA | T | 0.6215 | low |
| | | | | | | glomerular |
| | | | | | | filtration |
| rs12446492 | 16 | 39.0 | AAGTTCTTATTACGCA[A/T]CAGTCACTGTGCTAAG | A | 0.6328 | low |
| | | | | | | glomerular |
| | | | | | | filtration |
| rs1258241 | 10 | 69.7 | AACTACCTCTACTAAC[C/T]ACCATCTAAGCATGGA | T | 0.6556 | low |
| | | | | | | glomerular |
| | | | | | | filtration |
| rs1258246 | 10 | 69.8 | AGCATGGGCCTGAATC[A/C]AAAATGACTGGCATTC | A | 0.6565 | low |
| | | | | | | glomerular |
| | | | | | | filtration |
| rs1258323 | 10 | 69.7 | GGATGTTCTGTTTAGA[A/G]GAAGTTAAGAGCTGAG | A | 0.6689 | low |
| | | | | | | glomerular |
| | | | | | | filtration |
| rs12895324 | 14 | 55.4 | ATGCACAGCACGGTTG[C/T]AGATTTTTAACATTAG | T | 0.6806 | low |
| | | | | | | glomerular |
| | | | | | | filtration |
| rs1332457 | 9 | 129.2 | TGATTAGGAAATGTCA[A/T]GGATACGTCTGTAAAA | T | 0.6871 | low |
| | | | | | | glomerular |
| | | | | | | filtration |
| rs1334237 | 13 | 41.4 | TTGTGAAATAACTGTC[C/T]CAGACTCTTCAAAAAT | C | 1.571 | low |
| | | | | | | glomerular |
| | | | | | | filtration |
| rs17272796 | 3 | 41.8 | CTTACATTCTCTAATA[C/T]GAGTTGAGGTGCCTAG | C | 1.458 | low |
| | | | | | | glomerular |
| | | | | | | filtration |
| rs2088707 | 12 | 144.0 | CTGTTTACTTGCTCCA[A/G]TCTGGCCACACTGGCC | A | 1.835 | low |
| | | | | | | glomerular |
| | | | | | | filtration |
| rs2147102 | 14 | 55.4 | TAAGCAAAGCCAGGAT[A/C]ATCATTCTTATTCCAT | A | 0.677 | low |
| | | | | | | glomerular |
| | | | | | | filtration |
| rs2292927 | 8 | 159.2 | GGTCATCGACCAGTAC[A/G]GCATCCCACAGGTAAG | A | 1.625 | low |
| | | | | | | glomerular |
| | | | | | | filtration |
| rs2413429 | 22 | 42.0 | GGGATGAATGAGTTCA[A/G]AGTCTTGCAAAAGGGA | A | 0.6691 | low |
| | | | | | | glomerular |
| | | | | | | filtration |
| rs2873929 | 4 | 77.5 | ACAAAAAACTAAAATA[C/G]AGTCCATTTGGTCCAG | C | 1.458 | low |
| | | | | | | glomerular |
| | | | | | | filtration |
| rs304215 | 3 | 84.4 | CCCATCCTTTAAACAG[A/G]AACAGTTTGTGAAGAT | A | 0.8263 | low |
| | | | | | | glomerular |
| | | | | | | filtration |
| rs3181 | 11 | 111.5 | AACCTTTGTATGAGCC[C/T]ATTAGCTGCCTCAGCT | T | 1.534 | low |
| | | | | | | glomerular |
| | | | | | | filtration |
| rs355460 | 4 | 77.5 | TATATGTGAAATACCC[A/G]AACTTGGAATGCATTT | A | 1.464 | low |
| | | | | | | glomerular |
| | | | | | | filtration |
| rs355483 | 4 | 77.5 | TATTTCTCTACTGTGC[C/T]GATACTGTGTCCACAC | T | 1.458 | low |
| | | | | | | glomerular |
| | | | | | | filtration |
| rs3909788 | 12 | 125.5 | AACATCCAACAACAAA[G/T]ATCCAATTAAATAAAG | T | 0.6411 | low |
| | | | | | | glomerular |
| | | | | | | filtration |
| rs4258086 | 9 | 49.5 | ACCTCTCACCTCAGCA[A/G]ATTCTTCAGTAATAAT | A | 0.6953 | low |
| | | | | | | glomerular |
| | | | | | | filtration |
| rs4269885 | 11 | 97.8 | AAACTTCACATCATTA[A/G]AAACATTCGGCTGAGT | A | 1.384 | low |
| | | | | | | glomerular |
| | | | | | | filtration |
| rs4280249 | 16 | 38.9 | AGCTGTTCTGTACTTA[A/C]GTCACACCCAGAATTG | A | 0.6218 | low |
| | | | | | | glomerular |
| | | | | | | filtration |
| rs4321205 | 16 | 38.9 | ATGGACAGTCTTACAA[C/T]TCTGGGTGTGACTTAA | T | 0.6198 | low |
| | | | | | | glomerular |
| | | | | | | filtration |
| rs4334629 | 3 | 41.8 | CAAGGACGAAAGATAC[A/G]CAACCAAGGGAATCAC | A | 0.6943 | low |
| | | | | | | glomerular |
| | | | | | | filtration |
| rs4369676 | 16 | 38.9 | TTCAGAGTGATTGTAA[A/G]AGGCCTGCATGATGCA | A | 0.6218 | low |
| | | | | | | glomerular |
| | | | | | | filtration |
| rs4497678 | 16 | 110.5 | AAACAAAAATAAGCGG[C/G]CCCCATCTTAAATCTT | C | 1.462 | low |
| | | | | | | glomerular |
| | | | | | | filtration |
| rs4535211 | 3 | 41.8 | TAAGGATGTCGTGTAA[A/G]CATATCATTAATGTAG | A | 1.467 | low |
| | | | | | | glomerular |
| | | | | | | filtration |
| rs4597881 | 4 | 77.5 | GGAATGAAATAATGGA[C/T]GGTATTTGCAGCAACC | T | 1.455 | low |
| | | | | | | glomerular |
| | | | | | | filtration |
| rs6446391 | 4 | 11.0 | GTGACTTAACGTAGTA[C/T]GAGAACTGTTGAGCTT | C | 1.753 | low |
| | | | | | | glomerular |
| | | | | | | filtration |
| rs6701181 | 1 | 262.8 | TCCTGTCTCAAAGAGG[C/T]AATATAGTGTGAATTG | T | 0.6635 | low |
| | | | | | | glomerular |
| | | | | | | filtration |
| rs6809087 | 3 | 41.8 | CACTGCCTTAGAACAT[A/G]AGTGCTTCCATACAGA | A | 1.459 | low |
| | | | | | | glomerular |
| | | | | | | filtration |
| rs6810410 | 4 | 77.5 | GCCTAACAACAGAGTA[C/T]ACATGAGCCAAAATCT | T | 1.453 | low |
| | | | | | | glomerular |
| | | | | | | filtration |
| rs7194394 | 16 | 107.5 | TGTGCGCAGAAGCAGC[A/C]GATTAACGGAAGAACA | C | 1.712 | low |
| | | | | | | glomerular |
| | | | | | | filtration |
| rs7217945 | 17 | 9.2 | GCAGTGGCTACATTCC[A/G]GAAAACAGCCGCAGAA | A | 0.784 | low |
| | | | | | | glomerular |
| | | | | | | filtration |
| rs731017 | 1 | 151.1 | AAATAAATTGAATTGG[C/T]CAGTCTAGCTTGCAAA | T | 1.489 | low |
| | | | | | | glomerular |
| | | | | | | filtration |
| rs7549770 | 1 | 151.1 | AAAAAGTGAGAGTGGC[A/G]AAAAGTATACTTGATA | G | 1.453 | low |
| | | | | | | glomerular |
| | | | | | | filtration |
| rs7655850 | 4 | 77.5 | AAATACTCAACTCCGA[A/C]TAGCTCTAGTGTTTCA | A | 1.458 | low |
| | | | | | | glomerular |
| | | | | | | filtration |
| rs7690478 | 4 | 57.3 | AGTCTACAAACAACAC[A/G]GAGTAATAACATTATA | A | 1.473 | low |
| | | | | | | glomerular |
| | | | | | | filtration |
| rs7695033 | 4 | 95.2 | ATGCAAAATGCACATA[G/T]TAGTAATTAGTCAAAC | T | 2.126 | low |
| | | | | | | glomerular |
| | | | | | | filtration |
| rs7935913 | 11 | 111.5 | AGGCACTTAGCATACA[A/G]CAATTAAGATGGAAGT | A | 1.513 | low |
| | | | | | | glomerular |
| | | | | | | filtration |
| rs7973993 | 12 | 112.5 | AGATAACCGAAGCTAA[C/T]TGACCACATCCTACTT | T | 2.115 | low |
| | | | | | | glomerular |
| | | | | | | filtration |
| rs9343494 | 6 | 88.6 | ATATGGTAGGACCTTA[C/T]GTGACTGAAGCTATCT | C | 1.536 | low |
| | | | | | | glomerular |
| | | | | | | filtration |
| rs963264 | 1 | 151.1 | ATTCTGAGATCTTTCC[A/G]CTATTTCAGGCTCTAG | A | 1.496 | low |
| | | | | | | glomerular |
| | | | | | | filtration |
| rs965574 | 1 | 151.1 | GCCTGCAGCTAGGCTA[C/G]AGAGAGTTCTGGAAGA | C | 1.479 | low |
| | | | | | | glomerular |
| | | | | | | filtration |
| rs16910240 | 9 | 102.499333039659 | GTCACTCCCTGTCCAA[A/G]TCAAACCTGGATTCTG | A | 2.17 | macro- |
| | | | | | | albuminuria |
| rs2350786 | 7 | 142.552293430087 | CATGGAGGCAAACTAC[A/G]GTAGCTGATTACCATT | A | 1.675 | macro- |
| | | | | | | albuminuria |
| rs7029315 | 9 | 102.499292509778 | GAAAATATTTGAGGAT[A/G]AATGTCTAAATGCAAG | A | 2.175 | macro- |
| | | | | | | albuminuria |
| rs7047415 | 9 | 102.496925050084 | GAAGCAACTTAGTTTC[C/T]CAATGAAAGCTGTAAC | T | 2.179 | macro- |
| | | | | | | albuminuria |
| rs7807871 | 7 | 142.543957387292 | TTCCACAAATTGTTAA[A/T]CAAACTTTCTGTAGAA | A | 1.659 | macro- |
| | | | | | | albuminuria |
| rs1002169 | 7 | 3.0 | TCCCTCCACAGCCACA[C/T]GTTGCTTTTAGCTTGC | T | 0.5485 | macro- |
| | | | | | | albuminuria |
| rs10492295 | 12 | 94.1 | TCAAAGGAATTTTGCA[C/G]GTATTTACATCTCTCT | C | 3.406 | macro- |
| | | | | | | albuminuria |
| rs10780235 | 9 | 78.3 | CCTGATCCTCTGTAAA[C/G]CCTTAATGAATGTAAT | C | 1.973 | macro- |
| | | | | | | albuminuria |
| rs10858809 | 12 | 94.7 | TGCATTTGTTAAAGGC[C/T]GATGTGCTTCTTAACA | C | 1.661 | macro- |
| | | | | | | albuminuria |
| rs10863962 | 1 | 221.9 | TCTCCTTCCTCCAAAC[A/G]GCTCTAATACTTTCAT | A | 1.578 | macro- |
| | | | | | | albuminuria |
| rs10867190 | 9 | 78.3 | ACTGTCTTTTCAAGCA[A/G]GTTCAAACAAGCCTGA | A | 2.113 | macro- |
| | | | | | | albuminuria |
| rs10867195 | 9 | 78.3 | ATCATCCACCAAGCAG[A/G]CATCTTTCCTGAGAAG | A | 2.153 | macro- |
| | | | | | | albuminuria |
| rs10935316 | 3 | 152.3 | ACACACCAAGCTCATC[C/G]ACCCCAGTATCCCACT | C | 1.66 | macro- |
| | | | | | | albuminuria |
| rs12000827 | 9 | 65.5 | TTTCATTCTGAGATGC[A/G]AAATCATTGAGCAGAA | A | 2.314 | macro- |
| | | | | | | albuminuria |
| rs12119983 | 1 | 221.9 | CATTCCTGAATGTTTG[A/C]AAATAGAAGAGGCTTC | C | 1.583 | macro- |
| | | | | | | albuminuria |
| rs1240385 | 10 | 107.4 | AAGGCGCTAGAGAGTA[C/T]GACTTGTGGCTGTTAG | C | 2.834 | macro- |
| | | | | | | albuminuria |
| rs1411441 | 9 | 65.5 | CCACAGTGAGGCTAAA[C/T]ACCCTGGATCTCTTGG | C | 2.439 | macro- |
| | | | | | | albuminuria |
| rs1411442 | 9 | 65.5 | TATTTGTGGGTTCATA[C/G]ATGATTTTTCAGATGT | G | 2.439 | macro- |
| | | | | | | albuminuria |
| rs1455857 | 7 | 142.5 | GTCAGGATGCATTTCA[A/G]AATCATGAGAAAGAAG | A | 1.568 | macro- |
| | | | | | | albuminuria |
| rs1483737 | 4 | 101.9 | AAAGGGATTGTGAAAA[C/T]TGTTAATGAGAAAGCA | C | 1.555 | macro- |
| | | | | | | albuminuria |
| rs1489922 | 3 | 172.8 | TAGAACCAGAGCAGAA[C/T]GTAGCCTCAGGAAGAA | C | 0.6048 | macro- |
| | | | | | | albuminuria |
| rs16931308 | 8 | 81.2 | CTCTCTCATTTTCTCA[A/G]GGAACTCTTCTGCATC | A | 2.078 | macro- |
| | | | | | | albuminuria |
| rs17031374 | 3 | 19.8 | TATGTAAGGTGGGCTA[A/G]AGAAATGTTTAAAAAG | G | 1.771 | macro- |
| | | | | | | albuminuria |
| rs17095305 | 11 | 98.2 | GGTTCTCAGATGAAGA[A/G]GTCATAAGCTACCTGG | A | 3.497 | macro- |
| | | | | | | albuminuria |
| rs17186040 | 13 | 27.7 | TTACTTTAAATGTACA[C/G]AACAATCTGGCTAAGT | G | 0.6951 | macro- |
| | | | | | | albuminuria |
| rs17705657 | 19 | 60.3 | GTTTCTGCTCTTTCTA[A/C]CGCGTGATAGCGGCGC | A | 1.669 | macro- |
| | | | | | | albuminuria |
| rs17721390 | 8 | 87.5 | GGAATACAGTATCATA[C/G]AGACATGGTTTTTTAT | C | 0.2926 | macro- |
| | | | | | | albuminuria |
| rs184255 | 9 | 131.4 | TCTTTGCAATTTTGAA[C/T]CATCTAATTCTACCTA | T | 4.265 | macro- |
| | | | | | | albuminuria |
| rs1966908 | 2 | 103.3 | TACCATTTCAAATTCA[C/G]TATTCAAGCCATATAT | G | 0.5868 | macro- |
| | | | | | | albuminuria |
| rs1966909 | 2 | 103.3 | TATAGCTCTTTGGTAA[C/T]AACAGAAAGGTCATTT | T | 0.5897 | macro- |
| | | | | | | albuminuria |
| rs2026692 | 13 | 44.4 | AAAGGAACTATTCTTC[A/G]CTACACGAGCACTAAA | A | 1.539 | macro- |
| | | | | | | albuminuria |
| rs2114913 | 12 | 108.9 | GGAAAGCAACGAAAGC[A/G]ACGGCAGTTGGACTTC | G | 2.354 | macro- |
| | | | | | | albuminuria |
| rs2220521 | 3 | 172.8 | AATCTCAGATCATTTA[A/C]CTACATCAAACATCTC | A | 0.5941 | macro- |
| | | | | | | albuminuria |
| rs3950018 | 13 | 92.6 | CAATTGATAGAGAAAA[A/T]CACAGAGCTCTCCACA | T | 0.4884 | macro- |
| | | | | | | albuminuria |
| rs4290350 | 13 | 89.7 | ACACTGATTCTGCAGA[G/T]AGTTGGCAATTTCTTC | T | 0.602 | macro- |
| | | | | | | albuminuria |
| rs4331429 | 19 | 82.7 | AGGCAAAAGAGATAGC[A/G]AGAGCGGTGGGGGGTT | G | 1.638 | macro- |
| | | | | | | albuminuria |
| rs4773709 | 13 | 72.3 | CAAAGAACGCTTAATG[G/T]AAGGAAGAAGTATTAG | T | 1.65 | macro- |
| | | | | | | albuminuria |
| rs4790200 | 17 | 10.9 | TTGACATATGCACAGA[G/T]GATCTCTTTTGATTCT | G | 2.124 | macro- |
| | | | | | | albuminuria |
| rs4883520 | 12 | 168.2 | TTGCTGAGTAAGCAAA[C/T]ATAGCATTTTTCTTCA | T | 0.5957 | macro- |
| | | | | | | albuminuria |
| rs6478513 | 9 | 131.5 | CAGAGTGTTAAAGTAA[C/G]TGGCCTAAGGTGTACA | G | 4.265 | macro- |
| | | | | | | albuminuria |
| rs6673199 | 1 | 127.5 | GCCACACCCTTAGCCA[A/G]TCTTGGGCTGGATTTC | G | 2.016 | macro- |
| | | | | | | albuminuria |
| rs7047148 | 9 | 78.3 | ATGAGTCTTTCCCACA[A/G]TGCCTAAAAAGATTCT | G | 2.123 | macro- |
| | | | | | | albuminuria |
| rs7123971 | 11 | 98.2 | AATTCTTTTGGCAATA[C/T]ATCTAGAAATGGAGTT | C | 3.364 | macro- |
| | | | | | | albuminuria |
| rs7464175 | 8 | 77.9 | TGCAGCCTCTGCAACC[G/T]ACTTCCGTGTGCTTTC | G | 0.6016 | macro- |
| | | | | | | albuminuria |
| rs7747972 | 6 | 82.2 | ATCTATATTGAATCCA[A/G]CATCTTAAGTTGCCTG | A | 1.559 | macro- |
| | | | | | | albuminuria |
| rs7761326 | 6 | 82.2 | CATTTCCAAGAAGCAA[C/T]GCAAAGTAAGTTTTCC | C | 1.555 | macro- |
| | | | | | | albuminuria |
| rs8012459 | 14 | 93.7 | AGGAGAGGGTTAGAAA[A/G]TATGGAATGAAAATAG | G | 2.425 | macro- |
| | | | | | | albuminuria |
| rs882869 | 1 | 264.9 | GAAAACAAAAAGATTA[C/T]GCAAGAGGTCAAGAAG | C | 1.627 | macro- |
| | | | | | | albuminuria |
| rs930056 | 5 | 136.7 | TGGCATCAAAGGTGGG[A/G]ACCTGTCTTTATTATC | A | 0.5638 | macro- |
| | | | | | | albuminuria |
| rs9519630 | 13 | 89.7 | CTGTGGTCCAGCTCTA[C/G]ACGCCAAAAGGAGAGA | G | 0.6007 | macro- |
| | | | | | | albuminuria |
| rs9686988 | 5 | 136.7 | TAAAGTATCCTGTGGA[C/T]CAAGGCTAAGGCTAAG | T | 0.5777 | macro- |
| | | | | | | albuminuria |
| rs9697134 | 9 | 78.3 | TCCCTGATCCATTGAA[C/T]CTAAAAGTCTGAGACT | C | 2.041 | macro- |
| | | | | | | albuminuria |
| rs12511845 | 4 | 132.0 | AACTTAACAGAACTAG[A/T]AAAGAGATCGAAGAAC | A | 0.6743 | micro- |
| | | | | | | albuminuria |
| rs7334517 | 13 | 40.0490868202634 | TTGTGTAGAGTAACTC[C/T]ATGCGTAAACAGTTTC | C | 0.7014 | micro- |
| | | | | | | albuminuria |
| rs7535769 | 1 | 191.801207495317 | AGAAGAGTACACCATG[C/T]CAGTAGTCATCAAAAA | T | 1.361 | micro- |
| | | | | | | albuminuria |
| rs11752805 | 6 | 166.6 | AAGTCTCTGGGTCACA[C/T]TAGCCTTTACCATCTT | T | 1.323 | micro- |
| | | | | | | albuminuria |
| rs17078168 | 8 | 8.56125174027017 | TGGCTCATACTCACCA[A/G]GCTGTGTCCAGAGTCT | A | 0.5574 | micro- |
| | | | | | | albuminuria |
| rs191606 | 1 | 191.806351667483 | CACACAACAATTTAGA[C/T]GGATCTCAAGGGGATT | C | 1.356 | micro- |
| | | | | | | albuminuria |
| rs10071774 | 5 | 68.6778492818504 | AGGGCCTTATGTGTTA[C/T]GTTAAGCACCAGGAAC | T | 0.6582 | micro- |
| | | | | | | albuminuria |
| rs10153433 | 18 | 72.71 | ACTGAATTTATGCTGA[C/T]GTGACTATAATCTTCA | T | 0.8083 | micro- |
| | | | | | | albuminuria |
| rs10471462 | 5 | 68.6773216394885 | ATCCCTGCAAGACCAT[C/T]AATGTAATGAACAGAA | C | 0.6582 | micro- |
| | | | | | | albuminuria |
| rs11183154 | 12 | 59.3291099691675 | CATGTCCAAATGACTA[C/T]CTACAGTTCAAACTGC | T | 0.603 | micro- |
| | | | | | | albuminuria |
| rs11951359 | 5 | 68.6838756257417 | CTGTAAACTTCTATAC[G/T]ACTTTGTACCACCCAT | G | 0.6582 | micro- |
| | | | | | | albuminuria |
| rs1519729. | 11 | 27.1 | TACATCAGGTAATCTA[C/T]ACTGAGCTAATGTAGC | C | 1.216 | micro- |
| | | | | | | albuminuria |
| rs28490018 | 14 | 114.69 | TCATCTGTGAATGAAG[A/G]ATGTCAATATTCAGGA | A | 0.651 | micro- |
| | | | | | | albuminuria |
| rs4702266 | 5 | 12.59 | TAGTGAGGCTCACAGC[A/G]AGTTAGGTAGATGGGA | G | 0.7178 | micro- |
| | | | | | | albuminuria |
| rs6814800 | 4 | 57.19 | TAAGGACACAGATTCA[C/T]GGGTGAGCTTTAGGGG | C | 1.461 | low |
| | | | | | | glomerular |
| | | | | | | filtration |
| rs7195806 | 16 | 124.67689138522 | AAATGGCCCAGGCTGC[A/C]AAAAAGGGGAGCAGCA | C | 0.7345 | micro- |
| | | | | | | albuminuria |

TABLE D

| | chro- | | | Minor | | |
|---|---|---|---|---|---|---|
| | mo- | | | Al- | | |
| rs_ID | some | marshfield | Flanking Sequence | lele | OR | phenotype |
| rs10072570 | 5 | 156.795548639937 | AGTAAAGAGTGGTTGA[C/T]AGTGGAGCTCAGAAGG | T | 0.7572 | complications |
| rs1014534 | 6 | 33.5705389542036 | CTCCATCACTGGGTGC[A/G]ACACTCCTTACAGAGC | G | 1.297 | complications |
| rs1015575 | 6 | 45.7324561804328 | AGTTTTATTACTTACA[A/G]ATCAGTAGCAAGGGAC | A | 1.315 | complications |
| rs10259910 | 7 | 124.188540088814 | TAAGCTGCAATTCTTA[G/T]GTGCTTTAGAGTGTAA | G | 1.275 | complications |
| rs10490076 | 2 | 152.932305300955 | AAGGGTGTGTAATAGA[A/C]TCCTACTCCAACAGGA | C | 0.7275 | complications |
| rs10501441 | 11 | 82.99606230566 | CAGCCCAGAGGAAGAC[A/G]CTGAAGTCCAAAGAGG | A | 0.8532 | complications |
| rs10748424 | 12 | 59.0603764005565 | ACTTGGTTGTCAGGTA[A/G]GTAAGCAAGAAATTCT | A | 1.287 | complications |
| rs10813889 | 9 | 56.7338511554219 | TTAGAGGTATGACATA[A/G]ATGTTCTCTTTCACTC | G | 0.6947 | complications |
| rs10828424 | 10 | 44.6204771222666 | TAATCCGATTTCATCA[A/G]CTCACACTAATCACTA | G | 1.212 | complications |
| rs11846532 | 14 | 109.33344574519 | GGACTTTTATCAGGAA[A/G]CAATAAATGCATTTCA | A | 0.7868 | complications |
| rs11949052 | 5 | 71.7316166440307 | ATTAGCTACAAGTGGA[C/T]AGTAAAGCAGGGAACT | T | 0.6986 | complications |
| rs1243446 | 14 | 8.73231619824919 | AGTGAAACACACATAA[A/G]TGAGCCATGCAGAGAG | G | 0.7676 | complications |
| rs13231053 | 7 | 81.1210501856444 | TCAAAAGACGTGCAAA[A/T]CACCCCAAAATCGTTG | T | 0.8406 | complications |
| rs1464412 | 3 | 61.3891136981571 | AATAAGTGTTCTTTAC[A/G]CACCAATGGCTTCCTA | A | 0.7779 | complications |
| rs16900399 | 5 | 40.9512000425324 | ATGGAAGATGGTTAAA[C/T]GGGGAAAATTGCATTC | T | 0.6947 | complications |
| rs16937197 | 10 | 64.0050134595734 | TACTTCTGAAGCATGA[C/T]GCTAGAGTGGAAAGAA | T | 0.6771 | complications |
| rs16991714 | 22 | 48.9101715732011 | ACTTTAGTTTTTGCCA[A/G]CTGCATATGGAATGGA | G | 0.5361 | complications |
| rs17023520 | 3 | 12.1104454496626 | CATGAGAATTCAATAA[A/T]CTGAGTTATGCCAGTG | T | 0.8873 | complications |
| rs17064029 | 3 | 80.6129131347219 | GTGCTTAAACATATAC[A/G]ACGCATTAAATGAAGC | G | 1.2 | complications |
| rs17117193 | 15 | 10.1129523824034 | GCATAGATAGATGGTA[A/G]CTATGGAAACAATATG | A | 0.9297 | complications |
| rs17171395 | 7 | 58.981812861339 | AATGTTGCCAATGCAA[C/T]CAAGAAAGCCTAGAGT | T | 1.844 | complications |
| rs17362438 | 1 | 82.7811188404582 | TTTGTAGTCTTAGAAC[C/T]AGGATGCTTTACATTT | T | 1.449 | complications |
| rs17362459 | 1 | 82.7811580226607 | TGGTGGGTTCCACCTC[A/G]ATTGCCAACTTTTTAG | A | 1.449 | complications |
| rs17491531 | 22 | 48.0454905521709 | CCCACCATTCAGGGTA[A/G]AAGTTTCCACGCCATC | A | 1.558 | complications |
| rs17823223 | 16 | 69.6794333148209 | CTGTTTTCTAGAGCAA[C/T]GACATAGGGCAGTGGA | T | 0.708 | complications |
| rs186544 | 5 | 45.4577826526895 | CAACTCTTTACCAAAA[A/G]AAGTGAACATACCAAA | G | 1.015 | complications |
| rs199031 | 6 | 40.0511762096637 | TTATTCCCTTATTCCA[C/G]TAATGCAAGGCTTAAT | C | 1.36 | complications |
| rs210664 | 8 | 64.3448570289126 | AGCATCCTGAATGACA[C/G]AGATAAGGACCATATT | G | 1.315 | complications |
| rs2227127 | 6 | 45.7126896072821 | AAAGACATGAAAGGAA[A/G]TGTAGTTTGGTTTGAT | G | 1.284 | complications |
| rs2248225 | 21 | 19.6611909095224 | TCGGTATGTGAGCAAA[C/T]GTTAAATGACATAGTG | T | 1.271 | complications |
| rs2248339 | 21 | 19.6617349210361 | ATCCCAATACAGACTC[C/T]GACTAAGGCTTCCCTT | C | 1.266 | complications |
| rs2352906 | 14 | 51.647591747577 | TGTAAGCATCAGGTAA[C/T]TCATTCGAAAGCTAGA | C | 0.7159 | complications |
| rs2897642 | 11 | 97.8183046783799 | GTAAGTAGTTTATGAA[G/T]CCCTTTCTGATGATGA | T | 0.8284 | complications |
| rs321974 | 7 | 91.3625006907635 | TAGCCTCAGGGCACAG[A/G]CTGAACCAAAAAACAG | G | 1.241 | complications |
| rs3781749 | 11 | 27.2841869296751 | GACGGCAGACTACATA[A/C]CCTGAAGGACTTCACC | C | 0.796 | complications |
| rs4658439 | 1 | 277.396574887442 | CAGCTTACACTGGCCA[C/T]GTGGTATCTTGATGAA | T | 0.772 | complications |
| rs4879628 | 9 | 56.7397507381807 | CAGGTACTGAACTCCC[A/G]GCTCCTCTACAGCCTG | A | 0.7144 | complications |
| rs4918029 | 10 | 125.237896256412 | AAATTTGGCCACAGAA[A/G]CTGGCTAGGAAAAAAC | A | 0.7298 | complications |
| rs4995246 | 4 | 80.6979657321692 | GACAAGGGAAAGTTCC[A/C]AGAAGAGTGATACCTC | A | 1.275 | complications |
| rs6431547 | 2 | 253.112551667033 | TCTATTCACTCTAAAA[C/T]GGCAGCAGGGTTGAGT | C | 0.7538 | complications |
| rs6588759 | 10 | 148.328428499409 | AAGGTCAGCACTGTCA[C/T]GTAAGGTATGTGCTGA | C | 1.31 | complications |
| rs6703571 | 1 | 200.423928547739 | TTTTAAGGTCTTTCGC[C/T]GATTGAATCAGACCCA | C | 1.301 | complications |
| rs6931162 | 6 | 170.133238016092 | TAAAGCAGATTAAAGA[A/G]TATTTGGCTTTTGTTT | G | 0.7715 | complications |
| rs7067701 | 10 | 148.330573087854 | CCCCAAAGACAACCTA[C/G]ATGAGTGAAGAGCCAA | G | 1.317 | complications |
| rs7790213 | 7 | 44.2423285900829 | AAGGGACAGAATGGAA[G/T]AGAGAGTATCAGAGTA | G | 0.8639 | complications |
| rs7838821 | 8 | 6.07406426925523 | TACTCTCAAAATGACC[G/T]GGGTGGTGAATCGAAC | G | 2.121 | complications |
| rs7904011 | 10 | 156.21218752484 | CTAGTAAATACAAAAA[G/T]GACCTAATGCTTAACA | G | 0.7487 | complications |
| rs908366 | 10 | 149.216036090974 | GTTGAAGAGTCGAAAG[C/T]AATAAATGGTGGCCCA | T | 1.277 | complications |
| rs9276440 | 6 | 45.7149422745067 | CCCAGATCATATTTCA[A/T]GTCCAGTAACACAGGA | A | 0.7934 | complications |
| rs9296043 | 6 | 45.7309458950427 | TCTGTGAGAATAGAAA[A/G]TGAATCTTTTGGGAAG | G | 1.302 | complications |
| rs9296044 | 6 | 45.730976671236 | TAGGATGCTTTTGCAG[A/T]AATTAACTTGAGTGGA | T | 1.307 | complications |
| rs9461799 | 6 | 45.695985640688 | ACTGAGTCCCAATGGG[C/T]AACTGATTGCCTCTTT | C | 1.277 | complications |
| rs9469240 | 6 | 45.6963399422308 | TCAAATCAACAATCTC[A/G]CTACACACTCTTATCA | A | 1.275 | complications |
| rs10519051 | 15 | 53.2969660830046 | AATAGTCATAGCAGAA[A/G]TAATACTGATAATATT | G | 1.135 | complications |
| rs2205895 | 1 | 187.4900177488 | ATCTCTAACTGGTGAT[A/G]AAGCATCATGAAATTC | A | 1.205 | complications |
| rs2736172 | 6 | 44.9252179049621 | TCCCTGTCTTTGGACA[C/T]GTAAGAATTGGAGGAA | T | 1.28 | complications |
| rs6547260 | 2 | 101.791914350287 | AACAGAAAATTAAGAT[A/C]AGATTGGCTAACAAGA | A | 1.358 | complications |
| rs6803927 | 3 | 24.7103791650082 | AATGTAGGTCCATAAA[A/C]TGTTTGCATTGATCCA | A | 0.7954 | complications |

TABLE E
Global list of SNPs:

SNPs having the RefSNPID Nos. rs12750223, rs4702266, rs951574, rs13316352, rs3883317,
rs331783, rs1320865, rs6956284, rs12657171, rs11183154, rs7501838, rs4341081,
rs10514925, rs1389798, rs6452689, rs331742, rs10880791, rs4531484, rs12814669,
rs1404868, rs7302315, rs1507216, rs1862230, rs10071774, rs10471462, rs11951359,
rs1507214, rs7966105, rs13173656, rs2667406, rs10121700, rs12413650, rs1280680,
rs4693571, rs10968433, rs2960744, rs2683690, rs7421353, rs6582553, rs10968434,
rs1876681, rs1338195, rs2312150, rs7765427, rs7843358, rs10827750, rs2292343,
rs4679840, rs7616299, rs9851591, rs4937159, rs3917768, rs7240443, rs1875205,
rs16825798, rs9331931, rs243839, rs2389316, rs7750426, rs6478237, rs11258652,
rs6906788, rs11848645, rs130250, rs12695902, rs230157, rs7304649, rs16865535,
rs4775276, rs10743478, rs2370413, rs1540369, rs12505447, rs1177257, rs6814800,
rs1958234, rs7301998, rs16948981, rs16948951, rs7136397, rs6089599, rs28490018,
rs980373, rs12458118, rs424301, rs221873, rs826692, rs6955265, rs2577592, rs17763040,
rs7516762, rs826686, rs10741552, rs221878, rs221871, rs1413896, rs2652148, rs9965472,
rs13276054, rs12965571, rs11577590, rs6470173, rs3128625, rs7317235, rs4239307,
rs4296321, rs1154436, rs430208, rs4968008, rs12443955, rs8094588, rs2449818, rs3019442,
rs1603614, rs7067738, rs2017914, rs9871763, rs714875, rs6981660, rs17191463, rs7839694,
rs1061577, rs3025657, rs3824613, rs4766526, rs1550617, rs6933331, rs7979656, rs6413416,
rs4608810, rs7093687, rs5026429, rs6593482, rs7913948, rs2252884, rs12539689, rs609856,
rs2363683, rs11151820, rs7236071, rs7679643, rs1726673, rs4641492, rs17653276,
rs7919239, rs4734805, rs17266631, rs6761003, rs540391, rs2031817, rs10131139,
rs9557510, rs2590577, rs1653157, rs10487028, rs6903130, rs6465147, rs8081285,
rs7607204, rs10235865, rs10487029, rs8088357, rs13166738, rs12454952, rs1602,
rs6491586, rs751082, rs912369, rs1368761, rs1790866, rs11237675, rs29336, rs1498600,
rs8047671, rs11752725, rs17347351, rs6963627, rs16941252, rs2282775, rs886750,
rs13253981, rs7525955, rs9320994, rs9900205, rs1029969, rs1526591, rs6093764,
rs10189134, rs6429453, rs41530345, rs928579, rs1413618, rs6666581, rs1334237,
rs16830489, rs12127748, rs4321205, rs10748721, rs3750595, rs10786405, rs4280249,
rs4369676, rs4285402, rs2743979, rs6673397, rs10922207, rs17770541, rs7973993,
rs2057028, rs10922204, rs2066926, rs2851870, rs2444242, rs12446492, rs10816424,
rs10978606, rs645170, rs4235846, rs1965193, rs9426484, rs4923872, rs7556846, rs7609541,
rs7570424, rs6750269, rs745165, rs6715897, rs10816423, rs10122952, rs10978601,
rs10978612, rs2292927, rs7972260, rs1549840, rs1292081, rs16901597, rs16901596,
rs6701181, rs2009111, rs2621459, rs902891, rs17692715, rs603151, rs10503257, rs7217945,
rs10804520, rs17154065, rs7194394, rs1258241, rs1258246, rs2413429, rs963264,
rs1258323, rs731017, rs2147102, rs7695033, rs4535211, rs965574, rs12895324, rs6809087,
rs17272796, rs355460, rs9343494, rs1379666, rs1161263, rs355483, rs2873929, rs7655850,
rs2088707, rs4597881, rs4334629, rs3181, rs3909788, rs7690478, rs6810410, rs7549770,
rs7935913, rs1161262, rs4497678, rs10155009, rs4258086, rs11927384, rs4269885,
rs6446391, rs1332457, rs304215, rs7029315, rs7047415, rs16910240, rs6673199, rs6478513,
rs184255, rs4331429, rs2350786, rs17705657, rs7807871, rs1411441, rs1411442, rs882869,
rs1240385, rs10492295, rs10780235, rs470455, rs10867190, rs2220521, rs10867195,
rs12403846, rs12000827, rs9697134, rs3950018, rs7047148, rs7747972, rs2114913,
rs9686988, rs7761326, rs10858809, rs1455857, rs12119983, rs17031374, rs10935316,
rs930056, rs10863962, rs4773709, rs1966908, rs1489922, rs7123971, rs1966909,
rs17095305, rs16931308, rs4790200, rs7947110, rs8012459, rs2026692, rs4883520,
rs9519630, rs4290350, rs1002169, rs1483737, rs17721390, rs7464175, rs17186040,
rs2654981, rs7203172, rs2171304, rs6585304, rs7967892, rs12973797, rs3809485,
rs6589065, rs765, rs7535769, rs11688740, rs13333226, rs980907, rs17600042, rs9540649,
rs634585, rs1327210, rs17680407, rs17680905, rs1913695, rs191606, rs617595, rs7195806,
rs4940275, rs16880395, rs7748736, rs9293123, rs9502593, rs17078168, rs9287229,
rs7243021, rs2936535, rs2324872, rs1218661, rs12511845, rs11752805, rs11707973,
rs2740485, rs4936167, rs4572098, rs2962394, rs7334517, rs4378283, rs1364406,
rs10153433, rs10248275, rs1519729, rs1015575, rs16937197, rs9296044, rs9296043,
rs10813889, rs17491531, rs1243446, rs4658439, rs1464412, rs6431547, rs17823223,
rs2227127, rs10072570, rs16991714, rs4918029, rs6703571, rs10490076, rs16900399,
rs7067701, rs9461799, rs6588759, rs9469240, rs10748424, rs2736172, rs11846532,
rs2248225, rs7838821, rs210664, rs4879628, rs11949052, rs4995246, rs2352906, rs2248339,
rs17362459, rs17362438, rs9276440, rs10259910, rs908366, rs6803927, rs199031,
rs1014534, rs17171395, rs6547260, rs2897642, rs10828424, rs7904011, rs2205895,
rs3781749, rs13231053, rs7790213, rs6931162, rs321974, rs10501441, rs17023520,
rs17064029, rs10519051, rs17117193, and/or rs186544.

TABLE F
List of SNPs associated with T2D risk genes:

rs11810706, rs11810706, rs11810706, rs11810706, rs17562778, rs2481628, rs2481628,
rs2481628, rs2502845, rs2502845, rs2590681, rs704829, rs704829, rs791743, rs791743,
rs16830446, rs735447, rs7579000, rs1061860, rs1061860, rs12485415, rs12485415,
rs12485415, rs1390435, rs1526591, rs1526591, rs1526591, rs1526591, rs1526591,
rs1526591, rs1526591, rs1526591, rs16855137, rs17005998, rs17005998, rs17006048,
rs17006048, rs17006048, rs17023520, rs17050187, rs17606712, rs17606712, rs1920321,
rs6780822, rs6780822, rs7640905, rs7640905, rs7640905, rs7652049, rs7652049, rs7652049,
rs951678, rs9850420, rs9850420, rs9850420, rs9850420, rs9850420, rs10017958,
rs10025265, rs1026975, rs1026975, rs12645522, rs13111631, rs13353636, rs13353636,
rs13435843, rs1385662, rs1385662, rs1450900, rs17031476, rs17031476, rs2359630,
rs2585590, rs29336, rs29336, rs29336, rs4330350, rs4536931, rs7660550, rs7690962,
rs7692314, rs7699961, rs7699961, rs11242144, rs1363697, rs1363697, rs320615, rs398934,
rs4869544, rs4869544, rs4869544, rs4869544, rs4869544, rs4869544, rs4869544, rs4869544,
rs4869544, rs4869544, rs4869544, rs4869544, rs4958117, rs1012424, rs1014534, rs1924664,
rs1924664, rs2260393, rs2261485, rs236369, rs236369, rs236369, rs2452463, rs2814689,
rs3019442, rs3846820, rs3846820, rs3914604, rs4710333, rs586124, rs6455807, rs6926642,
rs6930668, rs6931162, rs7746960, rs9346035, rs9363771, rs9477555, rs10952491,
rs10952491, rs1543265, rs17154998, rs17154998, rs17154998, rs17154998, rs221153,
rs6963627, rs6963627, rs6963627, rs886750, rs4736185, rs7828061, rs9325744, rs9325744,
rs9325744, rs9325744, rs10979779, rs11142029, rs11999319, rs1340186, rs3793561,
rs4310272, rs4310272, rs9886729, rs10998910, rs11193235, rs11195125, rs11195125,
rs11195125, rs11258843, rs11258843, rs11258843, rs11259112, rs11259112, rs12245743,
rs12245743, rs1254947, rs1254947, rs1932604, rs1932604, rs1932604, rs1932604,
rs2698119, rs4445563, rs4881336, rs4881336, rs7071359, rs7071359, rs7071359, rs7073811,
rs7091802, rs908366, rs10765796, rs1878266, rs1406772, rs7969971, rs7969971, rs888152,
rs10508010, rs10508010, rs1630807, rs4771911, rs9517529, rs11848645, rs11848645,
rs11848645, rs10519051, rs10519051, rs11073505, rs11073505, rs16962997, rs17117193,
rs12452883, rs12452883, rs12452883, rs12603207, rs9900205, rs9902506, rs10439056,
rs8094838, rs13040255, rs1891490, rs2426990, rs4347903, rs4812276, rs6013694,
rs6013694, rs6081676, rs6129067, rs6129067, rs942989, rs2839580, rs130250, rs130250,
rs1489880, rs1489890, rs1489894, rs1844461, rs1873230, rs4821241, rs4821283, rs5749770,
rs5749917, rs5754909, rs5755254, rs5755255, rs585124, rs909565.

TABLE G
List of genes associated with the SNPs of the instant application.

| Gene | Description |
|---|---|
| A2BP1 | ataxin 2-binding protein 1 |
| ABCA1 | ATP-binding cassette, sub-family A (ABC1), member 1 |
| ADAMTSL1 | ADAMTS-like 1 |
| AGTR1 | angiotensin II receptor, type 1 |
| AJAP1 (includes | adherens junctions associated protein 1 |
| EG: 55966) | |
| ALK | anaplastic lymphoma receptor tyrosine kinase |
| ALOX5 | arachidonate 5-lipoxygenase |
| AMPH | amphiphysin |
| ANK2 | ankyrin 2, neuronal |
| ANKRD62 | ankyrin repeat domain 62 |
| ANO6 | anoctamin 6 |
| ANXA13 | annexin A13 |
| APBA1 | amyloid beta (A4) precursor protein-binding, family A, |
| | member 1 |
| APBB2 | amyloid beta (A4) precursor protein-binding, family B, |
| | member 2 |
| ARHGAP24 | Rho GTPase activating protein 24 |
| ARMC4 | armadillo repeat containing 4 |
| ASTN2 (includes | astrotactin 2 |
| EG: 23245) | |
| AUTS2 (includes | autism susceptibility candidate 2 |
| EG: 26053) | |
| BAT2 | HLA-B associated transcript 2 |
| BBOX1 | butyrobetaine (gamma), 2-oxoglutarate dioxygenase (gamma- |
| | butyrobetaine hydroxylase) 1 |
| C10ORF116 | chromosome 10 open reading frame 116 |
| C10ORF28 | chromosome 10 open reading frame 28 |
| C10ORF53 | chromosome 10 open reading frame 53 |
| C12ORF35 | chromosome 12 open reading frame 35 |
| C17ORF57 | chromosome 17 open reading frame 57 |
| C4ORF19 | chromosome 4 open reading frame 19 |
| CACNA1C | calcium channel, voltage-dependent, L type, alpha 1C subunit |
| CACNA2D3 | calcium channel, voltage-dependent, alpha 2/delta subunit 3 |
| CACNB2 | calcium channel, voltage-dependent, beta 2 subunit |
| CADPS | Ca++-dependent secretion activator |
| CCBE1 | collagen and calcium binding EGF domains 1 |
| CENPC1 | centromere protein C 1 |
| CHRM2 | cholinergic receptor, muscarinic 2 |
| CHRM3 | cholinergic receptor, muscarinic 3 |
| CHST11 | carbohydrate (chondroitin 4) sulfotransferase 11 |
| CHST15 | carbohydrate (N-acetylgalactosamine 4-sulfate 6-O) |
| | sulfotransferase 15 |
| CLSTN2 | calsyntenin 2 |
| CLU | clusterin |
| CNTN4 | contactin 4 |
| CNTN5 | contactin 5 |
| CNTNAP2 | contactin associated protein-like 2 |
| COG7 | component of oligomeric golgi complex 7 |
| COL22A1 | collagen, type XXII, alpha 1 |
| CREB5 | cAMP responsive element binding protein 5 |
| CSMD1 | CUB and Sushi multiple domains 1 |
| CTNNA2 | catenin (cadherin-associated protein), alpha 2 |
| CTNNA3 | catenin (cadherin-associated protein), alpha 3 |
| CTTNBP2 | cortactin binding protein 2 |
| DBC1 | deleted in bladder cancer 1 |
| DCC | deleted in colorectal carcinoma |
| DCLK1 | doublecortin-like kinase 1 |
| DDX24 | DEAD (Asp-Glu-Ala-Asp) box polypeptide 24 |
| DNER | delta/notch-like EGF repeat containing |
| DOCK1 | dedicator of cytokinesis 1 |
| EFCAB6 | EF-hand calcium binding domain 6 |
| ELAVL3 | ELAV (embryonic lethal, abnormal vision, Drosophila)-like 3 |
| | (Hu antigen C) |
| EME1 | essential meiotic endonuclease 1 homolog 1 (S. pombe) |
| EML6 (includes | echinoderm microtubule associated protein like 6 |
| EG: 400954) | |
| ENOX1 | ecto-NOX disulfide-thiol exchanger 1 |
| EVC2 | Ellis van Creveld syndrome 2 |
| FAM19A1 | family with sequence similarity 19 (chemokine (C-C motif)- |
| | like), member A1 |
| FARP1 | FERM, RhoGEF (ARHGEF) and pleckstrin domain protein 1 |
| | (chondrocyte-derived) |
| FBRSL1 | fibrosin-like 1 |
| FCAMR | Fc receptor, IgA, IgM, high affinity |
| FCN2 | ficolin (collagen/fibrinogen domain containing lectin) 2 |
| | (hucolin) |
| FHIT | fragile histidine triad gene |
| FLJ41200 | hypothetical protein LOC401492 |
| FMN2 | formin 2 |
| FRMD4A | FERM domain containing 4A |
| FSTL4 | follistatin-like 4 |
| FTO | fat mass and obesity associated |
| GABRB2 | gamma-aminobutyric acid (GABA) A receptor, beta 2 |
| GABRB3 | gamma-aminobutyric acid (GABA) A receptor, beta 3 |
| GABRG1 | gamma-aminobutyric acid (GABA) A receptor, gamma 1 |
| GALNTL4 | UDP-N-acetyl-alpha-D-galactosamine: polypeptide N- |
| | acetylgalactosaminyltransferase-like 4 |
| GAPDHS | glyceraldehyde-3-phosphate dehydrogenase, spermatogenic |
| GGA2 | golgi-associated, gamma adaptin ear containing, ARF binding |
| | protein 2 |
| GPR37L1 | G protein-coupled receptor 37 like 1 |
| GRIA1 | glutamate receptor, ionotropic, AMPA 1 |
| GRIN2A | glutamate receptor, ionotropic, N-methyl D-aspartate 2A |
| HLA-DQA2 | major histocompatibility complex, class II, DQ alpha 2 |
| HS3ST4 | heparan sulfate (glucosamine) 3-O-sulfotransferase 4 |
| IGF1R | insulin-like growth factor 1 receptor |
| JAZF1 | JAZF zinc finger 1 |
| JPH2 | junctophilin 2 |
| KCNQ5 | potassium voltage-gated channel, KQT-like subfamily, |
| | member 5 |
| KIAA1644 | KIAA1644 |
| KIF13A | kinesin family member 13A |
| KIRREL3 | kin of IRRE like 3 (Drosophila) |
| KIT | v-kit Hardy-Zuckerman 4 feline sarcoma viral oncogene |
| | homolog |
| LARGE | like-glycosyltransferase |
| LIMS1 | LIM and senescent cell antigen-like domains 1 |
| LINGO2 | leucine rich repeat and Ig domain containing 2 |
| LOC642340 | hypothetical LOC642340 |
| LOXL2 | lysyl oxidase-like 2 |
| LRRIQ1 | leucine-rich repeats and IQ motif containing 1 |
| LSAMP | limbic system-associated membrane protein |
| MAD1L1 | MAD1 mitotic arrest deficient-like 1 (yeast) |
| MAGI2 | membrane associated guanylate kinase, WW and PDZ domain |
| | containing 2 |
| MAML2 | mastermind-like 2 (Drosophila) |
| MFSD1 | major facilitator superfamily domain containing 1 |
| MGAM | maltase-glucoamylase (alpha-glucosidase) |
| MGAT4C | mannosyl (alpha-1,3-)-glycoprotein beta-1,4-N- |
| | acetylglucosaminyltransferase, isozyme C (putative) |
| MMP2 | matrix metallopeptidase 2 (gelatinase A, 72 kDa gelatinase, |
| | 72 kDa type IV collagenase) |
| MOBKL2B | MOB1, Mps One Binder kinase activator-like 2B (yeast) |
| MRPL27 | mitochondrial ribosomal protein L27 |
| MYO18B | myosin XVIIIB |
| MYRIP | myosin VIIA and Rab interacting protein |
| MYT1 | myelin transcription factor 1 |
| NAALADL2 | N-acetylated alpha-linked acidic dipeptidase-like 2 |
| NDRG2 | NDRG family member 2 |
| NELL1 | NEL-like 1 (chicken) |
| NKAIN2 | Na+/K+ transporting ATPase interacting 2 |
| NOS1AP | nitric oxide synthase 1 (neuronal) adaptor protein |
| NPAS3 | neuronal PAS domain protein 3 |
| NRG1 | neuregulin 1 |
| NTRK3 | neurotrophic tyrosine kinase, receptor, type 3 |
| OAF | OAF homolog (Drosophila) |
| ODZ4 (includes | odz, odd Oz/ten-m homolog 4 (Drosophila) |
| EG: 26011) | |
| OPCML | opioid binding protein/cell adhesion molecule-like |
| PARD3B | par-3 partitioning defective 3 homolog B (C. elegans) |
| PARK2 | Parkinson disease (autosomal recessive, juvenile) 2, parkin |
| PDE11A | phosphodiesterase 11A |
| PDE3A | phosphodiesterase 3A, cGMP-inhibited |
| PDE4D | phosphodiesterase 4D, cAMP-specific |
| PDE7B | phosphodiesterase 7B |
| PDILT | protein disulfide isomerase-like, testis expressed |
| PDZD2 | PDZ domain containing 2 |
| PHF17 | PHD finger protein 17 |
| PKD1L2 | polycystic kidney disease 1-like 2 |
| PKNOX2 | PBX/knotted 1 homeobox 2 |
| PLCG2 | phospholipase C, gamma 2 (phosphatidylinositol-specific) |
| PLCL2 | phospholipase C-like 2 |
| PRKCA | protein kinase C, alpha |
| PTPRD | protein tyrosine phosphatase, receptor type, D |
| PTPRT | protein tyrosine phosphatase, receptor type, T |
| RAB17 | RAB17, member RAS oncogene family |
| RNF180 | ring finger protein 180 |
| RORA | RAR-related orphan receptor A |
| SCHIP1 | schwannomin interacting protein 1 |
| SDK1 | sidekick homolog 1, cell adhesion molecule (chicken) |
| SEC14L3 | SEC14-like 3 (S. cerevisiae) |
| SELP | selectin P (granule membrane protein 140 kDa, antigen CD62) |
| SEMA6D | sema domain, transmembrane domain (TM), and cytoplasmic |
| | domain, (semaphorin) 6D |
| SENP5 (includes | SUMO1/sentrin specific peptidase 5 |
| EG: 205564) | |
| SGIP1 | SH3-domain GRB2-like (endophilin) interacting protein 1 |
| SIK3 | SIK family kinase 3 |
| SLC6A2 | solute carrier family 6 (neurotransmitter transporter, |
| | noradrenalin), member 2 |
| SOX5 (includes | SRY (sex determining region Y)-box 5 |
| EG: 6660) | |
| SPNS2 (includes | spinster homolog 2 (Drosophila) |
| EG: 124976) | |
| SSR1 | signal sequence receptor, alpha |
| SULF1 | sulfatase 1 |
| SYNE1 | spectrin repeat containing, nuclear envelope 1 |
| SYT9 | synaptotagmin IX |
| TAF4 | TAF4 RNA polymerase II, TATA box binding protein (TBP)- |
| | associated factor, 135 kDa |
| TMEM132D | transmembrane protein 132D |
| TNFRSF1B | tumor necrosis factor receptor superfamily, member 1B |
| TOX | thymocyte selection-associated high mobility group box |
| TRPV1 | transient receptor potential cation channel, subfamily V, |
| | member 1 |
| TRUB1 | TruB pseudouridine (psi) synthase homolog 1 (E. coli) |
| TSPAN19 | tetraspanin 19 |
| UMOD | uromodulin |
| UNC5C | unc-5 homolog C (C. elegans) |
| WDR59 | WD repeat domain 59 |
| WWOX | WW domain containing oxidoreductase |
| ZNF181 | zinc finger protein 181 |

1. A method for predicting increased risk of developing a complication which is myocardial infarction, stroke, or kidney complication in a subject affected by T2D, comprising detecting, in a sample obtained from said subject, at least one genetic feature which is
(a) a SNP having the RefSNPID Nos. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544;
(b) at least one SNP which is in linkage disequilibrium with at least one SNP of (a).

2. The method according to claim 1, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264, at least 265, at least 266, at least 267, at least 268, at least 269, at least 270, at least 271, at least 272, at least 273, at least 274, at least 275, at least 276, at least 277, at least 278, at least 279, at least 280, at least 281, at least 282, at least 283, at least 284, at least 285, at least 286, at least 287, at least 288, at least 289, at least 290, at least 291, at least 292, at least 293, at least 294, at least 295, at least 296, at least 297, at least 298, at least 299, at least 300, at least 301, at least 302, at least 303, at least 304, at least 305, at least 306, at least 307, at least 308, at least 309, at least 310, at least 311, at least 312, at least 313, at least 314, at least 315, at least 316, at least 317, at least 318, at least 319, at least 320, at least 321, at least 322, at least 323, at least 324, at least 325, at least 326, at least 327, at least 328, at least 329, at least 330, at least 331, at least 332, at least 333, at least 334, at least 335, at least 336, at least 337, at least 338, at least 339, at least 340, at least 341, at least 342, at least 343, at least 344, at least 345, at least 346, at least 347, at least 348, at least 349, at least 350, at least 351, at least 352, at least 353, at least 354, at least 355, at least 356, at least 357, at least 358, at least 359, at least 360, at least 361, at least 362, at least 363, at least 364, at least 365, at least 366, at least 367, at least 368, at least 369, at least 370, at least 371, at least 372, at least 373, at least 374, at least 375, at least 376, at least 377, at least 378, at least 379, at least 380, at least 381, at least 382, at least 383, at least 384, at least 385, at least 386, at least 387, at least 388, at least 389, at least 390, at least 391, at least 392, at least 393, at least 394, at least 395, at least 396, at least 397, at least 398, at least 399, at least 400, at least 401, at least 402, at least 403, at least 404, at least 405, at least 406, at least 407, at least 408, at least 409, at least 410, at least 411, at least 412, at least 413, at least 414, at least 415, at least 416, at least 417, at least 418, at least 419, at least 420, at least 421, at least 422, at least 423, at least 424, at least 425, at least 426, at least 427, at least 428, at least 429, at least 430, at least 431, at least 432, at least 433, at least 434, at least 435, at least 436, at least 437, at least 438, at least 439, at least 440, at least 441, at least 442, at least 443, at least 444, at least 445, or more of said SNPs.

3. The method according to claim 1, wherein said complication is stroke and said method comprises detection of at least one SNP having the RefSNP ID No. rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, and/or rs10189134.

4. The method according to claim 3, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, or more of the aforementioned SNPs.

5. The method according to claim 1, wherein said complication is MI and said method comprises detection of at least one SNP having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, and/or rs17191463.

6. The method according to claim 5, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, or more of the SNPs.

7. The method according to claim 1, wherein said complication is kidney-related complication and said method comprises detection of at least one SNP having the RefSNP ID No. rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs 10922204, rs2066926, rs3917768, rs2851870, rs2444242, rs 12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs7765427, rs963264, rs1258323, rs28490018, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs6814800, rs3909788, rs7690478, rs6810410, rs7549770, rs7935913, rs1161262, rs4497678, rs10155009, rs4258086, rs11927384, rs4269885, rs16825798, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs11848645, rs7203172, rs2171304, rs6585304, rs7967892, rs7501838, rs3883317, rs12973797, rs3809485, rs6589065, rs765, rs7535769, rs11688740, rs10514925, rs13333226, rs4702266, rs980907, rs17600042, rs9540649, rs634585, rs1327210, rs17680407, rs17680905, rs1913695, rs191606, rs11951359, rs10071774, rs10471462, rs617595, rs7195806, rs4940275, rs16880395, rs7748736, rs9293123, rs9502593, rs17078168, rs9287229, rs7243021, rs2936535, rs11183154, rs2324872, rs1218661, rs12511845, rs11752805, rs11707973, rs2740485, rs4936167, rs4572098, rs243839, rs2962394, rs7334517, rs130250, rs4378283, rs1364406, rs10153433, rs10248275, and/or rs1519729.

8. The method according to claim 7, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, or more of the aforementioned SNPs.

9. The method according to claim 1, comprising detecting a SNP having the RefSNP ID No. rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544.

10. The method according to claim 9, comprising detecting a combination of SNPs, wherein said combination comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, or more of the aforementioned SNPs.

11. The method according to claim 1, comprising detecting
(b) at least one SNP which is in linkage disequilibrium with at least one SNP of (a) at an r² threshold of at least 0.8.

12. The method according to claim 1, wherein an allele associated with said at least one SNP is a minor allele.

13. The method according to claim 1, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.

14. The method according to claim 3, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.

15. The method according to claim 5, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.

16. The method according to claim 7, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.

17. The method according to claim 9, with the proviso that said at least one SNP is not a SNP having the RefSNPID No. rs11810706, rs11810706, rs11810706, rs11810706, rs17562778, rs2481628, rs2481628, rs2481628, rs2502845, rs2502845, rs2590681, rs704829, rs704829, rs791743, rs791743, rs16830446, rs735447, rs7579000, rs1061860, rs1061860, rs12485415, rs12485415, rs12485415, rs1390435, rs1526591, rs1526591, rs1526591, rs1526591, rs1526591, rs1526591, rs1526591, rs1526591, rs16855137, rs17005998, rs17005998, rs17006048, rs17006048, rs17006048, rs17023520, rs17050187, rs17606712, rs17606712, rs1920321, rs6780822, rs6780822, rs7640905, rs7640905, rs7640905, rs7652049, rs7652049, rs7652049, rs951678, rs9850420, rs9850420, rs9850420, rs9850420, rs9850420, rs10017958, rs10025265, rs1026975, rs1026975, rs12645522, rs13111631, rs13353636, rs13353636, rs13435843, rs1385662, rs1385662, rs1450900, rs17031476, rs17031476, rs2359630, rs2585590, rs29336, rs29336, rs29336, rs4330350, rs4536931, rs7660550, rs7690962, rs7692314, rs7699961, rs7699961, rs11242144, rs1363697, rs1363697, rs320615, rs398934, rs4869544, rs4869544, rs4869544, rs4869544, rs4869544, rs4869544, rs4869544, rs4869544, rs4869544, rs4869544, rs4869544, rs4869544, rs4958117, rs1012424, rs1014534, rs1924664, rs1924664, rs2260393, rs2261485, rs236369, rs236369, rs236369, rs2452463, rs2814689, rs3019442, rs3846820, rs3846820, rs3914604, rs4710333, rs586124, rs6455807, rs6926642, rs6930668, rs6931162, rs7746960, rs9346035, rs9363771, rs9477555, rs10952491, rs10952491, rs1543265, rs17154998, rs17154998, rs17154998, rs17154998, rs221153, rs6963627, rs6963627, rs6963627, rs886750, rs4736185, rs7828061, rs9325744, rs9325744, rs9325744, rs9325744, rs10979779, rs11142029, rs11999319, rs1340186, rs3793561, rs4310272, rs4310272, rs9886729, rs10998910, rs11193235, rs11195125, rs11195125, rs11195125, rs11258843, rs11258843, rs11258843, rs11259112, rs11259112, rs12245743, rs12245743, rs1254947, rs1254947, rs1932604, rs1932604, rs1932604, rs1932604, rs2698119, rs4445563, rs4881336, rs4881336, rs7071359, rs7071359, rs7071359, rs7073811, rs7091802, rs908366, rs10765796, rs1878266, rs1406772, rs7969971, rs7969971, rs888152, rs10508010, rs10508010, rs1630807, rs4771911, rs9517529, rs11848645, rs11848645, rs11848645, rs10519051, rs10519051, rs11073505, rs11073505, rs16962997, rs17117193, rs12452883, rs12452883, rs12452883, rs12603207, rs9900205, rs9902506, rs10439056, rs8094838, rs13040255, rs1891490, rs2426990, rs4347903, rs4812276, rs6013694, rs6013694, rs6081676, rs6129067, rs6129067, rs942989, rs2839580, rs130250, rs130250, rs1489880, rs1489890, rs1489894, rs1844461, rs1873230, rs4821241, rs4821283, rs5749770, rs5749917, rs5754909, rs5755254, rs5755255, rs585124, and rs909565.

18. A kit for predicting a complication which is myocardial infarction, stroke or kidney complication in a subject affected by type-2 diabetes (T2D) comprising in one or more packages
(a) at least one oligonucleotide that specifically hybridizes to at least one allele of a SNP having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) an oligonucleotide which is the complement of (a);
and one or more reagents for the detection of said oligonucleotide.

19. The kit according to claim 18, which comprises a combination of oligonucleotides which hybridize to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264, at least 265, at least 266, at least 267, at least 268, at least 269, at least 270, at least 271, at least 272, at least 273, at least 274, at least 275, at least 276, at least 277, at least 278, at least 279, at least 280, at least 281, at least 282, at least 283, at least 284, at least 285, at least 286, at least 287, at least 288, at least 289, at least 290, at least 291, at least 292, at least 293, at least 294, at least 295, at least 296, at least 297, at least 298, at least 299, at least 300, at least 301, at least 302, at least 303, at least 304, at least 305, at least 306, at least 307, at least 308, at least 309, at least 310, at least 311, at least 312, at least 313, at least 314, at least 315, at least 316, at least 317, at least 318, at least 319, at least 320, at least 321, at least 322, at least 323, at least 324, at least 325, at least 326, at least 327, at least 328, at least 329, at least 330, at least 331, at least 332, at least 333, at least 334, at least 335, at least 336, at least 337, at least 338, at least 339, at least 340, at least 341, at least 342, at least 343, at least 344, at least 345, at least 346, at least 347, at least 348, at least 349, at least 350, at least 351, at least 352, at least 353, at least 354, at least 355, at least 356, at least 357, at least 358, at least 359, at least 360, at least 361, at least 362, at least 363, at least 364, at least 365, at least 366, at least 367, at least 368, at least 369, at least 370, at least 371, at least 372, at least 373, at least 374, at least 375, at least 376, at least 377, at least 378, at least 379, at least 380, at least 381, at least 382, at least 383, at least 384, at least 385, at least 386, at least 387, at least 388, at least 389, at least 390, at least 391, at least 392, at least 393, at least 394, at least 395, at least 396, at least 397, at least 398, at least 399, at least 400, at least 401, at least 402, at least 403, at least 404, at least 405, at least 406, at least 407, at least 408, at least 409, at least 410, at least 411, at least 412, at least 413, at least 414, at least 415, at least 416, at least 417, at least 418, at least 419, at least 420, at least 421, at least 422, at least 423, at least 424, at least 425, at least 426, at least 427, at least 428, at least 429, at least 430, at least 431, at least 432, at least 433, at least 434, at least 435, at least 436, at least 437, at least 438, at least 439, at least 440, at least 441, at least 442, at least 443, at least 444, at least 445, or at least 446 of the SNPs of claim 18.

20. A method for preventing, treating or reducing the risk of T2D or a T2D related complication which is myocardial infarction, stroke, albuminuria or declining kidney complication in a subject in need thereof, comprising administrating to said subject a therapeutic agent which
(a) alters the activity or levels of a polypeptide encoded by at least one risk gene listed in Table G or one gene associated with a SNP having the RefSNP ID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544; or
(b) alters the expression of at least one polymorphism disclosed in (a); or
(c) alters the specific metabolic or other biologically related pathway implicating the risk gene of (a).

21. A method for identifying a subject for preventive therapeutic action against type 2 diabetes (T2D) related complication, comprising detecting, in a sample obtained from said subject, at least one genetic feature which is
(a) at least one single nucleotide polymorphism (SNP) having the RefSNPID No. rs12750223, rs4702266, rs951574, rs13316352, rs3883317, rs331783, rs1320865, rs6956284, rs12657171, rs11183154, rs7501838, rs4341081, rs10514925, rs1389798, rs6452689, rs331742, rs10880791, rs4531484, rs12814669, rs1404868, rs7302315, rs1507216, rs1862230, rs10071774, rs10471462, rs11951359, rs1507214, rs7966105, rs13173656, rs2667406, rs10121700, rs12413650, rs1280680, rs4693571, rs10968433, rs2960744, rs2683690, rs7421353, rs6582553, rs10968434, rs1876681, rs1338195, rs2312150, rs7765427, rs7843358, rs10827750, rs2292343, rs4679840, rs7616299, rs9851591, rs4937159, rs3917768, rs7240443, rs1875205, rs16825798, rs9331931, rs243839, rs2389316, rs7750426, rs6478237, rs11258652, rs6906788, rs11848645, rs130250, rs12695902, rs230157, rs7304649, rs16865535, rs4775276, rs10743478, rs2370413, rs1540369, rs12505447, rs1177257, rs6814800, rs1958234, rs7301998, rs16948981, rs16948951, rs7136397, rs6089599, rs28490018, rs980373, rs12458118, rs424301, rs221873, rs826692, rs6955265, rs2577592, rs17763040, rs7516762, rs826686, rs10741552, rs221878, rs221871, rs1413896, rs2652148, rs9965472, rs13276054, rs12965571, rs11577590, rs6470173, rs3128625, rs7317235, rs4239307, rs4296321, rs1154436, rs430208, rs4968008, rs12443955, rs8094588, rs2449818, rs3019442, rs1603614, rs7067738, rs2017914, rs9871763, rs714875, rs6981660, rs17191463, rs7839694, rs1061577, rs3025657, rs3824613, rs4766526, rs1550617, rs6933331, rs7979656, rs6413416, rs4608810, rs7093687, rs5026429, rs6593482, rs7913948, rs2252884, rs12539689, rs609856, rs2363683, rs11151820, rs7236071, rs7679643, rs1726673, rs4641492, rs17653276, rs7919239, rs4734805, rs17266631, rs6761003, rs540391, rs2031817, rs10131139, rs9557510, rs2590577, rs1653157, rs10487028, rs6903130, rs6465147, rs8081285, rs7607204, rs10235865, rs10487029, rs8088357, rs13166738, rs12454952, rs1602, rs6491586, rs751082, rs912369, rs1368761, rs1790866, rs11237675, rs29336, rs1498600, rs8047671, rs11752725, rs17347351, rs6963627, rs16941252, rs2282775, rs886750, rs13253981, rs7525955, rs9320994, rs9900205, rs1029969, rs1526591, rs6093764, rs10189134, rs6429453, rs41530345, rs928579, rs1413618, rs6666581, rs1334237, rs16830489, rs12127748, rs4321205, rs10748721, rs3750595, rs10786405, rs4280249, rs4369676, rs4285402, rs2743979, rs6673397, rs10922207, rs17770541, rs7973993, rs2057028, rs10922204, rs2066926, rs2851870, rs2444242, rs12446492, rs10816424, rs10978606, rs645170, rs4235846, rs1965193, rs9426484, rs4923872, rs7556846, rs7609541, rs7570424, rs6750269, rs745165, rs6715897, rs10816423, rs10122952, rs10978601, rs10978612, rs2292927, rs7972260, rs1549840, rs1292081, rs16901597, rs16901596, rs6701181, rs2009111, rs2621459, rs902891, rs17692715, rs603151, rs10503257, rs7217945, rs10804520, rs17154065, rs7194394, rs1258241, rs1258246, rs2413429, rs963264, rs1258323, rs731017, rs2147102, rs7695033, rs4535211, rs965574, rs12895324, rs6809087, rs17272796, rs355460, rs9343494, rs1379666, rs1161263, rs355483, rs2873929, rs7655850, rs2088707, rs4597881, rs4334629, rs3181, rs3909788, rs7690478, rs6810410, rs7549770, rs7935913, rs1161262, rs4497678, rs10155009, rs4258086, rs11927384, rs4269885, rs6446391, rs1332457, rs304215, rs7029315, rs7047415, rs16910240, rs6673199, rs6478513, rs184255, rs4331429, rs2350786, rs17705657, rs7807871, rs1411441, rs1411442, rs882869, rs1240385, rs10492295, rs10780235, rs470455, rs10867190, rs2220521, rs10867195, rs12403846, rs12000827, rs9697134, rs3950018, rs7047148, rs7747972, rs2114913, rs9686988, rs7761326, rs10858809, rs1455857, rs12119983, rs17031374, rs10935316, rs930056, rs10863962, rs4773709, rs1966908, rs1489922, rs7123971, rs1966909, rs17095305, rs16931308, rs4790200, rs7947110, rs8012459, rs2026692, rs4883520, rs9519630, rs4290350, rs1002169, rs1483737, rs17721390, rs7464175, rs17186040, rs2654981, rs7203172, rs2171304, rs6585304, rs7967892, rs12973797, rs3809485, rs6589065, rs765, rs7535769, rs11688740, rs13333226, rs980907, rs17600042, rs9540649, rs634585, rs1327210, rs17680407, rs17680905, rs1913695, rs191606, rs617595, rs7195806, rs4940275, rs16880395, rs7748736, rs9293123, rs9502593, rs17078168, rs9287229, rs7243021, rs2936535, rs2324872, rs1218661, rs12511845, rs11752805, rs11707973, rs2740485, rs4936167, rs4572098, rs2962394, rs7334517, rs4378283, rs1364406, rs10153433, rs10248275, rs1519729, rs1015575, rs16937197, rs9296044, rs9296043, rs10813889, rs17491531, rs1243446, rs4658439, rs1464412, rs6431547, rs17823223, rs2227127, rs10072570, rs16991714, rs4918029, rs6703571, rs10490076, rs16900399, rs7067701, rs9461799, rs6588759, rs9469240, rs10748424, rs2736172, rs11846532, rs2248225, rs7838821, rs210664, rs4879628, rs11949052, rs4995246, rs2352906, rs2248339, rs17362459, rs17362438, rs9276440, rs10259910, rs908366, rs6803927, rs199031, rs1014534, rs17171395, rs6547260, rs2897642, rs10828424, rs7904011, rs2205895, rs3781749, rs13231053, rs7790213, rs6931162, rs321974, rs10501441, rs17023520, rs17064029, rs10519051, rs17117193, and/or rs186544;
(b) at least one SNP which is in linkage disequilibrium with at least one SNP of (a).
wherein the detection of said genetic feature in said subject correlates with the eligibility of said subject for said preventive therapeutic action.

22. The method according to claim 21, comprising detecting
(b) at least one SNP which is in linkage disequilibrium with at least one SNP of (a) at an r² threshold of at least 0.8.

23. The method according to claim 21, wherein an allele associated with said at least one SNP is a minor allele.

24. The method according to claim 21, wherein the complication is myocardial infarction, stroke, or kidney complication.

25. A method for predicting increased risk of developing a complication which is myocardial infarction, stroke, or kidney complication in a subject affected by T2D, comprising detecting, in a sample obtained from said subject, at least one genetic feature which is single nucleotide polymorphism (SNP) or short tandem repeat (STR) of at least one gene which is listed in Table G, wherein the detection of said genetic feature in said subject correlates with said increased risk of developing at least one of said complication.
